SEQUENCE LISTING (54 sequences)

SEQ ID NO: 1 - protein, 103 aa, Vibrio cholerae
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 2 - DNA, 309 bp, Vibrio cholerae
  source 1..309: mol_type = other DNA
acaccacagaacatcacagatctctgcgccgagtaccacaacacccagatccacacgctcaacgacaagatcttcagctacaccgagagcctcgctggcaagcgcgagatggccattatcaccttcaagaacggcgccaccttccaggttgaggtgccaggctctcagcacatcgactcccagaagaaagccatcgagcgcatgaaggacaccctcaggatcgcctacctcaccgaggccaaggttgagaagctctgcgtgtggaacaacaagaccccgcatgccattgccgccatctctatggccaat

SEQ ID NO: 3 - protein, 107 aa, Mus musculus
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRPTGK

SEQ ID NO: 4 - DNA, 321 bp, Mus musculus
  source 1..321: mol_type = other DNA
ggctccgtgagagccccgcaagtgtatgttcttccgccgccagaggaagagatgaccaagaagcaagtcaccctgacctgcatggtgaccgacttcatgccagaggatatctacgtcgaatggaccaacaacggcaagaccgagctgaactacaagaacaccgagccggtgctcgactccgacggctcctacttcatgtactccaagctccgcgtcgagaagaagaactgggtcgagcgcaactcctactcctgctccgttgttcatgagggcctccacaaccaccacaccaccaagtctttctcccggccaactggcaag

SEQ ID NO: 5 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 321 bp, Homo sapiens
  source 1..321: mol_type = other DNA
ggccagccaagagagccacaggtttacacacttccgccgtccagggatgagctgaccaagaaccaggtgtccctgacctgcctcgtgaagggcttctacccatccgatatcgccgtcgagtgggagtccaatggccagcctgagaacaattacaagaccacaccgccggtgctcgactccgatggctcattcttcctgtactccaagctgaccgtcgacaaatccagatggcaacagggcaacgtgttctcctgctccgttatgcacgaggccctccacaaccactacacccagaagtctctcagcctctcgccaggcaag

SEQ ID NO: 7 - protein, 110 aa, Mus musculus
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 8 - DNA, 330 bp, Mus musculus
  source 1..330: mol_type = other DNA
gcgccaaatcttcttggcggcccatccgtgttcatcttcccgccgaagatcaaggacgtgctcatgatctccctctcgccgatcgtgacatgcgtggtggtggatgtgtccgaggacgatccggatgtccagatctcctggttcgtgaacaacgtcgaggtgcacaccgcgcagacacaaacacaccgcgaggactacaatagcaccctcagagtggtgagcgccctgccaatccagcaccaggattggatgtccgggaaagaattcaagtgcaaggtcaacaacaaggacctgccagcgccgatcgagaggaccatctctaagccaaag

SEQ ID NO: 9 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 10 - DNA, 330 bp, Homo sapiens
  source 1..330: mol_type = other DNA
gctccagaacttcttggcggcccatccgtgtttctgttcccgccgaagccaaaggacacgctgatgatctctcgcaccccagaggtgacatgcgtggtggtggatgtgtcccacgaggaccctgaggtgaagttcaactggtatgtggacggcgtcgaggtgcacaacgccaagacaaagccgcgcgaggaacagtacaactccacctacagagtggtgtccgtgctcaccgtgctccaccaggattggctgaacgggaaagaatacaaatgcaaggtgtccaacaaggcgctcccagcgccgatcgaaaagaccatctctaaggcgaag

SEQ ID NO: 11 - protein, 16 aa, Mus musculus
EPRGPTNKPSPPCKCP

SEQ ID NO: 12 - DNA, 48 bp, Mus musculus
  source 1..48: mol_type = other DNA
gagccaaggggcccgacaaacaagccttctccaccatgcaagtgccca

SEQ ID NO: 13 - protein, 15 aa, Homo sapiens
EPKSSDKTHTCPPCP

SEQ ID NO: 14 - DNA, 45 bp, Homo sapiens
  source 1..45: mol_type = other DNA
gagccgaagtcctccgacaagacccatacttgcccaccgtgtcca

SEQ ID NO: 15 - protein, 10 aa, Mus musculus
ASSTKVDKKI

SEQ ID NO: 16 - DNA, 30 bp, Mus musculus
  source 1..30: mol_type = other DNA
gccagctctaccaaggtggacaagaagatc

SEQ ID NO: 17 - protein, 10 aa, Homo sapiens
ASNTKVDKKV

SEQ ID NO: 18 - DNA, 30 bp, Homo sapiens
  source 1..30: mol_type = other DNA
gcctccaataccaaggtggacaagaaggtc

SEQ ID NO: 19 - protein, 103 aa, Dengue virus
KGMSYAMCTGKFKLEKEVAETQHGTILIKVKYEGDGAPCKIPFEIQDVEKKHVNGRLITANPIVTDKESPVNIEAEPPFGDSYIVIGVGDKALKLNWFKKGSS

SEQ ID NO: 20 - DNA, 309 bp, Dengue virus
  source 1..309: mol_type = other DNA
aagggcatgtcctacgctatgtgcaccggcaagttcaagttggagaaggaggtggctgagacccagcacggcaccatcttgatcaaggtgaagtacgagggcgatggcgctccttgcaagatccctttcgagatccaggatgtggagaagaagcacgtgaacggaaggttgatcaccgctaaccctatcgtgaccgataaggagtcccctgtgaacatcgaggctgagcctcctttcggcgattcctacatcgtgatcggcgtgggcgataaggctttgaagttgaactggttcaagaagggttcttca

SEQ ID NO: 21 - protein, 201 aa, SARS-CoV-2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 22 - DNA, 603 bp, SARS-CoV-2
  source 1..603: mol_type = other DNA
cagccaaccgaatccattgtgcgcttcccgaacatcaccaatctgtgcccattcggcgaggtgttcaacgccacaagattcgcctctgtgtacgcctggaaccgcaagcgcatctccaattgcgtggccgactactccgtgctctacaactctgccagcttctccacgttcaagtgctacggcgtgtccccgaccaagctgaacgatctctgcttcaccaacgtgtacgccgactccttcgtgatcagaggcgacgaggtgaggcaaattgccccaggccagacaggcaagatcgccgactacaactacaagctcccggacgacttcaccggctgcgtgatcgcttggaactccaacaacctcgactccaaggtcggcggcaactacaattacctctaccgcctcttccgcaagtccaacctcaagccgttcgagcgcgatatctccaccgagatctatcaggccggaagcaccccatgcaatggcgtcgaaggcttcaactgctacttcccgctccagtcctacggcttccagcctacaaatggcgtgggctaccaaccgtacagggtcgtcgttctcagcttcgagctgctccatgctcca

SEQ ID NO: 23 - protein, 200 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFAGGGGGMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 24 - DNA, 600 bp, Mycobacterium tuberculosis
  source 1..600: mol_type = other DNA
atgactgagcagcagtggaattttgctggtattgaggctgctgcttctgctattcagggtaatgttacttctattcattctcttcttgatgagggtaaacagtctcttactaagttggctgctgcttggggtggttctggttctgaggcttaccagggtgttcagcagaagtgggatgctactgctactgagcttaacaatgctcttcagaatcttgctaggactatttctgaggctggtcaggctatggcttctactgagggtaatgttactggtatgtttgctggtggtggtggtggtatggctgagatgaagactgatgctgctactcttgctcaggaggctggtaattttgagaggatttctggagatttgaagactcagattgatcaggttgagtctactgctggttctcttcagggtcagtggaggggtgctgctggtactgctgctcaggctgctgttgttaggtttcaggaggctgctaataagcagaagcaggagcttgatgagatttctactaatattaggcaggctggtgttcagtactctagggctgatgaggagcagcagcaggctctttcttctcagatgggtttt

SEQ ID NO: 25 - protein, 19 aa, Homo sapiens
MELGLSWIFLLAILKGVQC

SEQ ID NO: 26 - DNA, 57 bp, Homo sapiens
  source 1..57: mol_type = other DNA
atggaactcggcctttcttggattttcttgctcgctatcctcaagggcgtgcaatgc

SEQ ID NO: 27 - protein, 25 aa, Oryza sativa
MKNTSSLCLLLLVVLCSLTCNSGQA

SEQ ID NO: 28 - DNA, 75 bp, Oryza sativa
  source 1..75: mol_type = other DNA
atgaagaatacctccagcctctgcctcctcctcctggtggttctctgctccctcacatgcaattccggccaggcc

SEQ ID NO: 29 - protein, 6 aa, unidentified
SEKDEL

SEQ ID NO: 30 - DNA, 18 bp, unidentified
  source 1..18: mol_type = other DNA
tccgagaaggatgagctt

SEQ ID NO: 31 - protein, 6 aa, unidentified
GPGPGS

SEQ ID NO: 32 - DNA, 18 bp, unidentified
  source 1..18: mol_type = other DNA
ggcccaggcccgggatcc

SEQ ID NO: 33 - protein, 17 aa, unidentified
AEAAAKEAAAKEAAAKA

SEQ ID NO: 34 - DNA, 51 bp, unidentified
  source 1..51: mol_type = other DNA
gccgaggccgctgctaaagaggctgccgccaaagaagctgctgccaaggct

SEQ ID NO: 35 - protein, 481 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSKGMSYAMCTGKFKLEKEVAETQHGTILIKVKYEGDGAPCKIPFEIQDVEKKHVNGRLITANPIVTDKESPVNIEAEPPFGDSYIVIGVGDKALKLNWFKKGSSTSASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKSEKDEL

SEQ ID NO: 36 - protein, 578 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPASSTKVDKKIEPRGPTNKPSPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRPTGKSEKDEL

SEQ ID NO: 37 - protein, 594 aa, synthetic construct
MKNTSSLCLLLLVVLCSLTCNSGQATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANAEAAAKEAAAKEAAAKAQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 38 - protein, 578 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSMTEQQWNFAGIEAAASAIQGNVTSIHSLLDEGKQSLTKLAAAWGGSGSEAYQGVQQKWDATATELNNALQNLARTISEAGQAMASTEGNVTGMFAGGGGGMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFTSASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKSEKDEL

SEQ ID NO: 39 - DNA, 1446 bp, synthetic construct
  source 1..1446: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatccaagggcatgtcctacgctatgtgcaccggcaagttcaagttggagaaggaggtggctgagacccagcacggcaccatcttgatcaaggtgaagtacgagggcgatggcgctccttgcaagatccctttcgagatccaggatgtggagaagaagcacgtgaacggaaggttgatcaccgctaaccctatcgtgaccgataaggagtcccctgtgaacatcgaggctgagcctcctttcggcgattcctacatcgtgatcggcgtgggcgataaggctttgaagttgaactggttcaagaagggttcatctactagtgcttccaacacaaaagttgataagaaggttgagccaaagtcctccgataagactcacacttgtccaccatgtccagctccagaacttcttggaggaccatccgttttcttgttcccaccaaagccaaaggatactctcatgatctccaggactccagaggttacatgcgttgtggttgatgtgtctcacgaggatcctgaggtgaagttcaactggtatgtggatggtgttgaggtgcacaacgctaagactaagccacgtgaggaacagtacaactccacttacagggtggtgtctgtgcttactgtgcttcaccaggattggcttaacggcaaagagtacaagtgcaaggtgtccaacaaggctctcccagctccaatcgaaaagactatctccaaggctaagggccagccaagagagccacaagtttacactcttccaccatccagggatgagcttactaagaaccaggtgtcccttacttgcctcgtgaagggattctacccatccgatattgctgttgagtgggagtctaatggccagcctgagaacaactacaagactactccaccagtgctcgattccgatggctcattcttcttgtactccaagctcactgtggataagtccaggtggcagcagggaaacgttttctcttgctctgttatgcacgaggctctccacaaccactacactcagaagtccctctcactctctactggcaagtccgaaaaggatgaactttaa

SEQ ID NO: 40 - DNA, 1737 bp, synthetic construct
  source 1..1737: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatcccagccaaccgaatccattgtgcgcttcccgaacatcaccaatctgtgcccattcggcgaggtgttcaacgccacaagattcgcctctgtgtacgcctggaaccgcaagcgcatctccaattgcgtggccgactactccgtgctctacaactctgccagcttctccacgttcaagtgctacggcgtgtccccgaccaagctgaacgatctctgcttcaccaacgtgtacgccgactccttcgtgatcagaggcgacgaggtgaggcaaattgccccaggccagacaggcaagatcgccgactacaactacaagctcccggacgacttcaccggctgcgtgatcgcttggaactccaacaacctcgactccaaggtcggcggcaactacaattacctctaccgcctcttccgcaagtccaacctcaagccgttcgagcgcgatatctccaccgagatctatcaggccggaagcaccccatgcaatggcgtcgaaggcttcaactgctacttcccgctccagtcctacggcttccagcctacaaatggcgtgggctaccaaccgtacagggtcgtcgttctcagcttcgagctgctccatgctccagccagctctaccaaggtggacaagaagatcgagccaaggggcccgacaaacaagccttctccaccatgcaagtgcccagcgccaaatcttcttggcggcccatccgtgttcatcttcccgccgaagatcaaggacgtgctcatgatctccctctcgccgatcgtgacatgcgtggtggtggatgtgtccgaggacgatccggatgtccagatctcctggttcgtgaacaacgtcgaggtgcacaccgcgcagacacaaacacaccgcgaggactacaatagcaccctcagagtggtgagcgccctgccaatccagcaccaggattggatgtccgggaaagaattcaagtgcaaggtcaacaacaaggacctgccagcgccgatcgagaggaccatctctaagccaaagggctccgtgagagccccgcaagtgtatgttcttccgccgccagaggaagagatgaccaagaagcaagtcaccctgacctgcatggtgaccgacttcatgccagaggatatctacgtcgaatggaccaacaacggcaagaccgagctgaactacaagaacaccgagccggtgctcgactccgacggctcctacttcatgtactccaagctccgcgtcgagaagaagaactgggtcgagcgcaactcctactcctgctccgttgttcatgagggcctccacaaccaccacaccaccaagtctttctcccggccaactggcaagtccgagaaggatgagctttga

SEQ ID NO: 41 - DNA, 1785 bp, synthetic construct
  source 1..1785: mol_type = other DNA
atgaagaatacctccagcctctgcctcctcctcctggtggttctctgctccctcacatgcaattccggccaggccacaccacagaacatcacagatctctgcgccgagtaccacaacacccagatccacacgctcaacgacaagatcttcagctacaccgagagcctcgctggcaagcgcgagatggccattatcaccttcaagaacggcgccaccttccaggttgaggtgccaggctctcagcacatcgactcccagaagaaagccatcgagcgcatgaaggacaccctcaggatcgcctacctcaccgaggccaaggttgagaagctctgcgtgtggaacaacaagaccccgcatgccattgccgccatctctatggccaatgccgaggccgctgctaaagaggctgccgccaaagaagctgctgccaaggctcagccaaccgaatccattgtgcgcttcccgaacatcaccaatctgtgcccattcggcgaggtgttcaacgccacaagattcgcctctgtgtacgcctggaaccgcaagcgcatctccaattgcgtggccgactactccgtgctctacaactctgccagcttctccacgttcaagtgctacggcgtgtccccgaccaagctgaacgatctctgcttcaccaacgtgtacgccgactccttcgtgatcagaggcgacgaggtgaggcaaattgccccaggccagacaggcaagatcgccgactacaactacaagctcccggacgacttcaccggctgcgtgatcgcttggaactccaacaacctcgactccaaggtcggcggcaactacaattacctctaccgcctcttccgcaagtccaacctcaagccgttcgagcgcgatatctccaccgagatctatcaggccggaagcaccccatgcaatggcgtcgaaggcttcaactgctacttcccgctccagtcctacggcttccagcctacaaatggcgtgggctaccaaccgtacagggtcgtcgttctcagcttcgagctgctccatgcgccagcctccaataccaaggtggacaagaaggtcgagccgaagtcctccgacaagacccatacttgcccaccgtgtccagctccagaacttcttggcggcccatccgtgtttctgttcccgccgaagccaaaggacacgctgatgatctctcgcaccccagaggtgacatgcgtggtggtggatgtgtcccacgaggaccctgaggtgaagttcaactggtatgtggacggcgtcgaggtgcacaacgccaagacaaagccgcgcgaggaacagtacaactccacctacagagtggtgtccgtgctcaccgtgctccaccaggattggctgaacgggaaagaatacaaatgcaaggtgtccaacaaggcgctcccagcgccgatcgaaaagaccatctctaaggcgaagggccagccaagagagccacaggtttacacacttccgccgtccagggatgagctgaccaagaaccaggtgtccctgacctgcctcgtgaagggcttctacccatccgatatcgccgtcgagtgggagtccaatggccagcctgagaacaattacaagaccacaccgccggtgctcgactccgatggctcattcttcctgtactccaagctgaccgtcgacaaatccagatggcaacagggcaacgtgttctcctgctccgttatgcacgaggccctccacaaccactacacccagaagtctctcagcctctcgccaggcaagtccgagaaggatgagctttga

SEQ ID NO: 42 - DNA, 1737 bp, synthetic construct
  source 1..1737: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatccatgactgagcagcagtggaattttgctggtattgaggctgctgcttctgctattcagggtaatgttacttctattcattctcttcttgatgagggtaaacagtctcttactaagttggctgctgcttggggtggttctggttctgaggcttaccagggtgttcagcagaagtgggatgctactgctactgagcttaacaatgctcttcagaatcttgctaggactatttctgaggctggtcaggctatggcttctactgagggtaatgttactggtatgtttgctggtggtggtggtggtatggctgagatgaagactgatgctgctactcttgctcaggaggctggtaattttgagaggatttctggagatttgaagactcagattgatcaggttgagtctactgctggttctcttcagggtcagtggaggggtgctgctggtactgctgctcaggctgctgttgttaggtttcaggaggctgctaataagcagaagcaggagcttgatgagatttctactaatattaggcaggctggtgttcagtactctagggctgatgaggagcagcagcaggctctttcttctcagatgggttttactagtgcttccaacacaaaagttgataagaaggttgagccaaagtcctccgataagactcacacttgtccaccatgtccagctccagaacttcttggaggaccatccgttttcttgttcccaccaaagccaaaggatactctcatgatctccaggactccagaggttacatgcgttgtggttgatgtgtctcacgaggatcctgaggtgaagttcaactggtatgtggatggtgttgaggtgcacaacgctaagactaagccacgtgaggaacagtacaactccacttacagggtggtgtctgtgcttactgtgcttcaccaggattggcttaacggcaaagagtacaagtgcaaggtgtccaacaaggctctcccagctccaatcgaaaagactatctccaaggctaagggccagccaagagagccacaagtttacactcttccaccatccagggatgagcttactaagaaccaggtgtcccttacttgcctcgtgaagggattctacccatccgatattgctgttgagtgggagtctaatggccagcctgagaacaactacaagactactccaccagtgctcgattccgatggctcattcttcttgtactccaagctcactgtggataagtccaggtggcagcagggaaacgttttctcttgctctgttatgcacgaggctctccacaaccactacactcagaagtccctctcactctctactggcaagtccgaaaaggatgaactttaa

SEQ ID NO: 43 - protein, 16 aa, Mus musculus
EPRGPTIKPSPPCKCP

SEQ ID NO: 44 - protein, 15 aa, Homo sapiens
EPKSCDKTHTCPPCP

SEQ ID NO: 45 - protein, 18 aa, Homo sapiens
PTLYNVSLVMSDTAGTCY

SEQ ID NO: 46 - DNA, 54 bp, Homo sapiens
  source 1..54: mol_type = other DNA
ccaactctctacaacgtgtccctcgtgatgtctgatactgctggcacttgctac

SEQ ID NO: 47 - protein, 499 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSKGMSYAMCTGKFKLEKEVAETQHGTILIKVKYEGDGAPCKIPFEIQDVEKKHVNGRLITANPIVTDKESPVNIEAEPPFGDSYIVIGVGDKALKLNWFKKGSSTSASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKPTLYNVSLVMSDTAGTCYSEKDEL

SEQ ID NO: 48 - protein, 596 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPASSTKVDKKIEPRGPTNKPSPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRPTGKPTLYNVSLVMSDTAGTCYSEKDEL

SEQ ID NO: 49 - protein, 612 aa, synthetic construct
MKNTSSLCLLLLVVLCSLTCNSGQATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANAEAAAKEAAAKEAAAKAQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCYSEKDEL

SEQ ID NO: 50 - protein, 596 aa, synthetic construct
MELGLSWIFLLAILKGVQCTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPGSMTEQQWNFAGIEAAASAIQGNVTSIHSLLDEGKQSLTKLAAAWGGSGSEAYQGVQQKWDATATELNNALQNLARTISEAGQAMASTEGNVTGMFAGGGGGMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFTSASNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKPTLYNVSLVMSDTAGTCYSEKDEL

SEQ ID NO: 51 - DNA, 1500 bp, synthetic construct
  source 1..1500: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatccaagggcatgtcctacgctatgtgcaccggcaagttcaagttggagaaggaggtggctgagacccagcacggcaccatcttgatcaaggtgaagtacgagggcgatggcgctccttgcaagatccctttcgagatccaggatgtggagaagaagcacgtgaacggaaggttgatcaccgctaaccctatcgtgaccgataaggagtcccctgtgaacatcgaggctgagcctcctttcggcgattcctacatcgtgatcggcgtgggcgataaggctttgaagttgaactggttcaagaagggttcatctactagtgcttccaacacaaaagttgataagaaggttgagccaaagtcctccgataagactcacacttgtccaccatgtccagctccagaacttcttggaggaccatccgttttcttgttcccaccaaagccaaaggatactctcatgatctccaggactccagaggttacatgcgttgtggttgatgtgtctcacgaggatcctgaggtgaagttcaactggtatgtggatggtgttgaggtgcacaacgctaagactaagccacgtgaggaacagtacaactccacttacagggtggtgtctgtgcttactgtgcttcaccaggattggcttaacggcaaagagtacaagtgcaaggtgtccaacaaggctctcccagctccaatcgaaaagactatctccaaggctaagggccagccaagagagccacaagtttacactcttccaccatccagggatgagcttactaagaaccaggtgtcccttacttgcctcgtgaagggattctacccatccgatattgctgttgagtgggagtctaatggccagcctgagaacaactacaagactactccaccagtgctcgattccgatggctcattcttcttgtactccaagctcactgtggataagtccaggtggcagcagggaaacgttttctcttgctctgttatgcacgaggctctccacaaccactacactcagaagtccctctcactctctactggcaagccaactctctacaacgtgtccctcgtgatgtctgatactgctggcacttgctactccgaaaaggatgaactttaa

SEQ ID NO: 52 - DNA, 1791 bp, synthetic construct
  source 1..1791: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatcccagccaaccgaatccattgtgcgcttcccgaacatcaccaatctgtgcccattcggcgaggtgttcaacgccacaagattcgcctctgtgtacgcctggaaccgcaagcgcatctccaattgcgtggccgactactccgtgctctacaactctgccagcttctccacgttcaagtgctacggcgtgtccccgaccaagctgaacgatctctgcttcaccaacgtgtacgccgactccttcgtgatcagaggcgacgaggtgaggcaaattgccccaggccagacaggcaagatcgccgactacaactacaagctcccggacgacttcaccggctgcgtgatcgcttggaactccaacaacctcgactccaaggtcggcggcaactacaattacctctaccgcctcttccgcaagtccaacctcaagccgttcgagcgcgatatctccaccgagatctatcaggccggaagcaccccatgcaatggcgtcgaaggcttcaactgctacttcccgctccagtcctacggcttccagcctacaaatggcgtgggctaccaaccgtacagggtcgtcgttctcagcttcgagctgctccatgctccagccagctctaccaaggtggacaagaagatcgagccaaggggcccgacaaacaagccttctccaccatgcaagtgcccagcgccaaatcttcttggcggcccatccgtgttcatcttcccgccgaagatcaaggacgtgctcatgatctccctctcgccgatcgtgacatgcgtggtggtggatgtgtccgaggacgatccggatgtccagatctcctggttcgtgaacaacgtcgaggtgcacaccgcgcagacacaaacacaccgcgaggactacaatagcaccctcagagtggtgagcgccctgccaatccagcaccaggattggatgtccgggaaagaattcaagtgcaaggtcaacaacaaggacctgccagcgccgatcgagaggaccatctctaagccaaagggctccgtgagagccccgcaagtgtatgttcttccgccgccagaggaagagatgaccaagaagcaagtcaccctgacctgcatggtgaccgacttcatgccagaggatatctacgtcgaatggaccaacaacggcaagaccgagctgaactacaagaacaccgagccggtgctcgactccgacggctcctacttcatgtactccaagctccgcgtcgagaagaagaactgggtcgagcgcaactcctactcctgctccgttgttcatgagggcctccacaaccaccacaccaccaagtctttctcccggccaactggcaagccaactctctacaacgtgtccctcgtgatgtctgatactgctggcacttgctactccgagaaggatgagctttga

SEQ ID NO: 53 - DNA, 1839 bp, synthetic construct
  source 1..1839: mol_type = other DNA
atgaagaatacctccagcctctgcctcctcctcctggtggttctctgctccctcacatgcaattccggccaggccacaccacagaacatcacagatctctgcgccgagtaccacaacacccagatccacacgctcaacgacaagatcttcagctacaccgagagcctcgctggcaagcgcgagatggccattatcaccttcaagaacggcgccaccttccaggttgaggtgccaggctctcagcacatcgactcccagaagaaagccatcgagcgcatgaaggacaccctcaggatcgcctacctcaccgaggccaaggttgagaagctctgcgtgtggaacaacaagaccccgcatgccattgccgccatctctatggccaatgccgaggccgctgctaaagaggctgccgccaaagaagctgctgccaaggctcagccaaccgaatccattgtgcgcttcccgaacatcaccaatctgtgcccattcggcgaggtgttcaacgccacaagattcgcctctgtgtacgcctggaaccgcaagcgcatctccaattgcgtggccgactactccgtgctctacaactctgccagcttctccacgttcaagtgctacggcgtgtccccgaccaagctgaacgatctctgcttcaccaacgtgtacgccgactccttcgtgatcagaggcgacgaggtgaggcaaattgccccaggccagacaggcaagatcgccgactacaactacaagctcccggacgacttcaccggctgcgtgatcgcttggaactccaacaacctcgactccaaggtcggcggcaactacaattacctctaccgcctcttccgcaagtccaacctcaagccgttcgagcgcgatatctccaccgagatctatcaggccggaagcaccccatgcaatggcgtcgaaggcttcaactgctacttcccgctccagtcctacggcttccagcctacaaatggcgtgggctaccaaccgtacagggtcgtcgttctcagcttcgagctgctccatgcgccagcctccaataccaaggtggacaagaaggtcgagccgaagtcctccgacaagacccatacttgcccaccgtgtccagctccagaacttcttggcggcccatccgtgtttctgttcccgccgaagccaaaggacacgctgatgatctctcgcaccccagaggtgacatgcgtggtggtggatgtgtcccacgaggaccctgaggtgaagttcaactggtatgtggacggcgtcgaggtgcacaacgccaagacaaagccgcgcgaggaacagtacaactccacctacagagtggtgtccgtgctcaccgtgctccaccaggattggctgaacgggaaagaatacaaatgcaaggtgtccaacaaggcgctcccagcgccgatcgaaaagaccatctctaaggcgaagggccagccaagagagccacaggtttacacacttccgccgtccagggatgagctgaccaagaaccaggtgtccctgacctgcctcgtgaagggcttctacccatccgatatcgccgtcgagtgggagtccaatggccagcctgagaacaattacaagaccacaccgccggtgctcgactccgatggctcattcttcctgtactccaagctgaccgtcgacaaatccagatggcaacagggcaacgtgttctcctgctccgttatgcacgaggccctccacaaccactacacccagaagtctctcagcctctcgccaggcaagccaactctctacaacgtgtccctcgtgatgtctgatactgctggcacttgctactccgagaaggatgagctttga

SEQ ID NO: 54 - DNA, 1790 bp, synthetic construct
  source 1..1790: mol_type = other DNA
atggaactcggcctttcttggattttcttgctcgctatcctcaagggcgtgcaatgcacacctcaaaatattactgatttgtgtgcagaataccacaacacacaaatacatacgctaaatgataagatattttcgtatacagaatctctagctggaaaaagagagatggctatcattacttttaagaatggtgcgacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaatggcccaggcccgggatccatgactgagcagcagtggaattttgctggtattgaggctgctgcttctgctattcagggtaatgttacttctattcattctcttcttgatgagggtaaacagtctcttactaagttggctgctgcttggggtggttctggttctgaggcttaccagggtgttcagcagaagtgggatgctactgctactgagcttaacaatgctcttcagaatcttgctaggactatttctgaggctggtcaggctatggcttctactgagggtaatgttactggtatgtttgctggtggtggtggtggtatggctgagatgaagactgatgctgctactcttgctcaggaggctggtaattttgagaggatttctggagatttgaagactcagattgatcaggttgagtctactgctggttctcttcagggtcagtggaggggtgctgctggtactgctgctcaggctgctgttgttaggtttcaggaggctgctaataagcagaagcaggagcttgatgagatttctactaatattaggcaggctggtgttcagtactctagggctgatgaggagcagcagcaggctctttcttctcagatgggttttactagtgcttccaacacaaaagttgataagaaggttgagccaaagtcctccgataagactcacacttgtccaccatgtccagctccagaacttcttggaggaccatccgttttcttgttcccaccaaagccaaaggatactctcatgatctccaggactccagaggttacatgcgttgtggttgatgtgtctcacgaggatcctgaggtgaagttcaactggtatgtggatggtgttgaggtgcacaacgctaagactaagccacgtgaggaacagtacaactccacttacagggtggtgtctgtgcttactgtgcttcaccaggattggcttaacggcaaagagtacaagtgcaaggtgtccaacaaggctctcccagctccaatcgaaaagactatctccaaggctaagggccagccaagagagccacaagtttacactcttccaccatccagggatgagcttactaagaaccaggtgtcccttacttgcctcgtgaagggattctacccatccgatattgctgttgagtgggagtctaatggccagcctgagaacaactacaagactactccaccagtgctcgattccgatggctcattcttcttgtactccaagctcactgtggataagtccaggtggcagcagggaaacgttttctcttgctctgttatgcacgaggctctccacaaccactacactcagaagtccctctcactctctactggcaagccaactctctacaacgtgtccctcgtgatgtctgatactgctggcacttgctatccgaaaaggatgaactttaa

(19) United States
(12) Patent Application Publication
(10) Pub. No.: US 2025/0127888 A1
(43) Pub. Date: Apr. 24, 2025

(54) Immunogenic Fusion Peptides Including FC Fragment and a Non Toxic B Subunit of AB5 Toxin

(71) Applicants: St George's Hospital Medical School, London (GB); Jeonbuk National University, Jeollabuk-do (KR); Genecell Biotech Inc., Jeollabuk-do (GB)

(72) Inventors: Rajko Reljic, London (GB); Mi-Young Kim, London (GB); Moon-Sik Yang, Jeollabuk-do (KR); Yong-Suk Jang, Jeollabuk-do (KR); Tae-Ho Kwon, Jeollabuk-do (KR)

(21) Appl. No.: 18/693,382

(22) PCT Filed: Sep. 23, 2022
(86) PCT No.: PCT/GB2022/052418

§ 371 Date: Mar. 19, 2024

(30) Foreign Application Priority Data
Sep. 23, 2021 (GB) 2113559.5

(51) Int. Cl. A61K 39/385; A61K 9/00; A61K 39/00; A61K 39/215; A61P 31/14; C07K 14/245; C07K 14/25; C07K 14/28; C12N 7/00

(52) U.S. Cl. CPC A61K 39/385; A61K 9/0043; A61K 39/215; A61P 31/14; C07K 14/245; C07K 14/25; C07K 14/28; C12N 7/00; A61K 2039/542; A61K 2039/575; A61K 2039/6037; C07K 2319/02; C07K 2319/30; C07K 2319/55; C12N 2770/20034

(57) ABSTRACT

The invention relates to fusion polypeptides, and to immunogenic polypeptides and their use as vaccines for treating, preventing or ameliorating a wide range of infectious diseases, for example caused by a virus, bacterium or fungus, or cancer. The fusion polypeptide comprises: a first amino acid sequence comprising a non-toxic B subunit of an AB₅ Toxin, or a fragment or variant thereof; a second amino acid sequence comprising an immunoglobulin Fc region (Ig-Fc), or a fragment or variant thereof; and a third amino acid sequence comprising an antigen, or a fragment or variant thereof. The invention also extends to nucleic acids encoding such fusion polypeptides and proteins, and to recombinant vectors expressing such nucleic acids. The invention is especially useful for the rapid development of protein-based vaccines, and to their use in methods of treating infectious diseases or cancer, and also to pharmaceutical compositions comprising the fusion proteins.

The present invention relates to fusion polypeptides, and particularly, although not exclusively, to immunogenic polypeptides and their use as vaccines for treating, preventing or ameliorating a wide range of infectious diseases, for example caused by a virus, bacterium or fungus, or cancer. The invention also extends to nucleic acids encoding such fusion polypeptides and proteins, and to recombinant vectors expressing such nucleic acids. The invention is especially useful for the rapid development of protein-based vaccines, and to their use in methods of treating infectious diseases or cancer, and also to pharmaceutical compositions comprising the fusion proteins.

Vaccine development against human and animal respiratory diseases, such as Severe Acute Respiratory Syndrome (SARS), Middle East Respiratory Syndrome (MERS), Severe acute respiratory syndrome coronavirus 2 (SARS-CoV2), Influenza, Whooping Cough, Tuberculosis, PED (Porcine Epidemic Diarrhoea), Swine Fever (SF), Transmissible Gastroenteritis (TGE) and Swine Flu (influenza), is of paramount importance, in order to arrest major pandemics and prevent future outbreaks.

Several vaccine ‘subunit’ platform technologies have been developed over the years, including nucleic acids, viral vectors and protein with adjuvants, all contributing to combating emerging infectious diseases worldwide. However, each of these different vaccine platforms present distinct advantages and disadvantages. Recently, the use of RNA-based vaccines against SARS-Cov2 pandemic has come into focus, delivering remarkable results in a short time span. These vaccines present significant advantages over the classical killed/live/attenuated vaccine approach. For example, for SARS-CoV-2, large quantities of virus would need to be grown under biosafety level 3 (BSL3) conditions for a whole-inactivated vaccine; extensive safety testing is required to ensure live-attenuated viruses are safe and do not easily revert to wild type, and several recombinant proteins need to be produced simultaneously for virus-like particle (VLP) type vaccines. However, RNA-based vaccines are not easily adapted to cover emerging pathogen variants. Besides RNA-based vaccines, all other licensed subunit vaccines are based on viral vectors or adjuvanted proteins, and present the same limitations.

With the emergence of new variants, not covered by the existing licensed vaccines, novel vaccine platforms should enable rapid and easy development, production and regulatory approval to cover new variants in an ever-changing pathogen landscape to ensure successful approaches to immunisation against infectious diseases.

Therefore, to overcome the limitations of the previous vaccine platforms, the inventors have created and tested a novel, fully protein vaccine platform, which involves an immunogenic fusion polypeptide (or protein) incorporating the non-toxic B subunit of cholera toxin (CTB) and an immunoglobulin Fc (Ig-Fc) region with dual adjuvant activity, that allows easy incorporation of any antigen with these two molecules. Because of the importance on CTB and Fc, this vaccine platform technology has been called PCF (i.e. Platform CTB-Fc). The PCF platform can be used for mucosal and systemic vaccine delivery, and is composed of three primary components combined in a single polypeptide chain.

However, it will be appreciated that the CTB is only an exemplary non-toxic B subunit of an AB₅ toxin, and that any other AB₅ toxin B subunit may be used in the immunogenic fusion polypeptide of the present invention.

Accordingly, in a first aspect of the invention, there is provided a fusion polypeptide comprising:
(i) a first amino acid sequence comprising a non-toxic B subunit of an AB₅ Toxin, or a fragment or variant thereof;
(ii) a second amino acid sequence comprising an immunoglobulin Fc region, or a fragment or variant thereof; and
(iii) a third amino acid sequence comprising an antigen, or a fragment or variant thereof.

The fusion polypeptide is preferably immunogenic.

Advantageously, and preferably, immune cells target the antigen, or fragment or variant thereof, through the Ig-Fc, thereby enhancing its uptake. Preferably, and advantageously, the antigen, or a fragment or variant thereof, is delivered directly to Fc-receptor-bearing antigen-presenting cells (APCs) in the context of a strong AB₅ Toxin-mediated mucosal immune response, for example cholera toxin B subunit (CTB). Accordingly, the fusion polypeptide is, therefore, a highly innovative vaccine approach that is designed to induce a fast-acting robust mucosal immune response preferably in the respiratory and/or gut mucosae, which are key target organs for many types of infection. Thus, preferably the fusion polypeptide is mucosally administrable, preferably intranasally.

For example, SARS-CoV2, which is causing the Covid-19 pandemic, is a respiratory pathogen, and so it is likely that a robust mucosal immune response in the lungs and the upper respiratory tract will be important for protection. Furthermore, advantageously, the aerosolised delivery of the polypeptide vaccine by inhalation would be particularly amenable for large vaccination campaigns in a relatively short space of time, due to its ease of administration and the reduced need for needle-trained clinical personnel. Moreover, advantageously, the vaccine platform is suitable for both initial immunization, or as a mucosal boost to another (likely) systemic vaccine.

Although many preclinical vaccine candidates, as well as a number of licensed vaccines, have been developed for SARS-CoV2, very few of them are designed for mucosal application. Moreover, the majority of protein subunit candidates listed on the WHO website are based on the S protein of SARS-CoV2, but no information is given about the required adjuvants. Thus, adjuvant requirement is a major bottleneck in vaccine development. However, in contrast, the fusion polypeptide of the invention has a built-in molecular adjuvanticity through the AB₅ Toxin adjuvant (e.g. CTB), and so it is in effect a self-adjuvanting vaccine. Therefore, the polypeptide provides a solution to the vaccine reliance on exogenous adjuvants and instead generates its own adjuvanticity by molecular mechanisms made possible by the novel structural and functional components of the fusion. As such, there is no adjuvant restriction using the polypeptide of the invention.

The polypeptide also combines advantages of classical and novel vaccine platforms, including the provision of a fully polypeptide or protein-based vaccine, which, therefore, has fewer safety concerns. In addition, the fusion polypeptide of the invention provides several distinct advantages over other vaccine platforms, including simplicity of production and formulation (based on a single polypeptide or protein), suitability for mucosal application and a potential for a large-scale, cost-effective production in expression systems, such as plants. The protein-only nature of the vaccine is optimal for good manufacturing practice (GMP) production and human application.

In one embodiment, the fusion polypeptide preferably comprises first, second and third amino acid sequences (or domains), respectively: (i) an AB₅ toxin B subunit, or a fragment or variant thereof; (ii) an immunoglobulin Fc region, or a fragment or variant thereof (Ig-Fc); and (iii) an antigen, or a fragment or variant thereof.

These three amino acid sequences or domains may be disposed in any order in the fusion polypeptide from the N-terminus to the C-terminus. For example, the order in the N- to C-direction, may be (i), (ii), and (iii). Alternatively, the order in the N- to C-direction, may be (iii), (ii), and (i). Alternatively, the order in the N- to C-direction, may be (ii), (i), and (iii). Alternatively, the order in the N- to C-direction, may be (ii), (iii), and (i). Alternatively, the order in the N- to C-direction, may be (iii), (i), and (ii).

For example, in one embodiment, the second amino acid sequence comprising the Ig-Fc region, or a fragment or variant thereof, may be disposed at or towards the N-terminus of the fusion polypeptide, the first amino acid comprising the AB₅ toxin B subunit, or a fragment or variant thereof, may be disposed at or towards the C-terminus of the polypeptide, and the third amino acid sequence comprising the antigen, or a fragment of variant thereof, may be disposed in between the first and second amino acid sequences.

In other embodiments, the position of the third amino acid comprising the antigen or fragment or variant thereof may be disposed at or towards either the N-terminus, or the C-terminus, of the fusion polypeptide.

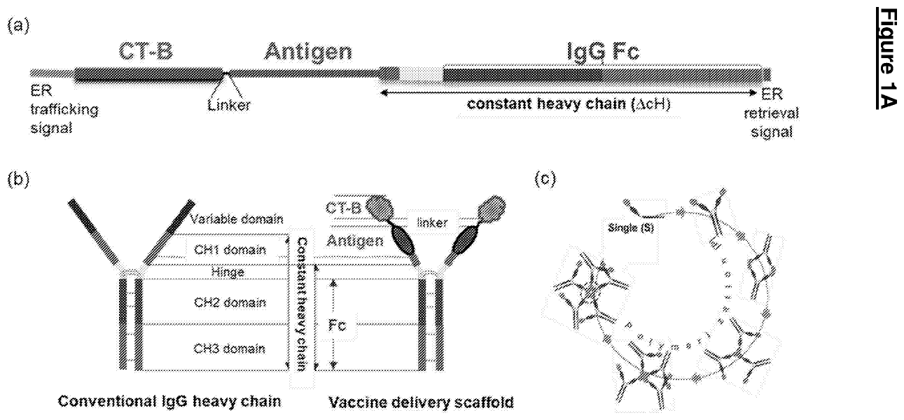

However, in a preferred embodiment, the first amino acid sequence comprising the AB₅ toxin B subunit, or a fragment or variant thereof, is disposed at or towards the N-terminus of the fusion polypeptide, and the second amino acid sequence comprising the Ig-Fc region, or a fragment or variant thereof is disposed at or towards the C-terminus of the polypeptide, and the third amino acid sequence comprising the antigen, or a fragment or variant thereof, is disposed in between the first and second amino acid sequences. This preferred embodiment is shown in FIG. 1(a).

Therefore, preferably the first amino acid sequence comprising the AB₅ toxin B subunit or a fragment or variant thereof is disposed at or towards the N-terminus of the fusion polypeptide.

The AB₅ toxins are known to the skilled person and consist of six-component protein complexes secreted by certain pathogenic bacteria known to cause human diseases, such as cholera, dysentery, and haemolytic-uraemic syndrome. One component is known as an “A subunit”, and the remaining five components are “B subunits”. These toxins share a similar structure and mechanism for entering targeted host cells. The B subunit is responsible for binding to receptors to open up a pathway for the A subunit to enter the cell.

Accordingly, in one embodiment, the AB₅ toxin B subunit in the fusion polypeptides of the invention is a haemolytic-uraemic B subunit. The haemolytic-uraemic B subunit may be represented by Escherichia coli's heat labile enterotoxin (LT) subunit B.

In another embodiment, the AB₅ toxin B subunit is a dysenteric B subunit. The dysenteric B subunit may be represented by Shigella dysenteriae's shiga toxin (Stx) subunit B.

In a preferred embodiment, however, the AB₅ toxin B subunit is a cholera toxin B subunit (CTB). The cholera toxin B subunit may be represented by Vibrio cholerae's cholera toxin subunit B. The non-toxic CTB is a known and licensed adjuvant, and has already been proven to be safe for use in many vaccine compositions. Advantageously, therefore, the CTB component enables the self-adjuvanting properties of the fusion polypeptide of the invention.

The B-pentamer of the cholera toxin (CTB) or of the Escherichia coli heat-labile enterotoxin (LT) binds specifically to the branched pentasaccharide moiety of ganglioside GM1 (accession number: 1EEI_D), which has been found to be selectively expressed in the surface cell membrane of a variety of cell types, particularly in nervous tissue. The saccharide moiety of GM1 is bound by the complete AB₅ hexamer and also by the B-pentamer, but not by monomeric B subunits (De Wolf et al., 1981). Although the normal function of the gangliosides is so far poorly understood, they have recently been implicated in various signal transduction pathways. Exogenous GM1 affects Ca2+ signaling (Hilbush & Levine, 1992), modulation of CD4 expression (Morrison et al., 1991), and modulation of the tyrosine-protein kinase activity of epidermal growth factor (Weis & Davis, 1990). The cholera toxin B subunit itself is known to interfere with or potentiate the effect of several growth factors.

Hence, in one embodiment, the amino acid sequence of the CTB component (which may be for a GM1 ganglioside-targeting domain) is referred to herein as SEQ ID No: 1, as follows:

[SEQ ID No: 1]
TPQNITDLCAEYHNTQIHTLNDKIFSYTESLAGKREMAIITFKNGATFQ
VEVPGSQHIDSQKKAIERMKDTLRIAYLTEAKVEKLCVWNNKTPHAIAA
ISMAN

Therefore, preferably the first amino acid sequence (i.e. comprising CTB) comprises or consists of an amino acid sequence substantially as set out in SEQ ID No: 1, or a fragment or variant thereof.

In one embodiment, the nucleic acid sequence encoding the CTB component (which may be plant-codon optimised) is referred to herein as SEQ ID No: 2, as follows:

[SEQ ID No: 2]
ACACCACAGAACATCACAGATCTCTGCGCCGAGTACCACAACACCCAGATCCACACGCTCAACGACAAGATCTTCAGC
TACACCGAGAGCCTCGCTGGCAAGCGCGAGATGGCCATTATCACCTTCAAGAACGGCGCCACCTTCCAGGTTGAGGTG
CCAGGCTCTCAGCACATCGACTCCCAGAAGAAAGCCATCGAGCGCATGAAGGACACCCTCAGGATCGCCTACCTCACC
GAGGCCAAGGTTGAGAAGCTCTGCGTGTGGAACAACAAGACCCCGCATGCCATTGCCGCCATCTCTATGGCCAAT

Therefore, preferably the first amino acid sequence (i.e. comprising CTB) is encoded by a nucleotide sequence substantially as set out in SEQ ID No:2, or a fragment or variant thereof.

Preferably, the second amino acid sequence comprising the Ig-Fc region, or a fragment or variant thereof, is disposed at or towards the C-terminus of the fusion polypeptide. Advantageously, the Ig-Fc component also contributes to the self-adjuvanting properties of the fusion polypeptide.

Preferably, the Ig-Fc region, or a fragment or variant thereof, is either derived from, or of, human or animal origin.

The skilled person will know that the fragment crystallizable region (Fc region) of an immunoglobulin is the tail region of an antibody that interacts with cell surface receptors called Fc receptors, and some proteins of the complement system. This property allows antibodies to activate the immune system. In IgG, IgA and IgD antibody isotypes, the Fc region is composed of two identical protein fragments, derived from the second and third constant domains of the antibody's two heavy chains, i.e. CH₂—CH₃. In IgM and IgE, Fc regions contain three heavy chain constant domains (CH domains 2-4) in each polypeptide chain. The Fc regions of IgGs bear a highly conserved N-glycosylation site, and glycosylation of the Fc fragment is believed to be important for Fc receptor-mediated activity. The N-glycans attached to this N-glycosylation site are predominantly core-fucosylated diantennary structures of the complex type. In addition, small amounts of these N-glycans also bear bisecting GlcNAc and α-2,6 linked sialic acid residues.

In one embodiment, the glycan moiety is formed by two N-linked biantennary oligosaccharide chains consisting of a core heptasaccharide [N-acetylglucosamine (GlcNAc) and mannose (Man)], but the occurrence of other residues, such as terminal N-acetyl neuraminic acid, galactose (Gal), bisecting N-acetylglucosamine (GlcNAc), and fucose (Fuc) have also been reported. Additionally, 5-17 and 2-7% of IgG structures could be monosialylated and disialylated, respectively. This imparts a significant complexity and heterogeneity to therapeutic IgG molecules when expressed in mammalian cells, which can affect the therapeutic profile of IgG.

Accordingly, in a preferred embodiment, the fusion polypeptide may be tagged with an ER-retention signal peptide, which results in it having predominantly an ER-type oligomannosidic (Man8) and some Golgi-processed complex N-glycans (GnGn). This configuration provides an additional advantage in that more uniformed glycans are present in the fusion polypeptide, rather than N-linked biantennary complex-type oligosaccharides. In some embodiments, the ER-retention signal peptide may comprise an amino acid sequence substantially as set out in SEQ ID No: 29, which is described herein.

In an embodiment, the Ig-Fc region, or a fragment or variant thereof may be selected from IgA, IgD, IgE, IgG and/or IgM. For each Ig-Fc region, or a fragment or variant thereof, all isotypes are considered. For example, for IgG, the isotypes IgG1, IgG2, IgG3, IgG4 can be used as the Ig-Fc region, or a fragment or variant thereof in the fusion polypeptide.

In a preferred embodiment, however, the Ig-Fc region, or a fragment or variant thereof is an IgG. Preferably, the IgG is a human IgG, more preferably a human IgG1. Preferably, the IgG is a mouse IgG, more preferably a mouse IgG2a.

One potential limitation associated with using CTB in polypeptide constructs is that the monomeric 11.5 kDa CTB cannot efficiently polymerise (i.e. pentamerise) once fused to another protein. To overcome this limitation, therefore, the inventors have developed an elegant linker system in the fusion polypeptide of the first aspect, which comprises a CH domain of an immunoglobulin, or a portion thereof, that enables the formation of pentameric IgM-size PCF molecules.

Therefore, preferably the Ig-Fc region, or a fragment or variant thereof comprises a CH domain of an immunoglobulin. The inclusion of the CH domain is surprisingly effective in harnessing the dual functionality of the fusion polypeptide in vivo, that is, the self-adjuvanting properties provided by both CTB and Fc.

Furthermore, antigens larger than the 25 kDa IgG Fab fragment (i.e. the remaining part of the antibody) could obstruct the Fc receptor binding function (also known as steric hindrance). Therefore, including a hinge region alone may not be sufficient to avoid steric hindrance in some embodiments. Hence, the novel inclusion of an immunoglobulin CH domain or a portion thereof mitigates this issue, and the inventors have successfully demonstrated that the PCFs of the invention comprising larger antigens, such as Dengue (>26 kDa), SARS-CoV2 (>40 kDa) and TB (>36 kDa), displayed efficient binding of the Fc to antigen presenting cells (APCs).

Preferably, the Ig-Fc region, or a fragment or variant thereof comprises a CH₃ domain of an immunoglobulin. The CH₃ domain may be from a mouse, preferably IgG2a. In one embodiment, the amino acid sequence of the CH₃ domain (mouse IgG2a) is referred to herein as SEQ ID No: 3, as follows:

[SEQ ID No: 3]
GSVRAPQVYVLPPPEEEMTKKQVTLTCMVTDFMPEDIYVEWTNNGKTEL
NYKNTEPVLDSDGSYFMYSKLRVEKKNWVERNSYSCSVVHEGLHNHHTT
KSFSRPTGK

Therefore, preferably the second amino acid sequence (i.e. comprising the Ig-Fc region) comprises or consists of an amino acid sequence substantially as set out in SEQ ID No: 3, or a fragment or variant thereof.

In one embodiment, the nucleic acid sequence (which may be plant-codon optimised) encoding the CH₃ domain (mouse IgG2a) is referred to herein as SEQ ID No: 4, as follows:

[SEQ ID No: 4]
GGCTCCGTGAGAGCCCCGCAAGTGTATGTTCTTCCGCCGCCAGAGGAAGAGATGACCAAGAAGCAAGTCACCCTGACC
TGCATGGTGACCGACTTCATGCCAGAGGATATCTACGTCGAATGGACCAACAACGGCAAGACCGAGCTGAACTACAAG
AACACCGAGCCGGTGCTCGACTCCGACGGCTCCTACTTCATGTACTCCAAGCTCCGCGTCGAGAAGAAGAACTGGGTC
GAGCGCAACTCCTACTCCTGCTCCGTTGTTCATGAGGGCCTCCACAACCACCACACCACCAAGTCTTTCTCCCGGCCA
ACTGGCAAG

Therefore, preferably the second amino acid sequence (i.e. comprising the Ig-Fc region) is encoded by a nucleotide sequence substantially as set out in SEQ ID No: 4, or a fragment or variant thereof.

The CH₃ domain may be from a human, preferably IgG1. In one embodiment, the amino acid sequence of the CH₃ domain (human IgG1) is referred to herein as SEQ ID No: 5, as follows:

[SEQ ID No: 5]
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Therefore, preferably the second amino acid sequence (i.e. comprising the Ig-Fc region) comprises or consists of an amino acid sequence substantially as set out in SEQ ID No: 5, or a fragment or variant thereof.

In one embodiment, the nucleic acid sequence (which may be plant-codon optimised) encoding the CH₃ domain (human IgG1) is referred to herein as SEQ ID No: 6, as follows:

[SEQ ID No: 6]
GGCCAGCCAAGAGAGCCACAGGTTTACACACTTCCGCCGTCCAGGGATGAGCTGACCAAGAACCAGGTGTCCCTGACC
TGCCTCGTGAAGGGCTTCTACCCATCCGATATCGCCGTCGAGTGGGAGTCCAATGGCCAGCCTGAGAACAATTACAAG
ACCACACCGCCGGTGCTCGACTCCGATGGCTCATTCTTCCTGTACTCCAAGCTGACCGTCGACAAATCCAGATGGCAA
CAGGGCAACGTGTTCTCCTGCTCCGTTATGCACGAGGCCCTCCACAACCACTACACCCAGAAGTCTCTCAGCCTCTCG
CCAGGCAAG

Therefore, preferably the second amino acid sequence (i.e. comprising the Ig-Fc region) is encoded by a nucleotide sequence substantially as set out in SEQ ID No: 6, or a fragment or variant thereof.

Preferably, the Ig-Fc region, or a fragment or variant thereof comprises a CH₂ domain of an immunoglobulin. The CH₂ domain may be from a mouse, preferably IgG2a. In one embodiment, the amino acid sequence of the CH₂ domain (mouse IgG2a) is referred to herein as SEQ ID No: 7, as follows:

APNLLGGPSVFIFPPKIKDVLMISLSPIVTCVVVDVSEDDPDVQISW
FVNNVEVHTAQTQTHREDYNSTLRVVSALPIQHQDWMSGKEFKCKVN
NKDLPAPIERTISKPK

Therefore, preferably the second amino acid sequence (i.e. comprising the Ig-Fc region) comprises or consists of an amino acid sequence substantially as set out in SEQ ID No: 7, or a fragment or variant thereof.

In one embodiment, the nucleic acid sequence (which may be plant-codon optimised) encoding the CH₂ domain (mouse IgG2a) is referred to herein as SEQ ID No: 8, as follows:

[SEQ ID No: 8]
GCGCCAAATCTTCTTGGCGGCCCATCCGTGTTCATCTTCCCGCCGAAGATCAAGGACGTGCTCATGATCTCCCTCTCG
CCGATCGTGACATGCGTGGTGGTGGATGTGTCCGAGGACGATCCGGATGTCCAGATCTCCTGGTTCGTGAACAACGTC
GAGGTGCACACCGCGCAGACACAAACACACCGCGAGGACTACAATAGCACCCTCAGAGTGGTGAGCGCCCTGCCAATC
CAGCACCAGGATTGGATGTCCGGGAAAGAATTCAAGTGCAAGGTCAACAACAAGGACCTGCCAGCGCCGATCGAGAGG
ACCATCTCTAAGCCAAAG

Therefore, preferably the second amino acid sequence (i.e. comprising the Ig-Fc region) is encoded by a nucleotide sequence substantially as set out in SEQ ID No:8, or a fragment or variant thereof.

The CH₂ domain may be from a human, preferably IgG1. In one embodiment, the amino acid sequence of the CH₂ domain (human IgG1) is referred to herein as SEQ ID No: 9, as follows:

[SEQ ID No: 9]
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAK

Therefore, preferably the second amino acid sequence (i.e. comprising the Ig-Fc region) comprises or consists of an amino acid sequence substantially as set out in SEQ ID No: 9, or a fragment or variant thereof.

In one embodiment, the nucleic acid sequence (which may be plant-codon optimised) encoding the CH₂ domain (human IgG1) is referred to herein as SEQ ID No: 10, as follows:

[SEQ ID No: 10]
GCTCCAGAACTTCTTGGCGGCCCATCCGTGTTTCTGTTCCCGCCGAAGCCAAAGGACACGCTGATGATCTCTCGCACC
CCAGAGGTGACATGCGTGGTGGTGGATGTGTCCCACGAGGACCCTGAGGTGAAGTTCAACTGGTATGTGGACGGCGTC
GAGGTGCACAACGCCAAGACAAAGCCGCGCGAGGAACAGTACAACTCCACCTACAGAGTGGTGTCCGTGCTCACCGTG
CTCCACCAGGATTGGCTGAACGGGAAAGAATACAAATGCAAGGTGTCCAACAAGGCGCTCCCAGCGCCGATCGAAAAG
ACCATCTCTAAGGCGAAG

Therefore, preferably the second amino acid sequence (i.e. comprising the Ig-Fc region) is encoded by a nucleotide sequence substantially as set out in SEQ ID No: 10, or a fragment or variant thereof.

Preferably, the CH₂ domain is disposed N-terminal of the CH₃ domain in the Ig-Fc region, or a fragment or variant thereof. Thus, preferably the Ig-Fc region, or a fragment or variant thereof comprises a CH₂—CH₃ domain of an immunoglobulin.

Thus, preferably the Ig-Fc region, or a fragment or variant thereof comprises SEQ ID No: 7 (mouse) and SEQ ID No: 3 (mouse), or SEQ ID No: 9 (human) and SEQ ID No: 5 (human).

Preferably, the fusion polypeptide of the invention comprises a fourth amino acid sequence comprising a hinge region of an immunoglobulin. Preferably, the hinge region is disposed N-terminal of the second amino acid comprising the IgG-Fc region, or a fragment or variant thereof. The hinge region may be selected from the hinge region of IgA, IgD, IgG, and/or the C domain of IgE and IgM. For each hinge region, all isotypes are considered. For example, for IgG, the isotypes IgG1, IgG2, IgG3, IgG4 can be used as the hinge region in the fusion polypeptide.

In a preferred embodiment, however, the hinge region is an IgG. Preferably, the IgG is a human IgG, more preferably a human IgG1. Preferably, the IgG is a mouse IgG, more preferably a mouse IgG2a.

Thus, the hinge region may be from a mouse, preferably IgG2a. In one embodiment, the amino acid sequence of the hinge region (mouse IgG2a) is referred to herein as SEQ ID No: 43, as follows:

[SEQ ID No: 43]
EPRGPTIKPSPPCKCP

Therefore, preferably the fourth amino acid sequence (i.e. comprising the hinge region) comprises or consists of an amino acid sequence substantially as set out in SEQ ID No: 43, or a fragment or variant thereof.

However, a point mutation can be introduced in the hinge region of mouse IgG2a at position Ile234Asn in the antibody heavy chain, or I→N in SEQ ID No: 43. Therefore, in a preferred embodiment, the hinge region of mouse IgG2a comprises a point mutation in SEQ ID No: 43 at position Ile234, which is most preferably Ile234Asn.

Thus, in one embodiment, the amino acid sequence of the hinge region (mouse IgG2a) comprising a point mutation is referred to herein as SEQ ID No: 11, as follows:

[SEQ ID No: 11]
EPRGPTNKPSPPCKCP

Therefore, preferably the fourth amino acid sequence (i.e. comprising the hinge region) comprises or consists of an amino acid sequence substantially as set out in SEQ ID No:11, or a fragment or variant thereof.

In one embodiment, the nucleic acid sequence (which may be plant-codon optimised) encoding the hinge region (mouse IgG2a) comprising a point mutation is referred to herein as SEQ ID No: 12, as follows:

[SEQ ID No: 12]
GAGCCAAGGGGCCCGACAAACAAGCCTTCTCCACCATGCAAGTGCCCA

Therefore, preferably the fourth amino acid sequence (i.e. comprising the hinge region) is encoded by a nucleotide sequence substantially as set out in SEQ ID No: 12, or a fragment or variant thereof.

The hinge region may be from a human, preferably IgG1. In one embodiment, the amino acid sequence of the hinge region (human IgG1) is referred to herein as SEQ ID No: 44, as follows:

[SEQ ID No: 44]
EPKSCDKTHTCPPCP

Therefore, preferably the fourth amino acid sequence (i.e. comprising the hinge region) comprises or consists of an amino acid sequence substantially as set out in SEQ ID No: 44, or a fragment or variant thereof.

However, a point mutation can be introduced in the hinge region of human IgG1 at position Cys230Ser in the antibody heavy chain, or C→S in SEQ ID No: 44, to ensure proper folding of the protein in the absence of the light chain. Therefore, in a preferred embodiment, the hinge region of human IgG1 comprises a point mutation in SEQ ID No: 44 at position Cys230, which is most preferably Cys230Ser.

Thus in one embodiment, the amino acid sequence of the hinge region (human IgG1) comprising a point mutation is referred to herein as SEQ ID No: 13, as follows:

[SEQ ID No: 13]
EPKSSDKTHTCPPCP

Therefore, preferably the fourth amino acid sequence (i.e. comprising the hinge region) comprises or consists of an amino acid sequence substantially as set out in SEQ ID No: 13, or a fragment or variant thereof.

In one embodiment, the nucleic acid sequence (which may be plant-codon optimised) encoding the hinge region (human IgG1) comprising a point mutation is referred to herein as SEQ ID No: 14, as follows:

[SEQ ID No: 14]
GAGCCGAAGTCCTCCGACAAGACCCATACTTGCCCACCGTGTCCA

Therefore, preferably the fourth amino acid sequence (i.e. comprising the hinge region) is encoded by a nucleotide sequence substantially as set out in SEQ ID No:14, or a fragment or variant thereof.

Preferably, the hinge region is disposed N-terminal of the CH₂ and/or CH₃ domain in the Ig-Fc region, or a fragment or variant thereof. Thus, preferably, the fusion polypeptide comprises a hinge-CH₂—CH₃ domain of an immunoglobulin.

Thus, preferably the fusion polypeptide thereof comprises SEQ ID No: 11 (mouse), SEQ ID No: 7 (mouse) and SEQ ID No: 3 (mouse), or SEQ ID No: 13 (human), SEQ ID No: 9 (human) and SEQ ID No: 5 (human).

Preferably, in some embodiments, the fusion polypeptide of the invention comprises a fifth amino acid sequence comprising a CH₁ domain of an immunoglobulin, or a truncation thereof. This CH₁ domain is required for retaining the immunoglobulin-like Y shape. Furthermore, advantageously, the CH₁ domain acts as a natural linker and, together with other linkers of the invention (i.e. general flexible (GP) or substantially flexible (EK) linkers), increases the structural flexibility of the CTB, thereby allowing its polymerisation. In other embodiments, however, the fifth amino acid sequence comprising a CH₁ domain is absent.

In one embodiment, the CH₁ domain is the full-length CH₁ domain of IgA, IgD, IgE, IgG and/or IgM. Preferably, however, the fusion polypeptide thereof comprises a truncated CH₁ domain of an immunoglobulin, wherein at least the last 5, 6, 7, 8, 9 or 10 amino acid residues from the C-terminus, which correspond to the final β-strand of the CH₁ domain of an immunoglobulin are absent, deleted or removed. Advantageously, removal of the terminal amino acids minimises flexible functionality of the fusion polypeptide. Preferably, at least the terminal 10 amino acids of the CH₁ domain are absent. A truncated CH₁ domain is denoted herein as ΔCH₁. Thus, preferably the fusion polypeptide (or the Ig-Fc region, or a fragment or variant thereof) comprises a ΔCH₁-hinge-CH₂—CH₃ domain of an immunoglobulin.

The CH₁ domain of an immunoglobulin, or a truncation thereof may be selected from IgA, IgD, IgE, IgG and/or IgM. For each CH₁ domain, or a truncation thereof, all isotypes are considered. For example, for IgG, the isotypes IgG1, IgG2, IgG3, IgG4 can be used as the CH₁ domain, or a truncation thereof in the fusion polypeptide.

In a preferred embodiment, however, the CH₁ domain, or a truncation thereof is an IgG. Preferably, the IgG is a human IgG, more preferably a human IgG1. Preferably, the IgG is a mouse IgG, more preferably a mouse IgG2a.

Thus, the CH₁ domain, or a truncation thereof may be from a mouse, preferably IgG2a. In one embodiment, the amino acid sequence of the CH₁ domain, or a truncation thereof (mouse IgG2a) is referred to herein as SEQ ID No: 15, as follows:

[SEQ ID No: 15]
ASSTKVDKKI

Therefore, preferably the fifth amino acid sequence (i.e. comprising the CH₁ domain, or a truncation thereof) comprises or consists of an amino acid sequence substantially as set out in SEQ ID No:15, or a fragment or variant thereof.

In one embodiment, the nucleic acid sequence (which may be plant codon optimised) encoding the CH₁ domain, or a truncation thereof (mouse IgG2a) is referred to herein as SEQ ID No: 16, as follows:

[SEQ ID No: 16]
GCCAGCTCTACCAAGGTGGACAAGAAGATC

Therefore, preferably the fifth amino acid sequence (i.e. comprising the CH₁ domain, or a truncation thereof) is encoded by a nucleotide sequence substantially as set out in SEQ ID No: 16, or a fragment or variant thereof.

The CH₁ domain, or a truncation thereof may be from a human, preferably IgG1. In one embodiment, the amino acid sequence of the CH₁ domain, or a truncation thereof (human IgG1) is referred to herein as SEQ ID No: 17, as follows:

[SEQ ID No: 17]
ASNTKVDKKV

Therefore, preferably the fifth amino acid sequence (i.e. comprising the CH₁ domain, or a truncation thereof) comprises or consists of an amino acid sequence substantially as set out in SEQ ID No: 17, or a fragment or variant thereof.

In one embodiment, the nucleic acid sequence (which may be plant-codon optimised) encoding the CH₁ domain, or a truncation thereof (human IgG1) is referred to herein as SEQ ID No: 18, as follows:

[SEQ ID No: 18]
GCCTCCAATACCAAGGTGGACAAGAAGGTC

Therefore, preferably the fourth amino acid sequence (i.e. comprising the CH₁ domain, or a truncation thereof) is encoded by a nucleotide sequence substantially as set out in SEQ ID No:18, or a fragment or variant thereof.

Preferably, the fusion polypeptide does not comprise an amino acid sequence comprising a light chain of an immunoglobulin, or a variant or fragment of a variable domain. Preferably, the fusion polypeptide does not comprise an amino acid sequence comprising a variable domain of an immunoglobulin, or a variant or fragment of a variable domain. For example, preferably the fusion protein does not comprise a heavy chain variable sequence (VH) and/or a light chain variable sequence (VL) of an antibody.

As shown in FIG. 1B, to further improve the structural and polymeric characteristics of the fusion polypeptide, the inventors have also, in some embodiments, inserted a tailpiece of an immunoglobulin (for example, a human IgM-tail piece) at or towards the C-terminal end of the Ig-Fc fragment of the fusion polypeptide. Advantageously, this additionally allows the fusion polypeptide to polymerise through the Fc component, in addition to CTB. As can be seen in FIG. 1B(s), this embodiment of the fusion polypeptide can polymerise to create a hexamer.

In a preferred embodiment, therefore, the fusion polypeptide of the invention comprises a sixth amino acid sequence comprising a tailpiece domain of an immunoglobulin, or a fragment thereof. This tailpiece domain or fragment thereof preferably facilitates polymerisation of the fusion polypeptide from both the Fc region or fragment or variant thereof and/or the CTB domain. In other embodiments, however, the sixth amino acid sequence comprising a tailpiece of an immunoglobulin is absent.

Preferably, the sixth amino acid sequence comprising a tailpiece domain of an immunoglobulin, or a fragment thereof is disposed at or towards the C-terminus of the fusion polypeptide. Preferably, the sixth amino acid sequence is disposed C-terminal of the IgG-Fc region, or a fragment of variant thereof. Preferably, the sixth amino acid sequence is disposed N-terminal of the retrieval signal peptide, preferably the ER retention signal peptide.

In one embodiment, the immunoglobulin tailpiece domain or fragment thereof is the tailpiece domain of IgA, IgD, IgE, IgG and/or IgM. Preferably, however, the fusion polypeptide thereof comprises a tailpiece domain or fragment thereof of an IgM immunoglobulin. IgM is traditionally represented as a single isoform—a “pentamer” composed of 10 μ-heavy chains, 10 light chains, and a single J chain linked by inter-μ chain disulfide bonds in a well-defined quaternary arrangement. However, IgM is also made as a hexamer that lacks the J chain. The J-chain stoichiometry is uncertain, and the disulfide bonds form in multiple arrangements.

In some embodiments, the IgM tailpiece domain or fragment thereof comprises at least 10, 12, 15 or all 18 amino acid residues of any of the 10 μ-heavy chains of IgM.

Preferably, the IgM tailpiece domain or fragment thereof (also known as “μ-tp”) is a human IgM tailpiece. In one embodiment, the amino acid sequence of the μ-tp, or a fragment thereof (human IgM) is referred to herein as SEQ ID No: 45, as follows:

[SEQ ID No: 45]
PTLYNVSLVMSDTAGTCY

Therefore, preferably the sixth amino acid sequence (i.e. comprising the human IgM tailpiece domain, or a fragment thereof) comprises or consists of an amino acid sequence substantially as set out in SEQ ID No:45, or a fragment or variant thereof.

In one embodiment, the nucleic acid sequence (which may be plant codon optimised) encoding the IgM tailpiece (human IgM) is referred to herein as SEQ ID No: 46, as follows:

[SEQ ID No: 46]
CCAACTCTCTACAACGTGTCCCTCGTGATGTCTGATACTGCTGGCACT
TGCTAC

Therefore, preferably the sixth amino acid sequence (i.e. comprising the human IgM tailpiece or a fragment thereof) is encoded by a nucleotide sequence substantially as set out in SEQ ID No: 46, or a fragment or variant thereof.

Preferably, the fusion polypeptide comprises an amino acid sequence comprising a light chain, or a variant or fragment of IgM. Preferably, the fusion polypeptide does not comprise an amino acid sequence comprising a light chain, or a variant or fragment of IgM.

Preferably, the third amino acid sequence comprising the antigen, or a fragment or variant thereof, is disposed between the first and second amino acid sequences, and most preferably it is disposed N-terminal of the fourth and fifth amino acid sequences.

In one embodiment, the antigen, or a fragment or variant thereof, may be derived or isolated from one or more different pathogens, which cause infection, in animals, and which may therefore require immunisation against the or each pathogen. The fusion protein is useful in treating, preventing or ameliorating systemic or mucosal infectious diseases in humans, or animals, including fish. Accordingly, the antigen component of the fusion protein can be isolated from different pathogens. In another embodiment, the antigen component of the fusion polypeptide is an antigen that is present in two or more pathogens, or a fusion antigen derived from two or more pathogens.

The antigen, or a fragment or variant thereof may be derived from any micro-organism. For example, the antigen, or a fragment or variant thereof may be derived or isolated from a bacterium, virus, fungus or protozoan.

The antigen, or a fragment or variant thereof may be a bacterial antigen. The bacterial antigen may derived from a bacterium selected from the group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus inkuenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae J Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp. Preferably, the bacterium is selected from the group consisting of Streptococcus pneumonia, Mycobacterium tuberculosis or Haemophilus Influenzae.

The antigen, or a fragment or variant thereof may be a viral antigen. The viral antigen may be derived from a virus selected from the group consisting of Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainfuenza virus types 1-4 (PIV), Mumps, Sendai viruses, Simian virus 5, Bovine parainfuenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS CoV-1, SARS-CoV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses. Preferably, the virus is selected from the group consisting of SARS CoV, SARS CoV2, MERS or Influenza.

The antigen, or a fragment or variant thereof may be a fungal antigen. The fungal antigen may be derived from a fungus selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp. Preferably, the fungi is selected from the group consisting of Aspergillus, Cryptococcus, or Pneumocystis.

The antigen, or a fragment or variant thereof may be a protozoan antigen. The protozoan antigen may be derived from a protozoan selected from the group consisting of: Entamoeba histolytica, Giardia lambli, Cryptosporidium parvum, Cyclospora cayatanensis and Toxoplasma.

In another embodiment, however, the antigen, or fragment or variant thereof, may be a tumour-associated antigen. In this embodiment, the fusion protein may be useful in cancer therapy, management or prevention, and in particular, mucosal cancers in humans or animals. Accordingly, the antigen component of the fusion protein may comprise the whole or part of a tumour cell, or whole or part of the tumour antigen. In an embodiment, the antigen component of the fusion polypeptide is a tumour-associated antigen that is expressed or present in one, two or more cancer types. In an embodiment, the fusion polypeptide can be used as a dual or broad range vaccine against two or more infectious diseases, such as Dengue-Zika; Classical swine fever-Porcine epidemic diarrhoea virus, etc.

In another embodiment, the fusion polypeptide can be used as a dual or broad range vaccine against one, two or more cancers.

The antigen, or a fragment or variant thereof may be a tumour-associated antigen. Typical tumour antigens, which may be used in the fusion polypeptide, include antigens from: breast cancer (e.g. HER-2 antigen); pancreatic cancer (e.g. Trop2, hMSLN, MUC1), prostate cancer (PSA, MUC2), bladder cancer (NY-ESO-1), colorectal cancer (CEA: carcinoembryonic antigen), leukaemia (WT 1), melanoma (MART-1, gp100, and tyrosinase), skin cancer (e.g. MAGE-3, MAA), lung cancer (e.g. CLDN18.2), non-small lung cell carcinoma (URLC10, VEGFR1 and VEGFR2), ovarian cancer (surviving, OV-TL3 and MOV18), renal tumour-associated antigen (e.g. G250, EGP-40), and cervical cancer (HPV16 E7: papillomaviridae E7). These antigens may be useful for vaccinating against any of these cancers using the fusion protein of the invention.

In one preferred embodiment, the third amino acid sequence comprises a Dengue antigen, or a fragment or variant thereof. In one embodiment, the amino acid sequence of the Dengue antigen is referred to herein as SEQ ID No: 19, as follows:

[SEQ ID No: 19]
KGMSYAMCTGKFKLEKEVAETQHGTILIKVKYEGDGAPCKIPFEIQDVE
KKHVNGRLITANPIVTDKESPVNIEAEPPFGDSYIVIGVGDKALKLNWF
KKGSS

Therefore, preferably the third amino acid sequence (i.e. comprising the antigen) comprises or consists of an amino acid sequence substantially as set out in SEQ ID No: 19, or a fragment or variant thereof.

In one embodiment, the nucleic acid sequence (which may be plant-codon optimised) encoding the Dengue antigen is referred to herein as SEQ ID No: 20, as follows:

[SEQ ID No: 20]
AAGGGCATGTCCTACGCTATGTGCACCGGCAAGTTCAAGTTGGAGAAGGAGGTGGCTGAGACCCAGCACGGCACCATC
TTGATCAAGGTGAAGTACGAGGGCGATGGCGCTCCTTGCAAGATCCCTTTCGAGATCCAGGATGTGGAGAAGAAGCAC
GTGAACGGAAGGTTGATCACCGCTAACCCTATCGTGACCGATAAGGAGTCCCCTGTGAACATCGAGGCTGAGCCTCCT
TTCGGCGATTCCTACATCGTGATCGGCGTGGGCGATAAGGCTTTGAAGTTGAACTGGTTCAAGAAGGGTTCTTCA

Therefore, preferably the third amino acid sequence (i.e. comprising the antigen) is encoded by a nucleotide sequence substantially as set out in SEQ ID No:20, or a fragment or variant thereof.

In another preferred embodiment, the third amino acid sequence comprises a SARS CoV-2 antigen, or a fragment or variant thereof. In one embodiment, the amino acid sequence of the SARS CoV-2 antigen is referred to herein as SEQ ID No: 21, as follows:

[SEQ ID No: 21]
QPTESIVRFPNITNLCPFGEVFNATRFASVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGNYNYLYRLFRKSNLKPFERDISTEIYQAG
STPCNGVEGENCYFPLQSYGFQPTNGVGYQPYRVVVLSFELLHAP

Therefore, preferably the third amino acid sequence (i.e. comprising the antigen) comprises or consists of an amino acid sequence substantially as set out in SEQ ID No: 20, or a fragment or variant thereof.

In one embodiment, the nucleic acid sequence (which may be plant-codon optimised) encoding the SARS CoV-2 antigen is referred to herein as SEQ ID No: 22, as follows:

[SEQ ID No: 22]
CAGCCAACCGAATCCATTGTGCGCTTCCCGAACATCACCAATCTGTGCCCATTCGGCGAGGTGTTCAACGCCACAAGA
TTCGCCTCTGTGTACGCCTGGAACCGCAAGCGCATCTCCAATTGCGTGGCCGACTACTCCGTGCTCTACAACTCTGCC
AGCTTCTCCACGTTCAAGTGCTACGGCGTGTCCCCGACCAAGCTGAACGATCTCTGCTTCACCAACGTGTACGCCGAC
TCCTTCGTGATCAGAGGCGACGAGGTGAGGCAAATTGCCCCAGGCCAGACAGGCAAGATCGCCGACTACAACTACAAG
CTCCCGGACGACTTCACCGGCTGCGTGATCGCTTGGAACTCCAACAACCTCGACTCCAAGGTCGGCGGCAACTACAAT
TACCTCTACCGCCTCTTCCGCAAGTCCAACCTCAAGCCGTTCGAGCGCGATATCTCCACCGAGATCTATCAGGCCGGA
AGCACCCCATGCAATGGCGTCGAAGGCTTCAACTGCTACTTCCCGCTCCAGTCCTACGGCTTCCAGCCTACAAATGGC
GTGGGCTACCAACCGTACAGGGTCGTCGTTCTCAGCTTCGAGCTGCTCCATGCTCCA

Therefore, preferably the third amino acid sequence (i.e. comprising the antigen) is encoded by a nucleotide sequence substantially as set out in SEQ ID No: 22, or a fragment or variant thereof.

In yet another preferred embodiment, the third amino acid sequence comprises a TB antigen, or a fragment or variant thereof. In one embodiment, the amino acid sequence of the TB is referred to herein as SEQ ID No: 23, as follows:

[SEQ ID No: 23]
MTEQQWNFAGIEAAASAIQGNVTSIHSLLDEGKQSLTKLAAAWGGSGSEA
YQGVQQKWDATATELNNALQNLARTISEAGQAMASTEGNVTGMFAGGGGG
MAEMKTDAATLAQEAGNFERISGDLKTQIDQVESTAGSLQGQWRGAAGTA
AQAAVVRFQEAANKQKQELDEISTNIRQAGVQYSRADEEQQQALSSQMGF

Therefore, preferably the third amino acid sequence (i.e. comprising the antigen) comprises or consists of an amino acid sequence substantially as set out in SEQ ID No: 23, or a fragment or variant thereof.

In one embodiment, the nucleic acid sequence (which may be plant-codon optimised) encoding the TB antigen is referred to herein as SEQ ID No: 24, as follows:

[SEQ ID No: 24]
ATGACTGAGCAGCAGTGGAATTTTGCTGGTATTGAGGCTGCTGCTTCTGC
TATTCAGGGTAATGTTACTTCTATTCATTCTCTTCTTGATGAGGGTAAAC
AGTCTCTTACTAAGTTGGCTGCTGCTTGGGGGGTTCTGGTTCTGAGGCTT
ACCAGGGTGTTCAGCAGAAGTGGGATGCTACTGCTACTGAGCTTAACAAT
GCTCTTCAGAATCTTGCTAGGACTATTTCTGAGGCTGGTCAGGCTATGGC
TTCTACTGAGGGTAATGTTACTGGTATGTTTGCTGGTGGTGGTGGTGGTA
TGGCTGAGATGAAGACTGATGCTGCTACTCTTGCTCAGGAGGCTGGTAAT
TTTGAGAGGATTTCTGGAGATTTGAAGACTCAGATTGATCAGGTTGAGTC
TACTGCTGGTTCTCTTCAGGGTCAGTGGAGGGGTGCTGCTGGTACTGCTG
CTCAGGCTGCTGTTGTTAGGTTTCAGGAGGCTGCTAATAAGCAGAAGCAG
GAGCTTGATGAGATTTCTACTAATATTAGGCAGGCTGGTGTTCAGTACTC
TAGGGCTGATGAGGAGCAGCAGCAGGCTCTTTCTTCTCAGATGGGTTTT

Therefore, preferably the third amino acid sequence (i.e. comprising the antigen) is encoded by a nucleotide sequence substantially as set out in SEQ ID No: 24, or a fragment or variant thereof.

The fusion polypeptide may comprise or be a fusion protein. The fusion polypeptide provided herein may be a single polypeptide monomer. Alternatively, the fusion polypeptide may polymerise, whereby a plurality of polypeptide monomers aggregate, combine or fuse together. For example, as shown in FIG. 1A or 1B (b), the single monomeric form(S) of the fusion polypeptide of the invention may dimerise to form a structure, which closely resembles the Y-shape of an IgG antibody. The dimerization is believed to occur via the Ig-Fc region, and in particular, the CH₂ domain and/or the CH₃ domain and/or the hinge region.

Furthermore, as shown in FIG. 1A or 1B (c), this Y-shaped dimer of the polypeptide of the invention, may polymerise, whereby a plurality of polypeptide Y-shaped dimers aggregate, combine or fuse together. As can be seen in FIG. 1c, polymerization can preferably create a monomer, dimer, trimer, tetramer, pentamer, hexamer or more, of the Y-shaped dimerised fusion polypeptide. Advantageously, this ability to polymerise exhibited by the fusion polypeptide allows the polypeptide(s) to bind to antigen presenting cells with higher affinity, thus increasing antigen uptake and presentation.

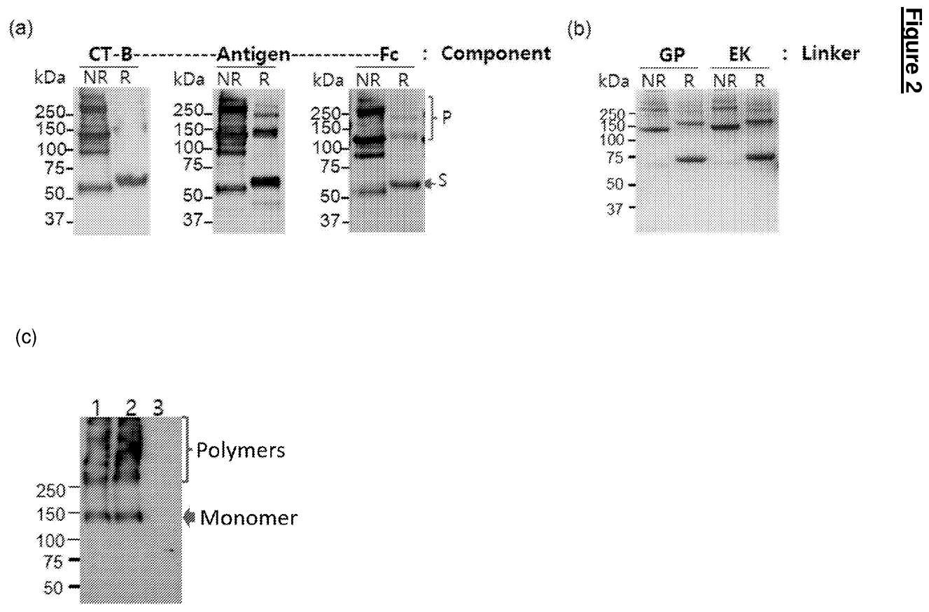

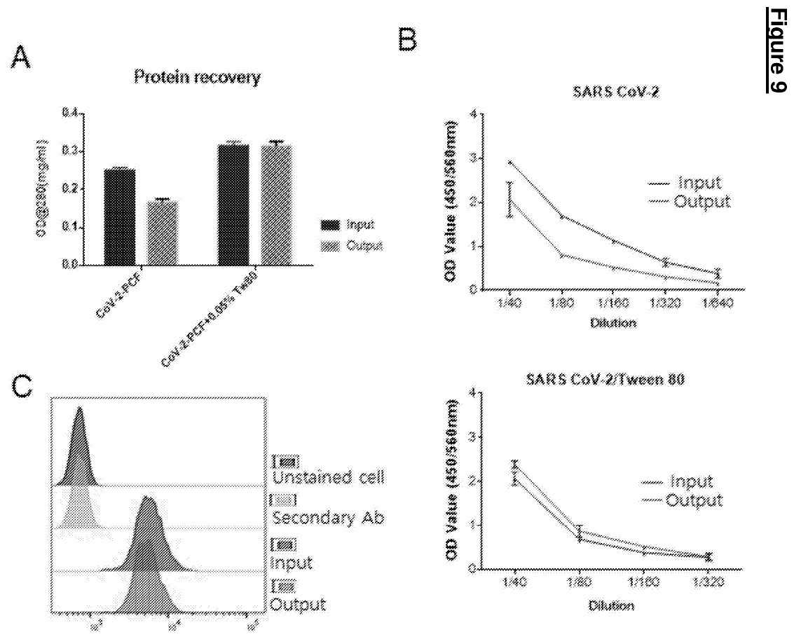

The polymerisation of the fusion polypeptide significantly improves its functionality and immunogenicity. Polymeric structures increase the quantity of the antigen delivered to antigen-presenting cells and, through the aggregation of the Fc molecules, enhance binding to high and low affinity receptors compared to monomeric Fc (as shown, for example, in FIG. 3c). Polymeric structures also facilitate better assembly of the antigen itself because, as described in the Examples, serum antibodies from Covid-19 vaccinated hosts bind more effectively to the polymeric RBD antigens of the SARS CoV2-PCF construct of the invention than to monomeric RBD antigens (see FIG. 10).

In a preferred embodiment, the fusion polypeptide comprises a signal peptide, which improves the level of expression and/or polymerisation of the fusion polypeptide in a host cell. Preferably, the signal peptide is disposed at or towards the N-terminus of the fusion polypeptide. The signal peptide may comprise any signal peptide or peptide from any organelle or pathway that can improve the expression and/or polymerization in situ, such as apoplasts, the ER or a secretion pathway.

Preferably, however, the signal peptide comprises an ER to Golgi trafficking signal peptide. Either the wild-type or a combined signal peptide of different origins may be used. For example, the signal peptide may comprise an ER to Golgi trafficking signal peptide of human IgG1 or of rice amylase 3D.

In one embodiment, the amino acid sequence of the ER to Golgi trafficking signal peptide (i.e. the human IgG1) is referred to herein as SEQ ID No: 25 as follows:

| [SEQ ID No: 25] | |
|---|---|
| | MELGLSWIFLLAILKGVQC |

Therefore, preferably signal peptide (i.e. the human IgG1) comprises or consists of an amino acid sequence substantially as set out in SEQ ID No: 25, or a fragment or variant thereof.

In one embodiment, the nucleic acid sequence (which may be plant-codon optimised) encoding the signal peptide of the human IgG1 is referred to herein as SEQ ID No: 26, as follows:

[SEQ ID No: 26]
ATGGAACTCGGCCTTTCTTGGATTTTCTTGCTCGCTATCCTCAAGGGCGT
GCAATGC

Therefore, preferably the signal peptide (i.e. the human IgG1) is encoded by a nucleotide sequence substantially as set out in SEQ ID No: 26, or a fragment or variant thereof.

In another embodiment, the amino acid sequence of the ER to Golgi trafficking signal peptide of the rice amylase 3D is referred to herein as SEQ ID No: 27, as follows:

| [SEQ ID No: 27] | |
|---|---|
| | MKNTSSLCLLLLVVLCSLTCNSGQA |

Therefore, preferably signal peptide (i.e. the rice amylase 3D) comprises or consists of an amino acid sequence substantially as set out in SEQ ID No: 27, or a fragment or variant thereof.

In one embodiment, the nucleic acid sequence (which may be plant-codon optimised) encoding the signal peptide of the rice amylase 3D is referred to herein as SEQ ID No: 28, as follows:

[SEQ ID No: 28]
ATGAAGAATACCTCCAGCCTCTGCCTCCTCCTCCTGGTGGTTCTCTGCTC
CCTCACATGCAATTCCGGCCAGGCC

Therefore, preferably the signal peptide (i.e. the rice amylase 3D) is encoded by a nucleotide sequence substantially as set out in SEQ ID No: 28, or a fragment or variant thereof.

In addition, or alternatively, any signal peptide and/or peptide from an organelle or pathway that can improve expression and polymerization in situ, such as apoplasts, ER or secretion pathway may be used as the retrieval signal peptide at the N-terminal region of the fusion protein. Thus, preferably the fusion polypeptide comprises a retrieval signal peptide, which improves the level of expression and/or polymerisation of the fusion polypeptide in a host cell. Preferably, the retrieval signal peptide is disposed at or towards the C-terminus of the fusion polypeptide.

Preferably, the retrieval signal peptide comprises an ER retention signal peptide. In an embodiment, the amino acid sequence of the ER retention signal peptide is referred to herein as SEQ ID No: 29, as follows:

| [SEQ ID No: 29] | |
|---|---|
| | SEKDEL |

Therefore, preferably the ER retention signal peptide comprises or consists of an amino acid sequence substantially as set out in SEQ ID No: 29, or a fragment or variant thereof.

In one embodiment, the nucleic acid sequence (which may be plant-codon optimised) encoding the ER retention signal peptide is referred to herein as SEQ ID No: 30, as follows:

| [SEQ ID No: 30] | |
|---|---|
| | TCCGAGAAGGATGAGCTT |

Therefore, preferably the retrieval signal peptide is encoded by a nucleotide sequence substantially as set out in SEQ ID No: 30, or a fragment or variant thereof.

In yet another preferred embodiment, the fusion polypeptide of the invention comprises one or more linker peptide, which may be disposed between first amino acid sequence, the second amino acid sequence and/or the third amino acid sequence (see FIG. 1a). The linker peptide is intended to facilitate polymerisation between the fusion polypeptide, as illustrated in FIG. 1c. Preferably, the polypeptide comprises a linker peptide disposed between the first amino acid sequence comprising the AB₅ toxin B subunit, or a fragment or a variant thereof, and the third amino acid sequence comprising the antigen, or a fragment of variant thereof.

Any flexible or rigid linker peptide of any sequence length (long or short) can be used in the present invention. In one embodiment, the linker peptide may be a substantially rigid linker peptide, such as a (GP) linker peptide. In an embodiment, the amino acid sequence of a GP linker peptide is referred to herein as SEQ ID No: 31, as follows:

| [SEQ ID No: 31] | |
|---|---|
| | GPGPGS |

Therefore, preferably the linker peptide comprises or consists of an amino acid sequence substantially as set out in SEQ ID No: 31, or a fragment or variant thereof.

In one embodiment, the nucleic acid sequence (which may be plant-codon optimised) encoding a GP linker peptide is referred to herein as SEQ ID No: 32, as follows:

| [SEQ ID No: 32] | |
|---|---|
| | GGCCCAGGCCCGGGATCC |

Therefore, preferably the linker peptide is encoded by a nucleotide sequence substantially as set out in SEQ ID No: 32, or a fragment or variant thereof.

Advantageously, a rigid linker, such as the (GP) linker, can be used as they are resistant to proteolytic degradation.

However, in other embodiments, a flexible linker may be preferred as they do not prevent polymerization, and so the polymeric structures shown in FIG. 1c can be readily created through bonding of the AB₅ toxin B subunit, such as CTB. Hence, in one embodiment, the linker peptide may be a substantially flexible linker peptide, such as a (EK) linker peptide. In an embodiment, the amino acid sequence of a EK linker peptide is referred to herein as SEQ ID No: 33, as follows:

| [SEQ ID No: 33] | |
|---|---|
| | AEAAAKEAAAKEAAAKA |

Therefore, preferably the linker peptide comprises or consists of an amino acid sequence substantially as set out in SEQ ID No: 33, or a fragment or variant thereof.

In one embodiment, the nucleic acid sequence (which may be plant-codon optimised) encoding a EK linker peptide is referred to herein as SEQ ID No: 34, as follows:

[SEQ ID No: 34]
GCCGAGGCCGCTGCTAAAGAGGCTGCCGCCAAAGAAGCTGCTGCCAAGGC
T

Therefore, preferably the linker peptide is encoded by a nucleotide sequence substantially as set out in SEQ ID No: 34, or a fragment or variant thereof.

In a preferred embodiment, the fusion polypeptide may comprise, in this specified order, (i) an “N-terminal” AB₅ toxin B subunit, or a fragment or variant thereof; (ii) an antigen, or a fragment or variant thereof; and (iii) a “C-terminal” immunoglobulin Fc region, or a fragment or variant thereof (Ig-Fc). The use of “N-terminal” and “C-terminal” indicates that a certain feature is either upstream or downstream with respect to another feature (i.e. relative positioning), and is not intended to indicate that the features are necessarily truly terminal features of the fusion polypeptide.

In a particular embodiment, the fusion polypeptide may comprise, in this specified order, (i) an “N-terminal” AB₅ toxin B subunit, or a fragment or variant thereof; (ii) an antigen, or a fragment or variant thereof; (iii) a CH₁ domain of an immunoglobulin, or a truncation thereof; and (iv) a “C-terminal” immunoglobulin Fc region, or a fragment or variant thereof (Ig-Fc).

In another particular embodiment, the fusion polypeptide may comprise, in this specified order, (i) an “N-terminal” AB₅ toxin B subunit, or a fragment or variant thereof; (ii) a linker peptide; (iii) an antigen, or a fragment or variant thereof; (iv) an optional CH₁ domain of an immunoglobulin, or a truncation thereof; and (v) a “C-terminal” immunoglobulin Fc region, or a fragment or variant thereof (Ig-Fc).

In yet another particular embodiment, the fusion polypeptide may comprise, in this specified order, (i) an “N-terminal” ER to Golgi trafficking signal peptide; (ii) an AB₅ toxin B subunit, or a fragment or variant thereof; (iii) a linker peptide; (iv) an antigen, or a fragment or variant thereof; (v) an optional CH₁ domain of an immunoglobulin, or a truncation thereof; and (vi) a “C-terminal” immunoglobulin Fc region, or a fragment or variant thereof (Ig-Fc).

In yet another particular embodiment, the fusion polypeptide may comprise, in this specified order, (i) an optional “N-terminal” ER to Golgi trafficking signal peptide; (ii) an AB₅ toxin B subunit, or a fragment or variant thereof; (iii) an optional linker peptide; (iv) an antigen, or a fragment or variant thereof; (v) an optional CH₁ domain of an immunoglobulin, or a truncation thereof; (vi) immunoglobulin Fc region, or a fragment or variant thereof (Ig-Fc); and (vii) an optional “C-terminal” ER retention signal peptide.

In yet another particular embodiment, the fusion polypeptide may comprise, in this specified order, (i) an optional “N-terminal” ER to Golgi trafficking signal peptide; (ii) an AB₅ toxin B subunit, or a fragment or variant thereof; (iii) an optional linker peptide; (iv) an antigen, or a fragment or variant thereof; (v) an optional CH₁ domain of an immunoglobulin, or a truncation thereof; (vi) immunoglobulin Fc region, or a fragment or variant thereof (Ig-Fc); (vii) an IgM tailpiece; and (viii) an optional “C-terminal” ER retention signal peptide.

As described in the examples, the inventors have created three embodiments (with and without the IgM tailpiece) of the fusion polypeptide of the invention, in which the antigen is either Dengue, SARS-CoV-2 or TB. The three embodiments lacking the IgM tailpiece will now be described.

Therefore, in a preferred embodiment in which the antigen is Dengue, the amino acid sequence of the fusion polypeptide based on human IgG1 with human IgG1 signal peptide is referred to herein as SEQ ID No: 35, as follows:

[SEQ ID No: 35]
MELGLSWIFLLAILKGVQCTPQNITDLCAEYHNTQIHTLNDKIFSYTESL
AGKREMAIITFKNGATFQVEVPGSQHIDSQKKAIERMKDTLRIAYLTEAK
VEKLCVWNNKTPHAIAAISMANGPGPGSKGMSYAMCTGKFKLEKEVAETQ
HGTILIKVKYEGDGAPCKIPFEIQDVEKKHVNGRLITANPIVTDKESPVN
IEAEPPFGDSYIVIGVGDKALKLNWFKKGSSTSASNTKVDKKVEPKSSDK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNV
FSCSVMHEALHNHYTQKSLSLSTGKSEKDEL

Therefore, preferably the fusion polypeptide comprises or consists of an amino acid sequence substantially as set out in SEQ ID No: 35, or a fragment or variant thereof.

Therefore, in a preferred embodiment in which the antigen is SARS-CoV-2, the amino acid sequence of the fusion polypeptide based on mouse IgG2a with rice amylase 3D signal peptide, is referred to herein as SEQ ID No: 36, as follows:

[SEQ ID No: 36]
MELGLSWIFLLAILKGVQCTPQNITDLCAEYHNTQIHTLNDKIFSYTESL
AGKREMAIITFKNGATFQVEVPGSQHIDSQKKAIERMKDTLRIAYLTEAK
VEKLCVWNNKTPHAIAAISMANGPGPGSQPTESIVRFPNITNLCPFGEVE
NATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLD
SKVGGNYNYLYRLFRKSNLKPFERDISTEIYQAGSTPCNGVEGENCYFPL
QSYGFQPTNGVGYQPYRVVVLSFELLHAPASSTKVDKKIEPRGPTNKPSP
PCKCPAPNLLGGPSVFIFPPKIKDVLMISLSPIVTCVVVDVSEDDPDVQI
SWFVNNVEVHTAQTQTHREDYNSTLRVVSALPIQHQDWMSGKEFKCKVNN
KDLPAPIERTISKPKGSVRAPQVYVLPPPEEEMTKKQVTLTCMVTDFMPE
DIYVEWTNNGKTELNYKNTEPVLDSDGSYFMYSKLRVEKKNWVERNSYSC
SVVHEGLHNHHTTKSFSRPTGKSEKDEL

Therefore, preferably the fusion polypeptide comprises or consists of an amino acid sequence substantially as set out in SEQ ID No: 36, or a fragment or variant thereof.

Therefore, in a preferred embodiment in which the antigen is SARS-CoV-2, the amino acid sequence of the fusion polypeptide based on human IgG1 with rice amylase 3D signal peptide is referred to herein as SEQ ID No: 37, as follows:

[SEQ ID No: 37]
MKNTSSLCLLLLVVLCSLTCNSGQATPQNITDLCAEYHNTQIHTLNDKIF
SYTESLAGKREMAIITFKNGATFQVEVPGSQHIDSQKKAIERMKDTLRIA
YLTEAKVEKLCVWNNKTPHAIAAISMANAEAAAKEAAAKEAAAKAQPTES
IVRFPNITNLCPFGEVENATRFASVYAWNRKRISNCVADYSVLYNSASFS
TFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQA
GSTPCNGVEGENCYFPLQSYGFQPTNGVGYQPYRVVVLSFELLHAPASNT
KVDKKVEPKSSDKTHTCPPCPAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPGKSEKDEL

Therefore, preferably the fusion polypeptide comprises or consists of an amino acid sequence substantially as set out in SEQ ID No: 37, or a fragment or variant thereof.

Therefore, in a preferred embodiment in which the antigen is TB, the amino acid sequence of the fusion polypeptide based on human IgG1 with human IgG1 signal peptide is referred to herein as SEQ ID No: 38, as follows:

[SEQ ID No: 38]
MELGLSWIFLLAILKGVQCTPQNITDLCAEYHNTQIHTLNDKIFSYTESLAGKREMAIITFKNGATFQVEVPGSQHID
SQKKAIERMKDTLRIAYLTEAKVEKLCVWNNKTPHAIAAISMANGPGPGSMTEQQWNFAGIEAAASAIQGNVTSIHSL
LDEGKQSLTKLAAAWGGSGSEAYQGVQQKWDATATELNNALQNLARTISEAGQAMASTEGNVTGMFAGGGGGMAEMKT
DAATLAQEAGNFERISGDLKTQIDQVESTAGSLQGQWRGAAGTAAQAAVVRFQEAANKQKQELDEISTNIRQAGVQYS
RADEEQQQALSSQMGFTSASNTKVDKKVEPKSSDKTHTCPPCPAPELLGGPSVFLFPPKPKDTLMISRTPEVTCVVVD
VSHEDPEVKFNWYVDGVEVHNAKTKPREEQYNSTYRVVSVLIVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSTGKSEKDEL

Therefore, preferably the fusion polypeptide comprises or consists of an amino acid sequence substantially as set out in SEQ ID No: 38, or a fragment or variant thereof.

It will be appreciated that the each of the three embodiments of the fusion polypeptide of the invention, in which the antigen is either Dengue, SARS-CoV-2 or TB, may further comprises a tailpiece of an immunoglobulin, or a fragment thereof. In a preferred embodiment, the tailpiece of an immunoglobulin is an IgM tailpiece, more preferably a human IgM tailpiece (μ-tp).

Therefore, in a preferred embodiment in which the antigen is Dengue, the amino acid sequence of the fusion polypeptide based on human IgG1 with human IgG1 signal peptide and comprising μ-tp and an ER-retention peptide (SEKDEL-SEQ ID No: 29) is referred to herein as SEQ ID No: 47, as follows:

[SEQ ID No: 47]
MELGLSWIFLLAILKGVQCTPQNITDLCAEYHNTQIHTLNDKIFSYTESLAGKREMAIITFKNGATFQVEVPGSQHID
SQKKAIERMKDTLRIAYLTEAKVEKLCVWNNKTPHAIAAISMANGPGPGSKGMSYAMCTGKFKLEKEVAETQHGTILI
KVKYEGDGAPCKIPFEIQDVEKKHVNGRLITANPIVTDKESPVNIEAEPPFGDSYIVIGVGDKALKLNWFKKGSSTSA
SNTKVDKKVEPKSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSVMHEALHNHYTQKS
LSLSTGKPTLYNVSLVMSDTAGTCYSEKDEL

Therefore, preferably the fusion polypeptide comprises or consists of an amino acid sequence substantially as set out in SEQ ID No: 47, or a fragment or variant thereof.

Therefore, in a preferred embodiment in which the antigen is SARS-CoV-2, the amino acid sequence of the fusion polypeptide based on mouse IgG2a with rice amylase 3D signal peptide, and comprising μ-tp is referred to herein as SEQ ID No: 48, as follows:

[SEQ ID No: 48]
MELGLSWIFLLAILKGVQCTPQNITDLCAEYHNTQIHTLNDKIFSYTESLAGKREMAIITFKNGATFQVEVPGSQHID
SQKKAIERMKDTLRIAYLTEAKVEKLCVWNNKTPHAIAAISMANGPGPGSQPTESIVRFPNITNLCPFGEVENATRFA
SVYAWNRKRISNCVADYSVLYNSASFSTFKCYGVSPTKLNDLCFTNVYADSFVIRGDEVRQIAPGQTGKIADYNYKLP
DDFTGCVIAWNSNNLDSKVGGNYNYLYRLFRKSNLKPFERDISTEIYQAGSTPCNGVEGENCYFPLQSYGFQPTNGVG
YQPYRVVVLSFELLHAPASSTKVDKKIEPRGPINKPSPPCKCPAPNLLGGPSVFIFPPKIKDVLMISLSPIVTCVVVD
VSEDDPDVQISWFVNNVEVHTAQTQTHREDYNSTLRVVSALPIQHQDWMSGKEFKCKVNNKDLPAPIERTISKPKGSV
RAPQVYVLPPPEEEMTKKQVTLTCMVTDFMPEDIYVEWTNNGKTELNYKNTEPVLDSDGSYFMYSKLRVEKKNWVERN
SYSCSVVHEGLHNHHTTKSFSRPTGKPTLYNVSLVMSDTAGTCYSEKDEL

Therefore, preferably the fusion polypeptide comprises or consists of an amino acid sequence substantially as set out in SEQ ID No: 48, or a fragment or variant thereof.

Therefore, in a preferred embodiment in which the antigen is SARS-CoV-2, the amino acid sequence of the fusion polypeptide based on human IgG1 with rice amylase 3D signal peptide and comprising μ-tp is referred to herein as SEQ ID No: 49, as follows

[SEQ ID No: 49]
MKNTSSLCLLLLVVLCSLTCNSGQATPQNITDLCAEYHNTQIHTLNDKIFSYTESLAGKREMAIITFKNGATFQVEVP
GSQHIDSQKKAIERMKDTLRIAYLTEAKVEKLCVWNNKTPHAIAAISMANAEAAAKEAAAKEAAAKAQPTESIVRFPN
ITNLCPFGEVENATRFASVYAWNRKRISNCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKSNLKPFERDISTEIYQAGSTPCNGVEGEN
CYFPLQSYGFQPTNGVGYQPYRVVVLSFELLHAPASNTKVDKKVEPKSSDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGKPTLYNVSLVMSDTAGTCYSEKDEL

Therefore, preferably the fusion polypeptide comprises or consists of an amino acid sequence substantially as set out in SEQ ID No: 49, or a fragment or variant thereof.

Therefore, in a preferred embodiment in which the antigen is TB, the amino acid sequence of the fusion polypeptide based on human IgG1 with human IgG1 signal peptide and comprising μ-tp is referred to herein as SEQ ID No: 50, as follows:

[SEQ ID No: 50]
MELGLSWIFLLAILKGVQCTPQNITDLCAEYHNTQIHTLNDKIFSYTESLAGKREMAIITFKNGATFQVEVPGSQHID
SQKKAIERMKDTLRIAYLTEAKVEKLCVWNNKTPHAIAAISMANGPGPGSMTEQQWNFAGIEAAASAIQGNVTSIHSL
LDEGKQSLTKLAAAWGGSGSEAYQGVQQKWDATATELNNALQNLARTISEAGQAMASTEGNVTGMFAGGGGGMAEMKT
DAATLAQEAGNFERISGDLKTQIDQVESTAGSLQGQWRGAAGTAAQAAVVRFQEAANKQKQELDEISTNIRQAGVQYS
RADEEQQQALSSQMGFTSASNTKVDKKVEPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSTGKPTLYNVSLVMSDTAGTCYSEKDEL

Therefore, preferably the fusion polypeptide comprises or consists of an amino acid sequence substantially as set out in SEQ ID No: 50, or a fragment or variant thereof.

It will be appreciated the fusion polypeptides of the invention may be encoded by a nucleic acid molecule.

Hence, in a second aspect of the invention, there is provided a nucleic acid encoding the fusion polypeptide according to the first aspect.

The nucleic acid may comprise DNA, RNA or a DNA/RNA hybrid sequence. Preferably, the nucleic acid comprises DNA or RNA. In one embodiment, the nucleic acid is a DNA sequence. The nucleic molecule may be used to express the fusion polypeptide.

As described herein, the inventors have created three embodiments of the fusion polypeptide of the invention, in which the antigen is either Dengue, SARS-Cov-2 or TB, and so they have created three embodiments of encoding DNA for these fusion polypeptides (lacking the IgM tailpiece).

Therefore, in a preferred embodiment in which the antigen is Dengue, the nucleic acid sequence of the fusion polypeptide (which may be plant-codon optimised) based on human IgG1 with human IgG1 signal peptide is referred to herein as SEQ ID No: 39, as follows:

[SEQ ID No: 39]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGGGATCCAAGGGC
ATGTCCTACGCTATGTGCACCGGCAAGTTCAAGTTGGAGAAGGAGGTGGCTGAGACCCAGCACGGCACCATCTTGATC
AAGGTGAAGTACGAGGGCGATGGCGCTCCTTGCAAGATCCCTTTCGAGATCCAGGATGTGGAGAAGAAGCACGTGAAC
GGAAGGTTGATCACCGCTAACCCTATCGTGACCGATAAGGAGTCCCCTGTGAACATCGAGGCTGAGCCTCCTTTCGGC
GATTCCTACATCGTGATCGGCGTGGGCGATAAGGCTTTGAAGTTGAACTGGTTCAAGAAGGGTTCATCTactagtGCT
TCCAACACAAAAGTTGATAAGAAGGTTGAGCCAAAGTCCTCCGATAAGACTCACACTTGTCCACCATGTCCAGCTCCA
GAACTTCTTGGAGGACCATCCGTTTTCTTGTTCCCACCAAAGCCAAAGGATACTCTCATGATCTCCAGGACTCCAGAG
GTTACATGCGTTGTGGTTGATGTGTCTCACGAGGATCCTGAGGTGAAGTTCAACTGGTATGTGGATGGTGTTGAGGTG
CACAACGCTAAGACTAAGCCACGTGAGGAACAGTACAACTCCACTTACAGGGTGGTGTCTGTGCTTACTGTGCTTCAC
CAGGATTGGCTTAACGGCAAAGAGTACAAGTGCAAGGTGTCCAACAAGGCTCTCCCAGCTCCAATCGAAAAGACTATC
TCCAAGGCTAAGGGCCAGCCAAGAGAGCCACAAGTTTACACTCTTCCACCATCCAGGGATGAGCTTACTAAGAACCAG
GTGTCCCTTACTTGCCTCGTGAAGGGATTCTACCCATCCGATATTGCTGTTGAGTGGGAGTCTAATGGCCAGCCTGAG
AACAACTACAAGACTACTCCACCAGTGCTCGATTCCGATGGCTCATTCTTCTTGTACTCCAAGCTCACTGTGGATAAG
TCCAGGTGGCAGCAGGGAAACGTTTTCTCTTGCTCTGTTATGCACGAGGCTCTCCACAACCACTACACTCAGAAGTCC
CTCTCACTCTCTACTGGCAAGTCCGAAAAGGATGAACTTTAA

Therefore, preferably the nucleic acid comprises or consists of a nucleotide sequence substantially as set out in SEQ ID No: 39, or a fragment or variant thereof.

In a preferred embodiment in which the antigen is SARS-Cov2, the nucleic acid sequence of the fusion polypeptide (which may be plant-codon optimised) based on mouse IgG2a based with rice amylase 3D signal peptide is referred to herein as SEQ ID No: 40, as follows:

[SEQ ID No: 40]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGGGATCCCAGCCA
ACCGAATCCATTGTGCGCTTCCCGAACATCACCAATCTGTGCCCATTCGGCGAGGTGTTCAACGCCACAAGATTCGCC
TCTGTGTACGCCTGGAACCGCAAGCGCATCTCCAATTGCGTGGCCGACTACTCCGTGCTCTACAACTCTGCCAGCTTC
TCCACGTTCAAGTGCTACGGCGTGTCCCCGACCAAGCTGAACGATCTCTGCTTCACCAACGTGTACGCCGACTCCTTC
GTGATCAGAGGCGACGAGGTGAGGCAAATTGCCCCAGGCCAGACAGGCAAGATCGCCGACTACAACTACAAGCTCCCG
GACGACTTCACCGGCTGCGTGATCGCTTGGAACTCCAACAACCTCGACTCCAAGGTCGGCGGCAACTACAATTACCTC
TACCGCCTCTTCCGCAAGTCCAACCTCAAGCCGTTCGAGCGCGATATCTCCACCGAGATCTATCAGGCCGGAAGCACC
CCATGCAATGGCGTCGAAGGCTTCAACTGCTACTTCCCGCTCCAGTCCTACGGCTTCCAGCCTACAAATGGCGTGGGC
TACCAACCGTACAGGGTCGTCGTTCTCAGCTTCGAGCTGCTCCATGCTCCAGCCAGCTCTACCAAGGTGGACAAGAAG
ATCGAGCCAAGGGGCCCGACAAACAAGCCTTCTCCACCATGCAAGTGCCCAGCGCCAAATCTTCTTGGCGGCCCATCC
GTGTTCATCTTCCCGCCGAAGATCAAGGACGTGCTCATGATCTCCCTCTCGCCGATCGTGACATGCGTGGTGGTGGAT
GTGTCCGAGGACGATCCGGATGTCCAGATCTCCTGGTTCGTGAACAACGTCGAGGTGCACACCGCGCAGACACAAACA
CACCGCGAGGACTACAATAGCACCCTCAGAGTGGTGAGCGCCCTGCCAATCCAGCACCAGGATTGGATGTCCGGGAAA
GAATTCAAGTGCAAGGTCAACAACAAGGACCTGCCAGCGCCGATCGAGAGGACCATCTCTAAGCCAAAGGGCTCCGTG
AGAGCCCCGCAAGTGTATGTTCTTCCGCCGCCAGAGGAAGAGATGACCAAGAAGCAAGTCACCCTGACCTGCATGGTG
ACCGACTTCATGCCAGAGGATATCTACGTCGAATGGACCAACAACGGCAAGACCGAGCTGAACTACAAGAACACCGAG
CCGGTGCTCGACTCCGACGGCTCCTACTTCATGTACTCCAAGCTCCGCGTCGAGAAGAAGAACTGGGTCGAGCGCAAC
TCCTACTCCTGCTCCGTTGTTCATGAGGGCCTCCACAACCACCACACCACCAAGTCTTTCTCCCGGCCAACTGGCAAG
TCCGAGAAGGATGAGCTTTGA

Therefore, preferably the nucleic acid comprises or consists of a nucleotide sequence substantially as set out in SEQ ID No: 40, or a fragment or variant thereof.

In a preferred embodiment in which the antigen is SARS-Cov2, the nucleic acid sequence of the fusion polypeptide (which may be plant-codon optimised) based on human IgG1 with rice amylase 3D signal peptide is referred to herein as SEQ ID No: 41, as follows:

[SEQ ID No: 41]
ATGAAGAATACCTCCAGCCTCTGCCTCCTCCTCCTGGTGGTTCTCTGCTCCCTCACATGCAATTCCGGCCAGGCCACA
CCACAGAACATCACAGATCTCTGCGCCGAGTACCACAACACCCAGATCCACACGCTCAACGACAAGATCTTCAGCTAC
ACCGAGAGCCTCGCTGGCAAGCGCGAGATGGCCATTATCACCTTCAAGAACGGCGCCACCTTCCAGGTTGAGGTGCCA
GGCTCTCAGCACATCGACTCCCAGAAGAAAGCCATCGAGCGCATGAAGGACACCCTCAGGATCGCCTACCTCACCGAG
GCCAAGGTTGAGAAGCTCTGCGTGTGGAACAACAAGACCCCGCATGCCATTGCCGCCATCTCTATGGCCAATGCCGAG
GCCGCTGCTAAAGAGGCTGCCGCCAAAGAAGCTGCTGCCAAGGCTCAGCCAACCGAATCCATTGTGCGCTTCCCGAAC
ATCACCAATCTGTGCCCATTCGGCGAGGTGTTCAACGCCACAAGATTCGCCTCTGTGTACGCCTGGAACCGCAAGCGC
ATCTCCAATTGCGTGGCCGACTACTCCGTGCTCTACAACTCTGCCAGCTTCTCCACGTTCAAGTGCTACGGCGTGTCC
CCGACCAAGCTGAACGATCTCTGCTTCACCAACGTGTACGCCGACTCCTTCGTGATCAGAGGCGACGAGGTGAGGCAA
ATTGCCCCAGGCCAGACAGGCAAGATCGCCGACTACAACTACAAGCTCCCGGACGACTTCACCGGCTGCGTGATCGCT
TGGAACTCCAACAACCTCGACTCCAAGGTCGGCGGCAACTACAATTACCTCTACCGCCTCTTCCGCAAGTCCAACCTC
AAGCCGTTCGAGCGCGATATCTCCACCGAGATCTATCAGGCCGGAAGCACCCCATGCAATGGCGTCGAAGGCTTCAAC
TGCTACTTCCCGCTCCAGTCCTACGGCTTCCAGCCTACAAATGGCGTGGGCTACCAACCGTACAGGGTCGTCGTTCTC
AGCTTCGAGCTGCTCCATGCGCCAGCCTCCAATACCAAGGTGGACAAGAAGGTCGAGCCGAAGTCCTCCGACAAGACC
CATACTTGCCCACCGTGTCCAGCTCCAGAACTTCTTGGCGGCCCATCCGTGTTTCTGTTCCCGCCGAAGCCAAAGGAC
ACGCTGATGATCTCTCGCACCCCAGAGGTGACATGCGTGGTGGTGGATGTGTCCCACGAGGACCCTGAGGTGAAGTTC
AACTGGTATGTGGACGGCGTCGAGGTGCACAACGCCAAGACAAAGCCGCGCGAGGAACAGTACAACTCCACCTACAGA
GTGGTGTCCGTGCTCACCGTGCTCCACCAGGATTGGCTGAACGGGAAAGAATACAAATGCAAGGTGTCCAACAAGGCG
CTCCCAGCGCCGATCGAAAAGACCATCTCTAAGGCGAAGGGCCAGCCAAGAGAGCCACAGGTTTACACACTTCCGCCG
TCCAGGGATGAGCTGACCAAGAACCAGGTGTCCCTGACCTGCCTCGTGAAGGGCTTCTACCCATCCGATATCGCCGTC
GAGTGGGAGTCCAATGGCCAGCCTGAGAACAATTACAAGACCACACCGCCGGTGCTCGACTCCGATGGCTCATTCTTC
CTGTACTCCAAGCTGACCGTCGACAAATCCAGATGGCAACAGGGCAACGTGTTCTCCTGCTCCGTTATGCACGAGGCC
CTCCACAACCACTACACCCAGAAGTCTCTCAGCCTCTCGCCAGGCAAGTCCGAGAAGGATGAGCTTTGA

Therefore, preferably the nucleic acid comprises or consists of a nucleotide sequence substantially as set out in SEQ ID No: 41, or a fragment or variant thereof.

In a preferred embodiment in which the antigen is TB, the nucleic acid sequence of the fusion polypeptide (which may be plant-codon optimised) based on human IgG1 with human IgG1 signal peptide is referred to herein as SEQ ID No: 42, as follows:

[SEQ ID No: 42]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGggatccATGACT
GAGCAGCAGTGGAATTTTGCTGGTATTGAGGCTGCTGCTTCTGCTATTCAGGGTAATGTTACTTCTATTCATTCTCTT
CTTGATGAGGGTAAACAGTCTCTTACTAAGTTGGCTGCTGCTTGGGGTGGTTCTGGTTCTGAGGCTTACCAGGGTGTT
CAGCAGAAGTGGGATGCTACTGCTACTGAGCTTAACAATGCTCTTCAGAATCTTGCTAGGACTATTTCTGAGGCTGGT
CAGGCTATGGCTTCTACTGAGGGTAATGTTACTGGTATGTTTGCTGGTGGTGGTGGTGGTATGGCTGAGATGAAGACT
GATGCTGCTACTCTTGCTCAGGAGGCTGGTAATTTTGAGAGGATTTCTGGAGATTTGAAGACTCAGATTGATCAGGTT
GAGTCTACTGCTGGTTCTCTTCAGGGTCAGTGGAGGGGTGCTGCTGGTACTGCTGCTCAGGCTGCTGTTGTTAGGTTT
CAGGAGGCTGCTAATAAGCAGAAGCAGGAGCTTGATGAGATTTCTACTAATATTAGGCAGGCTGGTGTTCAGTACTCT
AGGGCTGATGAGGAGCAGCAGCAGGCTCTTTCTTCTCAGATGGGTTTTactagtGCTTCCAACACAAAAGTTGATAAG
AAGGTTGAGCCAAAGTCCTCCGATAAGACTCACACTTGTCCACCATGTCCAGCTCCAGAACTTCTTGGAGGACCATCC
GTTTTCTTGTTCCCACCAAAGCCAAAGGATACTCTCATGATCTCCAGGACTCCAGAGGTTACATGCGTTGTGGTTGAT
GTGTCTCACGAGGATCCTGAGGTGAAGTTCAACTGGTATGTGGATGGTGTTGAGGTGCACAACGCTAAGACTAAGCCA
CGTGAGGAACAGTACAACTCCACTTACAGGGTGGTGTCTGTGCTTACTGTGCTTCACCAGGATTGGCTTAACGGCAAA
GAGTACAAGTGCAAGGTGTCCAACAAGGCTCTCCCAGCTCCAATCGAAAAGACTATCTCCAAGGCTAAGGGCCAGCCA
AGAGAGCCACAAGTTTACACTCTTCCACCATCCAGGGATGAGCTTACTAAGAACCAGGTGTCCCTTACTTGCCTCGTG
AAGGGATTCTACCCATCCGATATTGCTGTTGAGTGGGAGTCTAATGGCCAGCCTGAGAACAACTACAAGACTACTCCA
CCAGTGCTCGATTCCGATGGCTCATTCTTCTTGTACTCCAAGCTCACTGTGGATAAGTCCAGGTGGCAGCAGGGAAAC
GTTTTCTCTTGCTCTGTTATGCACGAGGCTCTCCACAACCACTACACTCAGAAGTCCCTCTCACTCTCTACTGGCAAG
TCCGAAAAGGATGAACTTTAA

Therefore, preferably the nucleic acid comprises or consists of a nucleotide sequence substantially as set out in SEQ ID No: 42, or a fragment or variant thereof.

As described herein, the inventors have created three embodiments of the fusion polypeptide of the invention further comprising a μ-tp, in which the antigen is either Dengue, SARS-Cov-2 or TB, and so they have also created embodiments of encoding DNA for these fusion polypeptides.

Therefore, in a preferred embodiment in which the antigen is Dengue, the nucleic acid sequence of the fusion polypeptide (which may be plant-codon optimised) based on human IgG1 with human IgG1 signal peptide and comprising μ-tp is referred to herein as SEQ ID No: 51, as follows:

[SEQ ID No: 51]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGGGATCCAAGGGC
ATGTCCTACGCTATGTGCACCGGCAAGTTCAAGTTGGAGAAGGAGGTGGCTGAGACCCAGCACGGCACCATCTTGATC
AAGGTGAAGTACGAGGGCGATGGCGCTCCTTGCAAGATCCCTTTCGAGATCCAGGATGTGGAGAAGAAGCACGTGAAC
GGAAGGTTGATCACCGCTAACCCTATCGTGACCGATAAGGAGTCCCCTGTGAACATCGAGGCTGAGCCTCCTTTCGGC
GATTCCTACATCGTGATCGGCGTGGGCGATAAGGCTTTGAAGTTGAACTGGTTCAAGAAGGGTTCATCTactagtGCT
TCCAACACAAAAGTTGATAAGAAGGTTGAGCCAAAGTCCTCCGATAAGACTCACACTTGTCCACCATGTCCAGCTCCA
GAACTTCTTGGAGGACCATCCGTTTTCTTGTTCCCACCAAAGCCAAAGGATACTCTCATGATCTCCAGGACTCCAGAG
GTTACATGCGTTGTGGTTGATGTGTCTCACGAGGATCCTGAGGTGAAGTTCAACTGGTATGTGGATGGTGTTGAGGTG
CACAACGCTAAGACTAAGCCACGTGAGGAACAGTACAACTCCACTTACAGGGTGGTGTCTGTGCTTACTGTGCTTCAC
CAGGATTGGCTTAACGGCAAAGAGTACAAGTGCAAGGTGTCCAACAAGGCTCTCCCAGCTCCAATCGAAAAGACTATC
TCCAAGGCTAAGGGCCAGCCAAGAGAGCCACAAGTTTACACTCTTCCACCATCCAGGGATGAGCTTACTAAGAACCAG
GTGTCCCTTACTTGCCTCGTGAAGGGATTCTACCCATCCGATATTGCTGTTGAGTGGGAGTCTAATGGCCAGCCTGAG
AACAACTACAAGACTACTCCACCAGTGCTCGATTCCGATGGCTCATTCTTCTTGTACTCCAAGCTCACTGTGGATAAG
TCCAGGTGGCAGCAGGGAAACGTTTTCTCTTGCTCTGTTATGCACGAGGCTCTCCACAACCACTACACTCAGAAGTCC
CTCTCACTCTCTACTGGCAAGCCAACTCTCTACAACGTGTCCCTCGTGATGTCTGATACTGCTGGCACTTGCTACTCCGA
AAAGGATGAACTTTAA

Therefore, preferably the nucleic acid comprises or consists of a nucleotide sequence substantially as set out in SEQ ID No: 51, or a fragment or variant thereof.

In a preferred embodiment in which the antigen is SARS-Cov2, the nucleic acid sequence of the fusion polypeptide (which may be plant-codon optimised) based on mouse IgG2a based with rice amylase 3D signal peptide and comprising μ-tp is referred to herein as SEQ ID No: 52, as follows:

[SEQ ID No: 52]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGGGATCCCAGCCA
ACCGAATCCATTGTGCGCTTCCCGAACATCACCAATCTGTGCCCATTCGGCGAGGTGTTCAACGCCACAAGATTCGCC
TCTGTGTACGCCTGGAACCGCAAGCGCATCTCCAATTGCGTGGCCGACTACTCCGTGCTCTACAACTCTGCCAGCTTC
TCCACGTTCAAGTGCTACGGCGTGTCCCCGACCAAGCTGAACGATCTCTGCTTCACCAACGTGTACGCCGACTCCTTC
GTGATCAGAGGCGACGAGGTGAGGCAAATTGCCCCAGGCCAGACAGGCAAGATCGCCGACTACAACTACAAGCTCCCG
GACGACTTCACCGGCTGCGTGATCGCTTGGAACTCCAACAACCTCGACTCCAAGGTCGGCGGCAACTACAATTACCTC
TACCGCCTCTTCCGCAAGTCCAACCTCAAGCCGTTCGAGCGCGATATCTCCACCGAGATCTATCAGGCCGGAAGCACC
CCATGCAATGGCGTCGAAGGCTTCAACTGCTACTTCCCGCTCCAGTCCTACGGCTTCCAGCCTACAAATGGCGTGGGC
TACCAACCGTACAGGGTCGTCGTTCTCAGCTTCGAGCTGCTCCATGCTCCAGCCAGCTCTACCAAGGTGGACAAGAAG
ATCGAGCCAAGGGGCCCGACAAACAAGCCTTCTCCACCATGCAAGTGCCCAGCGCCAAATCTTCTTGGCGGCCCATCC
GTGTTCATCTTCCCGCCGAAGATCAAGGACGTGCTCATGATCTCCCTCTCGCCGATCGTGACATGCGTGGTGGTGGAT
GTGTCCGAGGACGATCCGGATGTCCAGATCTCCTGGTTCGTGAACAACGTCGAGGTGCACACCGCGCAGACACAAACA
CACCGCGAGGACTACAATAGCACCCTCAGAGTGGTGAGCGCCCTGCCAATCCAGCACCAGGATTGGATGTCCGGGAAA
GAATTCAAGTGCAAGGTCAACAACAAGGACCTGCCAGCGCCGATCGAGAGGACCATCTCTAAGCCAAAGGGCTCCGTG
AGAGCCCCGCAAGTGTATGTTCTTCCGCCGCCAGAGGAAGAGATGACCAAGAAGCAAGTCACCCTGACCTGCATGGTG
ACCGACTTCATGCCAGAGGATATCTACGTCGAATGGACCAACAACGGCAAGACCGAGCTGAACTACAAGAACACCGAG
CCGGTGCTCGACTCCGACGGCTCCTACTTCATGTACTCCAAGCTCCGCGTCGAGAAGAAGAACTGGGTCGAGCGCAAC
TCCTACTCCTGCTCCGTTGTTCATGAGGGCCTCCACAACCACCACACCACCAAGTCTTTCTCCCGGCCAACTGGCAAG
CCAACTCTCTACAACGTGTCCCTCGTGATGTCTGATACTGCTGGCACTTGCTACTCCGAGAAGGATGAGCTTTGA

Therefore, preferably the nucleic acid comprises or consists of a nucleotide sequence substantially as set out in SEQ ID No: 52, or a fragment or variant thereof.

In a preferred embodiment in which the antigen is SARS-Cov2, the nucleic acid sequence of the fusion polypeptide (which may be plant-codon optimised) based on human IgG1 with rice amylase 3D signal peptide and comprising μ-tp is referred to herein as SEQ ID No: 53, as follows:

[SEQ ID No: 53]
ATGAAGAATACCTCCAGCCTCTGCCTCCTCCTCCTGGTGGTTCTCTGCTCCCTCACATGCAATTCCGGCCAGGCCACA
CCACAGAACATCACAGATCTCTGCGCCGAGTACCACAACACCCAGATCCACACGCTCAACGACAAGATCTTCAGCTAC
ACCGAGAGCCTCGCTGGCAAGCGCGAGATGGCCATTATCACCTTCAAGAACGGCGCCACCTTCCAGGTTGAGGTGCCA
GGCTCTCAGCACATCGACTCCCAGAAGAAAGCCATCGAGCGCATGAAGGACACCCTCAGGATCGCCTACCTCACCGAG
GCCAAGGTTGAGAAGCTCTGCGTGTGGAACAACAAGACCCCGCATGCCATTGCCGCCATCTCTATGGCCAATGCCGAG
GCCGCTGCTAAAGAGGCTGCCGCCAAAGAAGCTGCTGCCAAGGCTCAGCCAACCGAATCCATTGTGCGCTTCCCGAAC
ATCACCAATCTGTGCCCATTCGGCGAGGTGTTCAACGCCACAAGATTCGCCTCTGTGTACGCCTGGAACCGCAAGCGC
ATCTCCAATTGCGTGGCCGACTACTCCGTGCTCTACAACTCTGCCAGCTTCTCCACGTTCAAGTGCTACGGCGTGTCC
CCGACCAAGCTGAACGATCTCTGCTTCACCAACGTGTACGCCGACTCCTTCGTGATCAGAGGCGACGAGGTGAGGCAA
ATTGCCCCAGGCCAGACAGGCAAGATCGCCGACTACAACTACAAGCTCCCGGACGACTTCACCGGCTGCGTGATCGCT
TGGAACTCCAACAACCTCGACTCCAAGGTCGGCGGCAACTACAATTACCTCTACCGCCTCTTCCGCAAGTCCAACCTC
AAGCCGTTCGAGCGCGATATCTCCACCGAGATCTATCAGGCCGGAAGCACCCCATGCAATGGCGTCGAAGGCTTCAAC
TGCTACTTCCCGCTCCAGTCCTACGGCTTCCAGCCTACAAATGGCGTGGGCTACCAACCGTACAGGGTCGTCGTTCTC
AGCTTCGAGCTGCTCCATGCGCCAGCCTCCAATACCAAGGTGGACAAGAAGGTCGAGCCGAAGTCCTCCGACAAGACC
CATACTTGCCCACCGTGTCCAGCTCCAGAACTTCTTGGCGGCCCATCCGTGTTTCTGTTCCCGCCGAAGCCAAAGGAC
ACGCTGATGATCTCTCGCACCCCAGAGGTGACATGCGTGGTGGTGGATGTGTCCCACGAGGACCCTGAGGTGAAGTTC
AACTGGTATGTGGACGGCGTCGAGGTGCACAACGCCAAGACAAAGCCGCGCGAGGAACAGTACAACTCCACCTACAGA
GTGGTGTCCGTGCTCACCGTGCTCCACCAGGATTGGCTGAACGGGAAAGAATACAAATGCAAGGTGTCCAACAAGGCG
CTCCCAGCGCCGATCGAAAAGACCATCTCTAAGGCGAAGGGCCAGCCAAGAGAGCCACAGGTTTACACACTTCCGCCG
TCCAGGGATGAGCTGACCAAGAACCAGGTGTCCCTGACCTGCCTCGTGAAGGGCTTCTACCCATCCGATATCGCCGTC
GAGTGGGAGTCCAATGGCCAGCCTGAGAACAATTACAAGACCACACCGCCGGTGCTCGACTCCGATGGCTCATTCTTC
CTGTACTCCAAGCTGACCGTCGACAAATCCAGATGGCAACAGGGCAACGTGTTCTCCTGCTCCGTTATGCACGAGGCC
CTCCACAACCACTACACCCAGAAGTCTCTCAGCCTCTCGCCAGGCAAGCCAACTCTCTACAACGTGTCCCTCGTGATGT
CTGATACTGCTGGCACTTGCTACTCCGAGAAGGATGAGCTTTGA

Therefore, preferably the nucleic acid comprises or consists of a nucleotide sequence substantially as set out in SEQ ID No: 53, or a fragment or variant thereof.

In a preferred embodiment in which the antigen is TB, the nucleic acid sequence of the fusion polypeptide (which may be plant-codon optimised) based on human IgG1 with human IgG1 signal peptide and comprising μ-tp is referred to herein as SEQ ID No: 54, as follows:

[SEQ ID No: 54]
ATGGAACTCGGCCTTTCTTGGATTTTCTTGCTCGCTATCCTCAAGGGCGTGCAATGCACACCTCAAAATATTACTGAT
TTGTGTGCAGAATACCACAACACACAAATACATACGCTAAATGATAAGATATTTTCGTATACAGAATCTCTAGCTGGA
AAAAGAGAGATGGCTATCATTACTTTTAAGAATGGTGCGACTTTTCAAGTAGAAGTACCAGGTAGTCAACATATAGAT
TCACAAAAAAAAGCGATTGAAAGGATGAAGGATACCCTGAGGATTGCATATCTTACTGAAGCTAAAGTCGAAAAGTTA
TGTGTATGGAATAATAAAACGCCTCATGCGATTGCCGCAATTAGTATGGCAAATGGCCCAGGCCCGggatccATGACT
GAGCAGCAGTGGAATTTTGCTGGTATTGAGGCTGCTGCTTCTGCTATTCAGGGTAATGTTACTTCTATTCATTCTCTT
CTTGATGAGGGTAAACAGTCTCTTACTAAGTTGGCTGCTGCTTGGGGTGGTTCTGGTTCTGAGGCTTACCAGGGTGTT
CAGCAGAAGTGGGATGCTACTGCTACTGAGCTTAACAATGCTCTTCAGAATCTTGCTAGGACTATTTCTGAGGCTGGT
CAGGCTATGGCTTCTACTGAGGGTAATGTTACTGGTATGTTTGCTGGTGGTGGTGGTGGTATGGCTGAGATGAAGACT
GATGCTGCTACTCTTGCTCAGGAGGCTGGTAATTTTGAGAGGATTTCTGGAGATTTGAAGACTCAGATTGATCAGGTT
GAGTCTACTGCTGGTTCTCTTCAGGGTCAGTGGAGGGGTGCTGCTGGTACTGCTGCTCAGGCTGCTGTTGTTAGGTTT
CAGGAGGCTGCTAATAAGCAGAAGCAGGAGCTTGATGAGATTTCTACTAATATTAGGCAGGCTGGTGTTCAGTACTCT
AGGGCTGATGAGGAGCAGCAGCAGGCTCTTTCTTCTCAGATGGGTTTTactagtGCTTCCAACACAAAAGTTGATAAG
AAGGTTGAGCCAAAGTCCTCCGATAAGACTCACACTTGTCCACCATGTCCAGCTCCAGAACTTCTTGGAGGACCATCC
GTTTTCTTGTTCCCACCAAAGCCAAAGGATACTCTCATGATCTCCAGGACTCCAGAGGTTACATGCGTTGTGGTTGAT
GTGTCTCACGAGGATCCTGAGGTGAAGTTCAACTGGTATGTGGATGGTGTTGAGGTGCACAACGCTAAGACTAAGCCA
CGTGAGGAACAGTACAACTCCACTTACAGGGTGGTGTCTGTGCTTACTGTGCTTCACCAGGATTGGCTTAACGGCAAA
GAGTACAAGTGCAAGGTGTCCAACAAGGCTCTCCCAGCTCCAATCGAAAAGACTATCTCCAAGGCTAAGGGCCAGCCA
AGAGAGCCACAAGTTTACACTCTTCCACCATCCAGGGATGAGCTTACTAAGAACCAGGTGTCCCTTACTTGCCTCGTG
AAGGGATTCTACCCATCCGATATTGCTGTTGAGTGGGAGTCTAATGGCCAGCCTGAGAACAACTACAAGACTACTCCA
CCAGTGCTCGATTCCGATGGCTCATTCTTCTTGTACTCCAAGCTCACTGTGGATAAGTCCAGGTGGCAGCAGGGAAAC
GTTTTCTCTTGCTCTGTTATGCACGAGGCTCTCCACAACCACTACACTCAGAAGTCCCTCTCACTCTCTACTGGCAAG
CCAACTCTCTACAACGTGTCCCTCGTGATGTCTGATACTGCTGGCACTTGCTATCCGAAAAGGATGAACTTTAA

Therefore, preferably the nucleic acid comprises or consists of a nucleotide sequence substantially as set out in SEQ ID No: 54, or a fragment or variant thereof.

In a third aspect, there is provided an expression cassette comprising the nucleic acid according to the second aspect.

Preferably, the cassette comprises a promoter and/or an enhancer operably linked to the nucleic acid sequence encoding the fusion polypeptide.

The promoter may be a constitutive, inducible, tissue-specific or a synthetic promoter.

In one embodiment, the constitute promoter may be the 35S/duplicate 35S promoter.

In another embodiment, the inducible promoter may be the Ramy 3D promoter.

In yet another embodiment, the tissue-specific promoter maybe selected from PsDREB2 and 8SGα promoters.

In one embodiment, synthetic promoters with enhanced cell-state specificity (SPECS) can be used with 5′ untranslated region (or UTR) at an upstream of nucleic acid sequence encoding the fusion polypeptide.

In a fourth aspect of the invention, there is provided a recombinant vector comprising the nucleic acid according to the second aspect or the expression cassette of the third aspect.

The vector of the fourth aspect encoding the fusion protein of the first aspect may for example be a plasmid, cosmid or phage and/or be a viral vector. Such recombinant vectors are highly useful in the delivery systems of the invention for transforming cells with the nucleotide sequences of the second aspect. The nucleotide sequences may preferably be a DNA sequence.

Recombinant vectors encoding the RNA construct of the first aspect may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell, such as a bacterial cell, or a plant cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, Rice amylase 3D, EF1a, PGK, viral long terminal repeats, as well as inducible promoters, such as the Tetracycline inducible system, as examples. The cassette or vector may also comprise a terminator, such as the CMV, Rice amylase 3D, Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene(s). The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of the host cell. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated. When this is the case, regulation of expression may be less important because expression of the DNA molecule will stop when the transformed cells die or stop expressing the protein.

Alternatively, the delivery system may provide the nucleic acid molecule to the host cell without it being incorporated in a vector. For instance, the nucleic acid molecule may be incorporated within a liposome or virus particle. Alternatively a “naked” nucleic acid molecule may be inserted into a host cell by a suitable means, e.g. direct endocytotic uptake.

The success of plant expression technology has been demonstrated recently by the promising clinical trial results obtained by the Medicago/GSK SARS-Cov2 vaccine expressed in plants (Ward, Nat. Med. 2021). However, this Virus Like Particle (VLP)-based vaccine candidate still needs a powerful adjuvant (ASO3) from GSK, which may have potential safety concerns in some target populations.

Similarly to other licensed vaccines and current vaccine candidates which induce minimal mucosal immune responses, but are not specifically designed to induce a robust immune response in the mucosae, it is not clear whether the plant-based Medicago/GSK SARS-Cov2 vaccine would be applicable through the mucosal route (i.e. by inhalation) given the requirement of such a strong adjuvant. Conversely, the fusion polypeptide of the invention specifically targets the mucosae.

Advantageously, the inventors have surprisingly demonstrated in in vivo studies the ability of the fusion polypeptide of the invention to induce strong systemic and mucosal immune responses without an exogenous adjuvant at a relatively low dosage (i.e. at 10 μg of antigen equivalent or lower). Thus, the fusion polypeptide of the invention displays enhanced bioactivity and immunogenicity due to its self-adjuvanting attributes and superior structural and polymeric properties.

However, immunogenicity can be even further enhanced by simultaneously using an exogenous adjuvant. The adjuvant may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Saponins (Quil-A, QS-21, Tomatine, ISCOM, ISCOMATRIX™), squalene based emulsions, polymers such as PEI, Carbopol, lipid nanoparticles and bacterial toxins (CT, LT). As described in the Examples, the adjuvant Quil-A further enhanced the systemic antibody response triggered by the fusion polypeptide of the invention, and so is preferably administered with the fusion polypeptide.

The polypeptide of the invention was uniquely produced in plants by transient expression, offering lower production costs compared to mammalian cell expression system, easy scalability with cell suspension culture or transformed seeds and minimal safety concerns regarding human/animal pathogens associated with mammalian cell production.

However, the fusion polypeptide of the present invention can be expressed in one of the more conventional expression systems, such as mammalian cells. However, the expressing vaccine candidates and therapeutics, such as antibodies, in plants is preferred, and shows considerable promise.

Success examples of this technology is illustrated by the deployment of the ZMapp antibody during the 2014 Ebola epidemic (Lyon et al., 2014) and the Phase III clinical trial of a quadrivalent seasonal influenza VLP vaccine by Medicago Inc. (https://clinicaltrials.gov/ct2/show/NCT03739112) (Pillet et al., 2019), as well as the afore mentioned Medicago/GSK SARS-Cov2 vaccine (Ward, Nat. Med. 2021).

In a fifth aspect of the invention, there is provided a host cell comprising the recombinant vector of the fourth aspect.

The host cell may be a bacterial, yeast, viral, fungal, plant, mammalian or insect cell.

As described herein, the inventors have used plant codon optimised nucleic acid sequences in order to express the fusion polypeptide of the first aspect in plants. Preferably, therefore, the host cell is a plant cell. Any plant cell, such as callus, whole plant, or seed can be used to express the fusion polypeptide of the first aspect, using the vector of the fourth aspect.

Preferably, the production system using the host cell is a transformed cell suspension culture and a transient expression system and/or transgenic plants, via bioreactor and plant factory, respectively.

In a sixth aspect of the invention, there is provided a method for producing the fusion polypeptide of the first aspect, the method comprising the steps of:
(a) (i) introducing, into a host cell, the recombinant vector of the fourth aspect; and
   (ii) culturing the host cell under conditions to result in the production of the fusion polypeptide of the first aspect; or
(b) translating the polypeptide from the vector according to the fourth aspect.

The host cell of step (a) may be a eukaryotic or prokaryotic host cell. The host cell may be a plant cell. Suitable prokaryotic cells are bacterial cell, such as E. coli. Preferably, the host cell is a eukaryotic host cell. The host cell may be a yeast cell. The host cell may be a mammalian host cell such as Human embryonic kidney 293 cells or Chinese hamster ovary (CHO) cells. Step (b) may be performed in vitro or in vivo, preferably in vitro.

Preferably, the recombinant vector of the fourth aspect comprises a nucleic acid sequence encoding the fusion polypeptide of the first aspect. Preferably, the vector comprises a backbone nucleic acid sequence for the AB₅ toxin B subunit, or a fragment or variant thereof (preferably, CTB); the antigen, or a fragment or variant thereof; and Ig-Fc.

Preferably, the method comprises transforming a plant cell with the recombinant vector and storing plant seeds or plant cell transient expression of the nucleotide sequence of the vector.

Preferably, the method comprises expressing the fusion polypeptide from the vector nucleotide sequence.

Preferably, the method comprises detecting and/or measuring the immunogenicity of the fusion polypeptide.

In a seventh aspect, there is provided a fusion polypeptide obtained, or obtainable, by the method of the sixth aspect.

The fusion polypeptide of the first or seventh aspect is particularly suitable for producing a pharmaceutical composition.

Thus, in an eighth aspect, there is provided a pharmaceutical composition comprising the fusion polypeptide of the first or seventh aspect, the nucleic acid sequence of the second aspect, the expression cassette of the third aspect or the recombinant vector of the fourth aspect, and a pharmaceutically acceptable vehicle.

In a ninth aspect, there is provided a process for making the pharmaceutical composition according to the eighth aspect, the method comprising contacting the fusion polypeptide of the first or seventh aspect, the nucleic acid sequence of the second aspect, the expression cassette of the third aspect or the recombinant vector of the fourth aspect, with a pharmaceutically acceptable vehicle.

The fusion polypeptide is useful in therapy and prophylaxis, e.g. in a vaccine, or for treating or preventing cancer, depending on which antigen it encodes.

According to a tenth aspect, there is provided the fusion polypeptide according to the first or seventh aspect, the nucleic acid according to the second aspect, the expression cassette according to the third aspect, the recombinant vector according to the fourth aspect or the pharmaceutical composition according to the eighth aspect, for use as a medicament, or in therapy or prophylaxis.

Accordingly, in an eleventh aspect of the invention, there is provided a vaccine comprising the fusion polypeptide according to the first or seventh aspect, the nucleic acid according to the second aspect, the expression cassette according to the third aspect, the recombinant vector according to the fourth aspect or the pharmaceutical composition according to the eighth aspect.

In some embodiments, the vaccine may not comprise an exogenous adjuvant. This is because of the self-adjuvanting nature of CTB.

The vaccine may also be formulated with conventional adjuvants or delivery systems for enhanced immunogenicity.

For example, this may be the situation in which the polypeptide is formulated in a lipid-based nanoparticle or Lipid Nano Particle (LNP).

However, in other embodiments, the vaccine may comprise an exogenous adjuvant. The adjuvant may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Saponins (Quil-A, QS-21, Tomatine, ISCOM, ISCOMATRIX™), squalene based emulsions, polymers such as PEI, Carbopol, lipid nanoparticles and bacterial toxins (CT, LT). As described in the Examples, the adjuvant Quil-A further enhanced the systemic antibody response triggered by the fusion polypeptide of the invention, and so is preferably administered in the vaccine.

The fusion polypeptide described herein provides an effective means of vaccinating a subject against an infection, or for treating/preventing cancer.

Thus, in a twelfth aspect, there is provided the fusion polypeptide according to the first or seventh aspect, the nucleic acid according to the second aspect, the expression cassette according to the third aspect, the recombinant vector according to the fourth aspect, the pharmaceutical composition according to the eighth aspect, or the vaccine according to the eleventh aspect, for use in treating, preventing or ameliorating an infection or cancer.

In a thirteenth aspect, there is provided a method of treating, preventing or ameliorating an infection or cancer, the method comprising administering, or having administered, to a subject in need thereof, a therapeutically effective amount of the fusion polypeptide according to the first or seventh aspect, the nucleic acid according to the second aspect, the expression cassette according to the third aspect, the recombinant vector according to the fourth aspect, the pharmaceutical composition according to the eighth aspect, or the vaccine according to the eleventh aspect.

The infection may be caused by a micro-organism, such as a bacterium, virus, fungus or protozoan.

Preferably, the vaccine is mucosally administrable, preferably intranasally administrable.

In a fourteenth aspect of the invention, there is provided the fusion polypeptide according to the first or seventh aspect, the nucleic acid according to the second aspect, the expression cassette according to the third aspect, the recombinant vector according to the fourth aspect, the pharmaceutical composition according to the eighth aspect, or the vaccine of the eleventh aspect, for use in stimulating an immune response in a subject.

It will be appreciated that the fusion polypeptide according to the first or seventh aspect, the nucleic acid according to the second aspect, the expression cassette according to the third aspect, the recombinant vector according to the fourth aspect, the pharmaceutical composition according to the eighth aspect, or the vaccine of the eleventh aspect (herein known as the active agents) may be used in a medicament, which may be used as a monotherapy (i.e. use of the active agent), for vaccination against an infection. Alternatively, the active agents according to the invention may be used as an adjunct to, or in combination with, known therapies for treating, ameliorating, or preventing infections.

The fusion polypeptide according to the first or seventh aspect, the nucleic acid according to the second aspect, the expression cassette according to the third aspect, the recombinant vector according to the fourth aspect, the pharmaceutical composition according to the eighth aspect, or the vaccine of the eleventh aspect may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles or any other suitable form that may be administered to a person or animal in need of vaccination. It will be appreciated that the vehicle of medicaments according to the invention should be one which is well-tolerated by the subject to whom it is given.

The fusion polypeptide, the nucleic acid, expression cassette, recombinant vector, pharmaceutical composition, or vaccine of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the active agent is required and which would normally require frequent administration (e.g. at least daily injection).

In one embodiment, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion), or intramuscular (bolus or infusion). Preferably, the medicaments may be administered systemically (i.e. injection).

More preferably, however, the medicaments may be administered mucosally, which may be orally or by inhalation. Inhalation may comprise either nasal or oral administration.

It will be appreciated that the amount of fusion polypeptide, the nucleic acid, expression cassette, recombinant vector, pharmaceutical composition, or vaccine of the invention that is required is determined by its biological activity and bioavailability, which in turn depends on the mode of administration, the physiochemical properties of the fusion polypeptide, the nucleic acid, expression cassette, recombinant vector, pharmaceutical composition, or vaccine of the invention and whether it is being used as a monotherapy or in a combined therapy. The frequency of administration will also be influenced by the half-life of the active agent within the subject being treated. Optimal dosages to be administered may be determined by those skilled in the art, and will vary with the particular the fusion polypeptide, the nucleic acid, expression cassette, recombinant vector, pharmaceutical composition, or vaccine of the invention in use, the strength of the pharmaceutical composition, the mode of administration, and the type and advancement of the infection. Additional factors depending on the particular subject being treated will result in a need to adjust dosages, including subject age, weight, gender, diet, and time of administration.

Generally, a daily dose of between 0.001 μg/kg of body weight and 1 mg/kg of body weight, or between 0.01 μg/kg of body weight and 0.1 mg/kg of body weight, of the fusion polypeptide, the nucleic acid, expression cassette, recombinant vector, pharmaceutical composition, or vaccine of the invention may be used for the immunisation, depending upon the active agent being used.

Daily doses may be given as a single administration (e.g. a single daily injection or inhalation of a nasal spray). Alternatively, the fusion polypeptide, the nucleic acid, expression cassette, recombinant vector, pharmaceutical composition, or vaccine of the invention may require administration twice or more times during a day. As described in the examples, the fusion polypeptide, the nucleic acid, expression cassette, recombinant vector, pharmaceutical composition, or vaccine of the invention may be administered as an initial primer, and a subsequent boost, or two boosts administered at between a week or monthly intervals. Thus, a prime and boost regime is preferred. In a typical example, the active agent may be administered between 0 and 4 weeks apart.

As described in the Examples, the boost may be intranasally or mucosally administered. The inventors believes that the data described herein support the conclusion that the mucosal route of administration is the most effective and therefore preferred in inducing local cellular and humoral immunity in the lungs.

Known procedures, such as those conventionally employed by the pharmaceutical industry (e.g. in vivo experimentation, clinical trials, etc.), may be used to form specific formulations of the fusion polypeptide, the nucleic acid, expression cassette, recombinant vector, pharmaceutical composition, or vaccine of the invention according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications, such as fish. Most preferably, however, the subject is a human being.

A “therapeutically effective amount” of the fusion polypeptide, the nucleic acid, expression cassette, recombinant vector, pharmaceutical composition, or vaccine of the invention is any amount which, when administered to a subject, is the amount of the aforementioned that is needed to ameliorate, prevent or treat any given disease, preferably prophylactically.

For example, the fusion polypeptide, the nucleic acid, expression cassette, recombinant vector, pharmaceutical composition, or vaccine of the invention of the invention may be used may be from about 0.001 μg to about 1 mg, and preferably from about 0.001 μg to about 500 μg. It is preferred that the amount of the fusion polypeptide, the nucleic acid, expression cassette, recombinant vector, pharmaceutical composition, or vaccine of the invention is an amount from about 0.01 μg to about 250 μg, and most preferably from about 0.1 μg to about 100 μg. Preferably, the fusion polypeptide, the nucleic acid, expression cassette, recombinant vector, pharmaceutical composition, or vaccine of the invention according to the invention is administered at a dose of 1-50 μg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. fusion polypeptide, the nucleic acid, expression cassette, recombinant vector, pharmaceutical composition, or vaccine of the invention according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The fusion polypeptide, the nucleic acid, expression cassette, recombinant vector, pharmaceutical composition, or vaccine of the invention according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The fusion polypeptide, the nucleic acid, expression cassette, recombinant vector, pharmaceutical composition, or vaccine of the invention may be prepared as a sterile solid composition that may be dissolved or suspended at the time of administration using sterile water, saline, or other appropriate sterile injectable medium.

The fusion polypeptide, the nucleic acid, expression cassette, recombinant vector, pharmaceutical composition, or vaccine of the invention of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The fusion polypeptide, the nucleic acid, expression cassette, recombinant vector, pharmaceutical composition, or vaccine of the invention according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Experimental evidence has shown that polymeric Fc-fusion polypeptide of the invention is highly immunogenic against dengue, TB and coronavirus. The addition of the CTB component resulted in surprisingly superior, long-lasting IgA responses in Broncho-alveolar lavage in mice, against dengue. This novel, protein-based vaccine platform offers a further advantage of ease of purification using conventional Protein A/G affinity chromatography, and is perfectly suited for human application of purified protein via either the mucosal (nasal or oral) or systemic (injection) route.

The skilled person will appreciate that the present invention, when coupled with plant-based biotechnology as discussed above, presents at least three unique advantages:
(i) The possibility to administer the vaccine in the form of a single polymeric fusion protein without exogenous adjuvants;
(ii) The ability to produce it in plants with a potential for large scale production at a lower cost compared to other production systems; and
(iii) The potential for the respiratory route delivery.

In another preferred embodiment, the present invention is not restricted to the delivery of the purified fusion protein only, and can be extended to oral delivery of Ag—PCF within whole cells, such as transformed plants or yeasts, as edible crude vaccine for veterinary purposes.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequences identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:—

FIG. 1A provides a schematic representation of one embodiment of a fusion polypeptide according to the invention, for use as a vaccine delivery system. (a) represents a linear sequence of the fusion polypeptide. As shown, the polypeptide include an N-terminal cholera toxin B (CT-B), a C-terminal IgG Fc fragment (truncated ΔcH), and an internal Antigen. This mucosal/systemic vaccine delivery platform is referred to here as “Platform CTB-Fc”, or “PCF”; (b) shows a homo-monomeric structure of one embodiment of a vaccine delivery scaffold of the invention (right) compared to a conventional IgG heavy chain (left); (c) is a schematic representation of the fusion polypeptide pentamerisation through the CT-B sequences on each polypeptide monomer.

FIG. 1B provides a schematic representation of a second embodiment of a fusion polypeptide according to the invention, for use as a vaccine delivery system. (a) represents a linear sequence of the fusion polypeptide. As shown, the polypeptide includes an N-terminal cholera toxin B (CT-B), a C-terminal IgG Fc fragment (truncated ΔcH) followed by an IgM tail piece (m-tp), and an internal antigen. This mucosal/systemic vaccine delivery platform is also referred to as “Platform CTB-Fc”, or “PCF”; (b) shows a homo-monomeric structure of this embodiment of a vaccine delivery scaffold of the invention (right) compared to a conventional IgG heavy chain (left). In this embodiment, polymerisation of the fusion polypeptide is further enhanced by insertion of the IgM tail piece (m-tp) at the C-terminal end of the fusion polypeptide. As can be seen in (c), this allows the fusion polypeptide to polymerise through the Fc component, in addition to the CTB sequences, as shown in the embodiment of FIG. 1A.

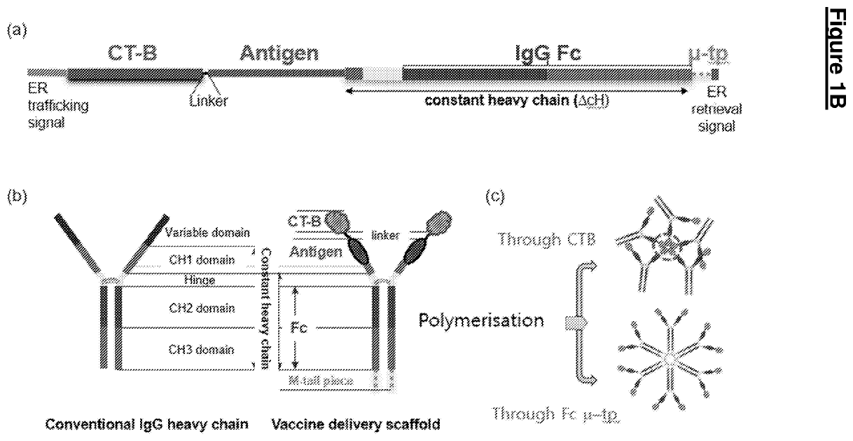

FIG. 2 shows the expression and verification of three main components in an embodiment of the fusion polypeptide which behaves as a Dengue vaccine candidate. (a) shows the immuno-detection of the PCF fusion polypeptide probing by anti-IgG Fc, anti-dengue antigen and anti-CTB specific antibodies after SDS-PAGE gel electrophoresis under non-reducing (NR) and reducing (R) conditions, showing polymer (P) and single chain(S). (b) shows the effect of a short flexible GP (6 aa) peptide linker and a long rigid EK (17 aa) peptide linker, and was analyzed by Coomassie gel staining, showing similar levels of expression and assembly of PCF constructs. (c) is a Western blot showing the effect of the insertion of the IgM tail piece (m-tp) into the fusion polypeptide of the invention. Lane 1 shows the fusion polypeptide of the invention without m-tp (as exemplified in FIG. 1A); lane 2 shows the fusion polypeptide of the invention with m-tp (as exemplified in FIG. 1B). The intense signal of the bands observed in lane 2 reflects an improved expression and polymerisation of the fusion polypeptide by insertion of the IgM tail piece (m-tp). Lane 3 shows the fusion protein expressed without an ER-targeting sequence. The lack of any bands observed in lane 3 indicates the key role played by the ER-targeting sequence in the expression of the fusion polypeptide.

FIG. 3 discloses the functional characterization of the PCF fusion polypeptide of the invention. The biological activity of the Dengue PCF (D-PCF) fusion polypeptide is demonstrated by binding to GM1 ganglioside in (a). The binding to the surface of antigen-presenting cells is shown in (b), and recombinant low affinity FcδRIII in vitro is shown in (c). The capability of superior binding of D-PCF over control IgG is demonstrated in (a) and (c).

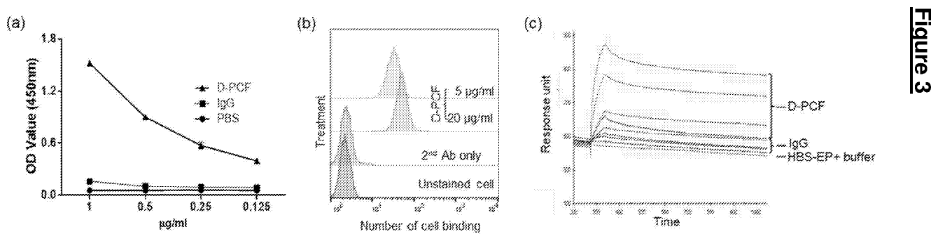

FIG. 4 shows humoral antibody responses in mice administered with the fusion polypeptide of the invention. The antigen-specific serum IgG and BAL IgA antibodies were induced in the immunized group with D-PCF after one systemic injection followed by two nasal boost applications. (a) shows the titres of IgG analysed in pooled sera. (b) shows IgG subtypes and IgA analysed for individual mice, with the X axis showing dilution points.

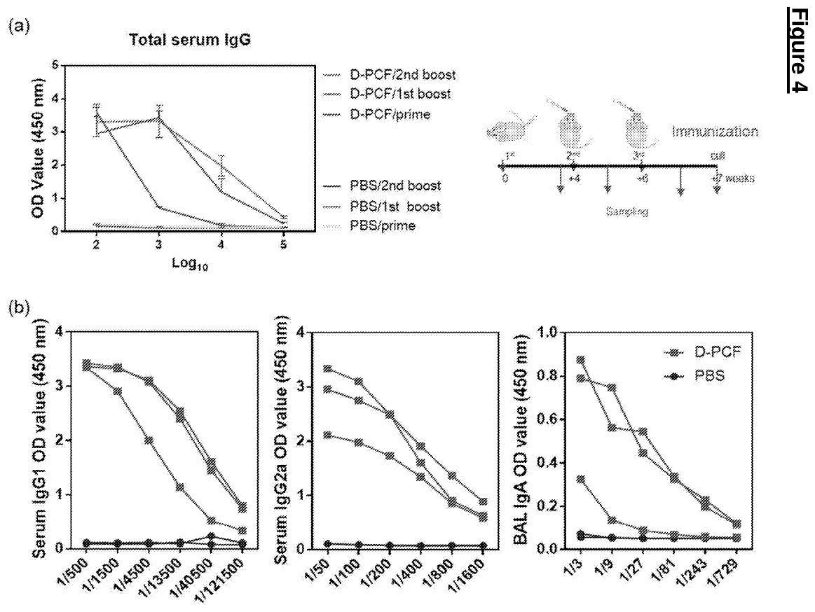

FIG. 5 shows cellular immune responses in the spleen of mice administered with the fusion polypeptide of the invention. To measure INF-8, IL-4 and IL-10, culture supernatants were diluted serially 1 in 3. For IL-17, the supernatants were diluted 1 in 2.

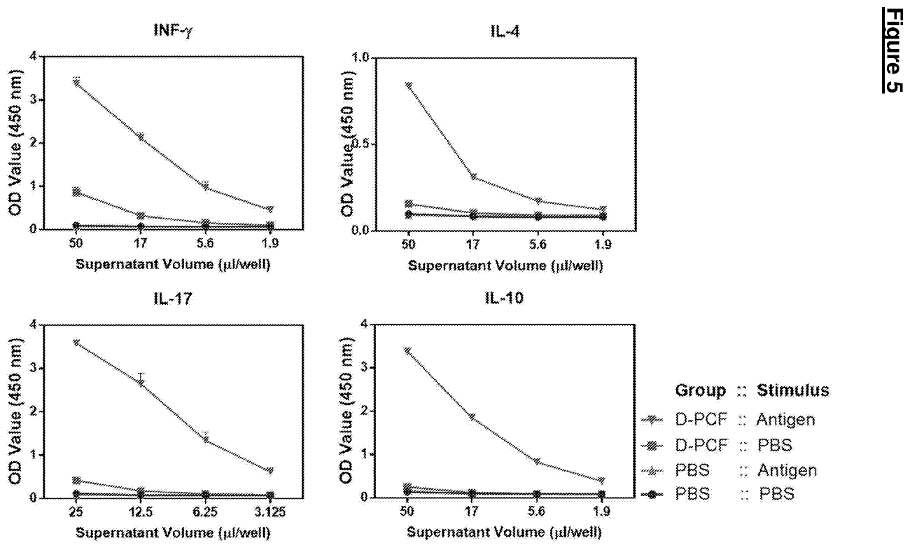

FIG. 6 shows the consistency of polymerization and functionality of three different PCF fusion polypeptides of the invention with Dengue, SARS CoV-2 and TB antigens. (a) shows immuno-detection probing by an anti-CT-B specific antibody under NR and R conditions. (b) shows protein analysis after SDS-PAGE and NativePAGE gel electrophoresis. (c) shows particle size distribution analysis by Zeta-sizer for monomeric IgG, pentameric IgM and of three different PCF fusion polypeptides of the invention with Dengue, SARS CoV-2 and TB antigens. It can be readily observed that the PCF fusion polypeptides are larger than the monomeric IgG, and are of a similar or larger size than the pentameric IgM, which therefore indicates their polymerisation. Red and green lines are repeat measurements, whereas the vertical lines indicate dominant peaks. (d) C1q complement and GM1 ganglioside binding analyses was performed to confirm the polymerization and biological activity of CTB, respectively. (e) Binding to surface of U937 cells, representing APC.

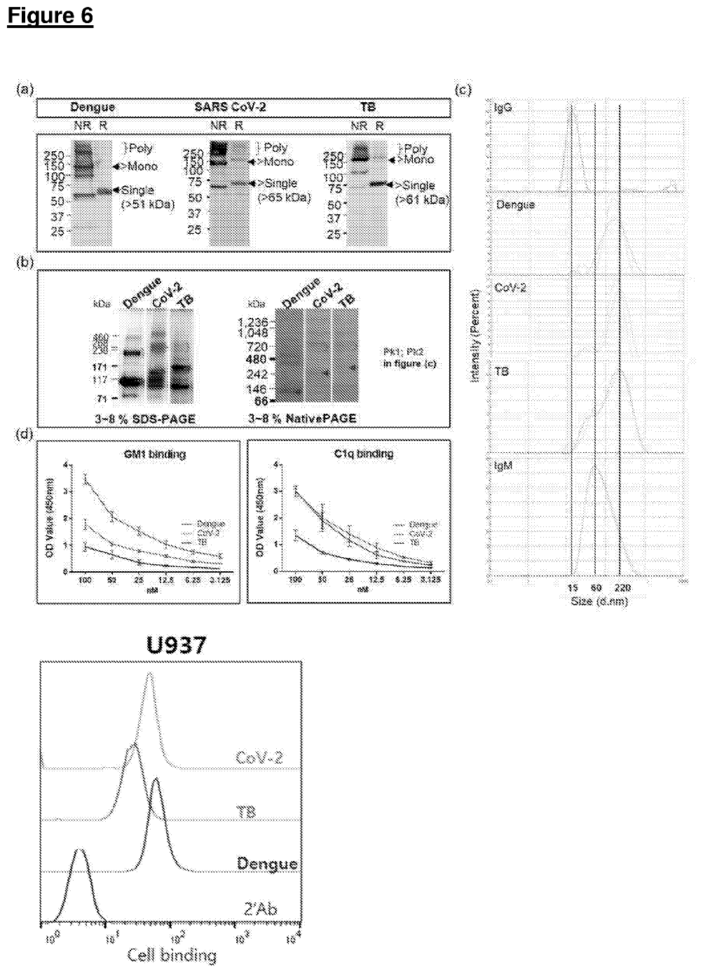

FIG. 7 shows that the fusion polypeptide (PCF) is non-toxic to human cells. THP-1 cells were incubated with control buffer saline solution (NC) or increasing concentrations of PCF over a 48 hour period, and cell viability determined at indicated time points. Reduction of viability observed at 48 hour is no different to negative control, and is due to exhaustion of nutrients.

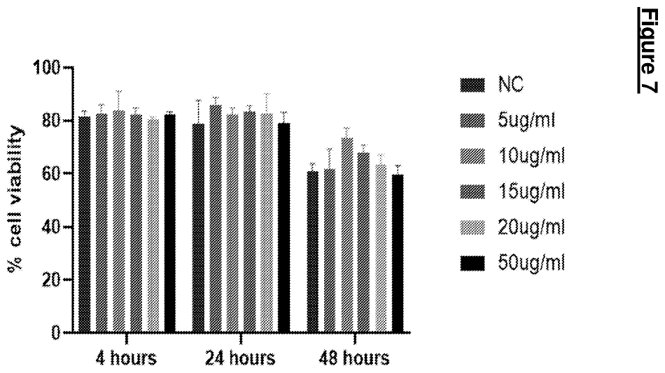

FIG. 8 presents the ability of the fusion polypeptide (PCF) to bind to primary human tonsillar mononuclear cells (TMC) by showing the representative flow cytometry histogram of PCF binding TMCs at a concentration of 20 μg/mL. A) represents the overall binding within the general TMCs population, with the combination of TMCs and a secondary antibody alone (sec. Ab) being the negative control. B) represents a histogram overlay showing human PCF binding specifically to CD14+ cells and the statistically different (Mean Fluorescent Intensity) MFI C) represents histogram overlay showing human PCF binding to CD19+ cells and the statistically different MFI. *p<0.05, **p<0.001. Conclusion: a moderate level of binding was observed with monocytes (B) but a greater degree of binding was observed with tonsillar B cells, which express Fc gamma receptors.

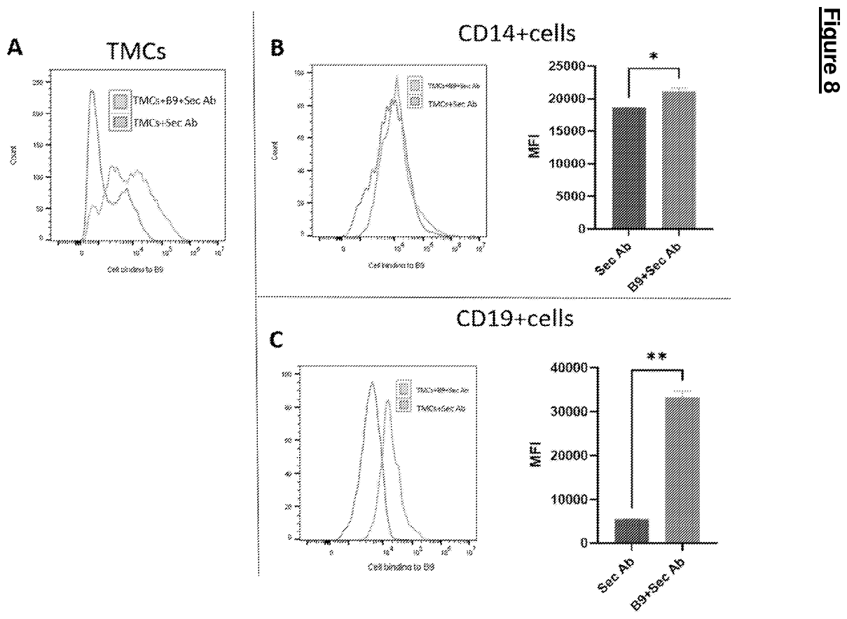

FIG. 9 demonstrates the feasibility of aerosolization of CoV2-PCF. Thus, 0.5 mg of PCF was aerosolised in Omron nebulizer and recovered protein from aerosol analysed after condensation. A) shows protein content before and after aerosolization, with or without addition of 0.05% Tween-80; B) binding to GM1 before and after aerosolization, with or without Tween-80; C) Binding to U937 cells before and after aerosolization, with Tween-80 included in aerosolization.

FIG. 10 shows the results of an ELISA test measuring the binding affinity of antibodies present in the sera of Covid-19 vaccinated human hosts (2 donors, blue and red) to RBD (SARS CoV-2 antigen) within the Cov2-PCF molecule, compared to RBD within the RBD-Fc construct or RBD alone. These results show the superior affinity of the serum antibodies to the polymeric RBD within the Cov2-PCF molecule compared to the monomeric forms of RBD-Fc or RBD alone. They also show that the RBD antigen is not masked within the polypeptide, which is a pre-requisite for recognition by B cells in vivo.

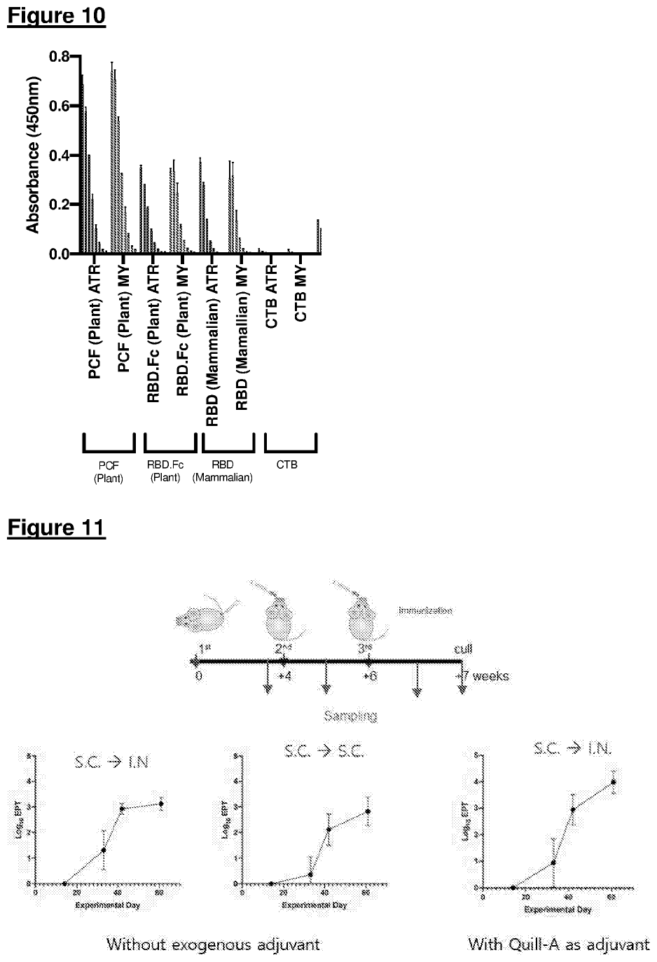

FIG. 11 shows the boosting effect of CoV2-PCF in mice previously primed by the systemic (s.c.) injection of CoV2-PCF. The ELISA results show serum RBD-specific antibody responses following intranasal and systemic boosting, with or without administration of an exogenous adjuvant (Quill-A).

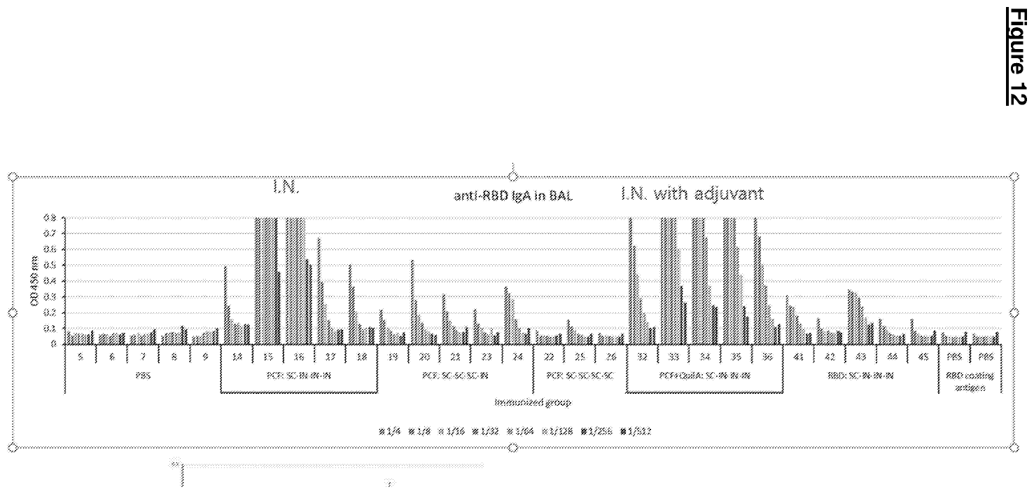

FIG. 12 shows the boosting effect of CoV2-PCF in mice previously primed by the systemic (s.c.) injection of CoV2-PCF. The ELISA results confirm RBD-specific IgA antibody responses in broncho-alveolar lavage following intranasal and systemic boosting, with or without an exogenous adjuvant (Quill-A).

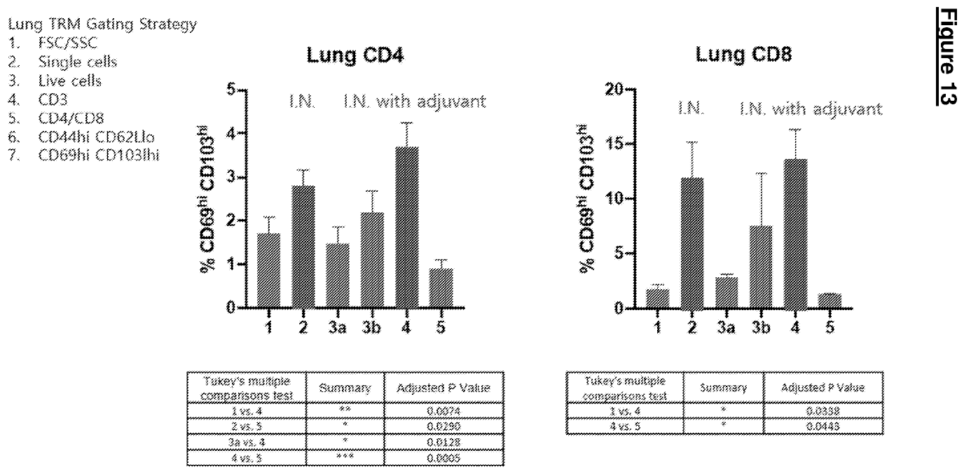

FIG. 13 shows the mucosal boosting effect of CoV2-PCF on lung resident memory T cells (Trm). These results demonstrate that only the mucosally boosted animals (red bars), but not the systemically injected or control animals (grey bars), generated significant CD4 and CD8 Trm levels.

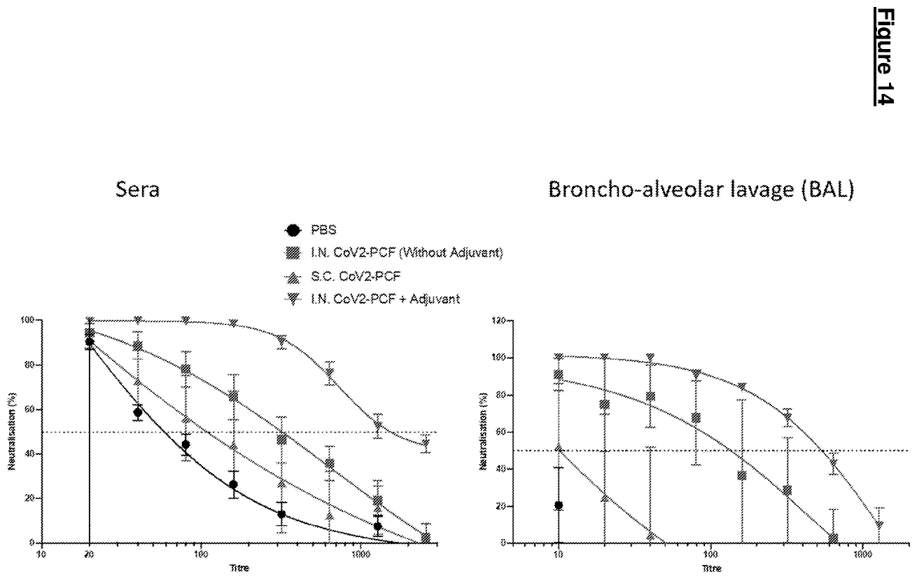

FIG. 14 shows HEK293T cells SARS-CoV2 neutralisation data by sera and broncho-alveolar lavage (BAL). The assay was performed with a pseudovirus (Wuhan strain) and luciferase-based readout. Individual points represent means from 5 mice per group, with the essay performed in duplicate for each mouse. The perforated line indicates 50% neutralisation.

### EXAMPLES

The inventors have designed and constructed a novel mucosal/systemic self-adjuvanting vaccine delivery platform, which they call “Platform CTB-Fc”, or “PCF”. The vaccine platform comprises a fusion protein having three key components, namely a non-toxic cholera toxin B subunit (CT-B), an antigen, and an Fc region from an Ig. However, it will be appreciated that the CTB is only an exemplary non-toxic B subunit of an AB₅ toxin, and that any other AB₅ toxin B subunit may be used in the immunogenic fusion polypeptide of the present invention.

ER to Golgi trafficking and/or ER retrieval signal peptides were employed to improve the level of expression and polymerization in a host cell, but are optional components of the fusion protein. Flexible or rigid linking between the antigen and the CTB were achieved by a GP or EK linker, and are also optional elements.

To demonstrate that the PCF vaccine of the invention could efficiently induce antigen-specific mucosal and systemic IgA and IgG antibodies and cell-mediated immune responses in human IgG receptor/CD64 transgenic mice in the absence of any exogenous adjuvants, a Dengue PCF (D-PCF) vaccine candidate was constructed as a proof of concept. D-PDF contained the Dengue virus envelop protein domain III consensus sequence (covering all four dengue virus serotypes) and displays double functionality, i.e. binding to mucosal surface GM1-gangliosides and Fc gamma receptors on antigen-presenting cells.

Additionally, PCF vaccine platforms directed to SARS CoV-2 and TB were also constructed, and their functionality demonstrated in vitro. The inventors also compared the antigen-antibody affinity of the polymeric fusion polypeptide of the invention to that of monomeric constructs.

In addition to the in vitro tests, the SARS-CoV-2-PCF vaccine was tested in mice. Antibody responses were assessed following systemic administration of the vaccine in mice cohorts and subsequent intranasal, mucosal, and systemic booster dosages. The efficacy of the vaccine construct was evaluated in the presence and the absence of exogenous adjuvants.

### Materials and Methods

### Structure for Vaccine Delivery Platform, i.e. “Platform CTB-Fc”, or “PCF” (PCF)

Referring first to FIG. 1A, there is shown a first embodiment of a fusion polypeptide (or protein) of the invention. As can be seen in FIG. 1A(a), the fusion polypeptide largely comprises three domains or peptides, namely (i) a cholera toxin B subunit (CTB), (ii) an antigen, and (iii) an IgG Fc region. These three domains can be arranged in any order in the fusion polypeptide, but in the embodiment shown in FIG. 1A(a), the CTB is disposed towards the N-terminus of the polypeptide with the IgG Fc region towards the C-terminus, and with the antigen in between the CTB and IgG Fc region. In some embodiments, the IgG Fc region can be disposed towards the N-terminus of the polypeptide with the CTB disposed towards the C-terminus, and with the antigen in between the CTB and IgG Fc region. In yet another embodiment, the position of the antigen can be either N-terminal, or C-terminal, and so on.

FIG. 1A(b) (left hand side) shows just the two heavy chains of a conventional IgG antibody (the light chains are not shown for simplicity), and how the IgG Fc region domain of the fusion polypeptide of the invention resembles it. In a conventional IgG heavy chain, there is a terminal variable domain (VH), and then the CH₁ domain, a hinge section, and then a CH₂—CH₃ domain. As shown in FIG. 1A(b), in the fusion polypeptide of the invention, a truncated sequence of IgG constant heavy chain is ideally used, which lacks a variable region of the heavy chain (VH) and includes only a partial C-terminal CH₁ domain for retaining the immunoglobulin-like Y shape, followed by a hinge-CH₂—CH₃ domain, but dispensing with the majority of the CH₁ domain. This is because, in some embodiments, much of the CH₁ domain can be considered unnecessary.

As shown in FIGS. 1A(b) and 1A(c), the single monomeric form (S) of the fusion polypeptide can dimerise to form a structure via the hinge section, which closely resembles the Y-shape of an IgG antibody. Then, as shown in FIG. 1A(c), this Y-shaped dimer of the polypeptide, can itself polymerise, whereby a plurality of polypeptide Y-shaped dimers aggregate, combine or fuse together. This aggregation or polymerization is achieved through the CTB domain. As can be seen in FIG. 1A(c), polymerization can create a monomer, dimer, trimer, tetramer, or pentamer of the Y-shaped dimerised fusion polypeptide. The pentameric structure shown in FIG. 1A(c) illustrates a ring around the respective CTB domains from each of the five fusion polypeptide dimers.

Two versions of this embodiment of the fusion polypeptide of the invention were constructed, one based on the truncated heavy chain of murine IgG (IgG2a) and the other based on the truncated heavy chain of human IgG (IgG1), for in vivo testing in mice or human, respectively. A point mutation (i.e. Ile221Asn) in the mouse IgG2a heavy chain can be inserted to prohibit protein cleavage by papain. A point mutation (i.e. Cys219Ser) can also be inserted to enable Fc-Fc pairing in the absence of the light chain.

Also as shown in FIG. 1A(a), in order to improve the level of expression and polymerization of the fusion polypeptide in a host cell, an ER to Golgi trafficking signal peptide was fused to the N-terminus of the polypeptide, which, as shown in the Figure, is CTB. Also, to further enhance expression and polymerization levels of the polypeptide, an ER retrieval signal peptide was included at the C-terminal end of the sequence, which, as shown, is the IgG Fc region. A 5′ untranslated region (5′ UTR) was optionally added at the upstream of the initiation codon, which is important for the regulation of translation of a transcript by differing mechanisms in viruses, prokaryotes and eukaryotes.

FIG. 1A(a) also shows that a linker sequence is disposed between the CTB sequence and the antigen sequence. For flexible or rigid linking between the antigen and CTB domains, either a flexible GP linker or a rigid EK linker was used in the fusion polypeptide, and disposed between the antigen and the CTB sequences. A rigid linker can be used as it was found to be resistant to proteolytic degradation, which is advantageous, but, in some embodiments, it may limit the ability of some structures that require polymeric assembly, as shown in FIG. 1A(c). Therefore, in some embodiments, a flexible linker can be used which does not prevent polymerization, and so the polymeric structures shown in FIG. 1A(c) can be created through CTB bonding.

The vaccine antigen sequence was inserted between the linker that follows CTB and the IgG Fc, by recombinant DNA technology. However, as discussed above, the positions of the antigen domain, the CTB domain and the IgG Fc region domains can be varied.

The gene sequence of one embodiment of a fusion polypeptide consisting of cholera toxin B subunit (CTB) and immunoglobulin IgG Fc was plant codon optimized and synthesized.

Referring now to FIG. 1B(a)-(c), there is a shown a second embodiment of the fusion polypeptide (or protein) of the invention. This embodiment is identical to the first embodiment shown in FIG. 1A except that the fusion protein additionally comprises a tailpiece “μ-tp” or “M-tail piece” of an immunoglobulin (for example, a human IgM-tail piece) towards the C-terminal end of the IgG Fc region of the fusion polypeptide. The “μ-tp” or “M-tail piece” serves to further improve the structural and polymerization characteristics of the fusion polypeptide, because it allows the fusion polypeptide to polymerise through the Fc component, in addition to CTB, as shown in FIG. 1B(c). As can be seen in FIG. 1B(c), polymerization can create a monomer, dimer, trimer, tetramer, or pentamer of the Y-shaped dimerised fusion polypeptide (when polymerizing through the CTB), and a hexamer (when polymerizing through the M-tail piece).

The relevant nucleotide sequences are listed in Table 1.

TABLE 1
Summary of nucleotide sequences used

| SEQ ID | | |
|---|---|---|
| No | Component | Sequence |
| 2 | CTB | ACACCACAGAACATCACAGATCTCTGCGCCGAGTACCACAACACCCAGATCCACAC |
| | | GCTCAACGACAAGATCTTCAGCTACACCGAGAGCCTCGCTGGCAAGCGCGAGATGG |
| | | CCATTATCACCTTCAAGAACGGCGCCACCTTCCAGGTTGAGGTGCCAGGCTCTCAG |
| | | CACATCGACTCCCAGAAGAAAGCCATCGAGCGCATGAAGGACACCCTCAGGATCGC |
| | | CTACCTCACCGAGGCCAAGGTTGAGAAGCTCTGCGTGTGGAACAACAAGACCCCGC |
| | | ATGCCATTGCCGCCATCTCTATGGCCAAT |
| 4 | CH3 domain | GGCTCCGTGAGAGCCCCGCAAGTGTATGTTCTTCCGCCGCCAGAGGAAGAGATGAC |
| | from a mouse | CAAGAAGCAAGTCACCCTGACCTGCATGGTGACCGACTTCATGCCAGAGGATATCT |
| | IgG2a | ACGTCGAATGGACCAACAACGGCAAGACCGAGCTGAACTACAAGAACACCGAGCC |
| | | GGTGCTCGACTCCGACGGCTCCTACTTCATGTACTCCAAGCTCCGCGTCGAGAAGA |
| | | AGAACTGGGTCGAGCGCAACTCCTACTCCTGCTCCGTTGTTCATGAGGGCCTCCAC |
| | | AACCACCACACCACCAAGTCTTTCTCCCGGCCAACTGGCAAG |
| 6 | CH3 domain | GGCCAGCCAAGAGAGCCACAGGTTTACACACTTCCGCCGTCCAGGGATGAGCTGAC |
| | from a human | CAAGAACCAGGTGTCCCTGACCTGCCTCGTGAAGGGCTTCTACCCATCCGATATCG |
| | IgG1 | CCGTCGAGTGGGAGTCCAATGGCCAGCCTGAGAACAATTACAAGACCACACCGCCG |
| | | GTGCTCGACTCCGATGGCTCATTCTTCCTGTACTCCAAGCTGACCGTCGACAAATCC |
| | | AGATGGCAACAGGGCAACGTGTTCTCCTGCTCCGTTATGCACGAGGCCCTCCACAA |
| | | CCACTACACCCAGAAGTCTCTCAGCCTCTCGCCAGGCAAG |
| 8 | CH2 domain | GCGCCAAATCTTCTTGGCGGCCCATCCGTGTTCATCTTCCCGCCGAAGATCAAGGA |
| | from a mouse | CGTGCTCATGATCTCCCTCTCGCCGATCGTGACATGCGTGGTGGTGGATGTGTCCG |
| | IgG2a | AGGACGATCCGGATGTCCAGATCTCCTGGTTCGTGAACAACGTCGAGGTGCACACC |
| | | GCGCAGACACAAACACACCGCGAGGACTACAATAGCACCCTCAGAGTGGTGAGCGC |
| | | CCTGCCAATCCAGCACCAGGATTGGATGTCCGGGAAAGAATTCAAGTGCAAGGTCA |
| | | ACAACAAGGACCTGCCAGCGCCGATCGAGAGGACCATCTCTAAGCCAAAG |
| 10 | CH2 domain | GCTCCAGAACTTCTTGGCGGCCCATCCGTGTTTCTGTTCCCGCCGAAGCCAAAGGA |
| | from a human | CACGCTGATGATCTCTCGCACCCCAGAGGTGACATGCGTGGTGGTGGATGTGTCCC |
| | IgG1 | ACGAGGACCCTGAGGTGAAGTTCAACTGGTATGTGGACGGCGTCGAGGTGCACAA |
| | | CGCCAAGACAAAGCCGCGCGAGGAACAGTACAACTCCACCTACAGAGTGGTGTCCG |
| | | TGCTCACCGTGCTCCACCAGGATTGGCTGAACGGGAAAGAATACAAATGCAAGGTG |
| | | TCCAACAAGGCGCTCCCAGCGCCGATCGAAAAGACCATCTCTAAGGCGAAG |
| 12 | Hinge region | GAGCCAAGGGGCCCGACAAACAAGCCTTCTCCACCATGCAAGTGCCCA |
| | from a mouse | |
| | IgG2a (with | |
| | point mutation) | |
| 14 | Hinge region | GAGCCGAAGTCCTCCGACAAGACCCATACTTGCCCACCGTGTCCA |
| | from a human | |
| | IgG1 (with point | |
| | mutation) | |
| 16 | Truncated CH1 | GCCAGCTCTACCAAGGTGGACAAGAAGATC |
| | domain from a | |
| | mouse IgG2a | |
| 18 | Truncated CH1 | GCCTCCAATACCAAGGTGGACAAGAAGGTC |
| | domain from a | |
| | human IgG1 | |
| 20 | Dengue antigen | AAGGGCATGTCCTACGCTATGTGCACCGGCAAGTTCAAGTTGGAGAAGGAGGTGG |
| | | CTGAGACCCAGCACGGCACCATCTTGATCAAGGTGAAGTACGAGGGCGATGGCGC |
| | | TCCTTGCAAGATCCCTTTCGAGATCCAGGATGTGGAGAAGAAGCACGTGAACGGAA |
| | | GGTTGATCACCGCTAACCCTATCGTGACCGATAAGGAGTCCCCTGTGAACATCGAG |
| | | GCTGAGCCTCCTTTCGGCGATTCCTACATCGTGATCGGCGTGGGCGATAAGGCTTT |
| | | GAAGTTGAACTGGTTCAAGAAGGGTTCTTCA |
| 22 | SARS CoV-2 | CAGCCAACCGAATCCATTGTGCGCTTCCCGAACATCACCAATCTGTGCCCATTCGGC |
| | antigen | GAGGTGTTCAACGCCACAAGATTCGCCTCTGTGTACGCCTGGAACCGCAAGCGCAT |
| | | CTCCAATTGCGTGGCCGACTACTCCGTGCTCTACAACTCTGCCAGCTTCTCCACGTT |
| | | CAAGTGCTACGGCGTGTCCCCGACCAAGCTGAACGATCTCTGCTTCACCAACGTGT |
| | | ACGCCGACTCCTTCGTGATCAGAGGCGACGAGGTGAGGCAAATTGCCCCAGGCCA |
| | | GACAGGCAAGATCGCCGACTACAACTACAAGCTCCCGGACGACTTCACCGGCTGCG |
| | | TGATCGCTTGGAACTCCAACAACCTCGACTCCAAGGTCGGCGGCAACTACAATTAC |
| | | CTCTACCGCCTCTTCCGCAAGTCCAACCTCAAGCCGTTCGAGCGCGATATCTCCACC |
| | | GAGATCTATCAGGCCGGAAGCACCCCATGCAATGGCGTCGAAGGCTTCAACTGCTA |
| | | CTTCCCGCTCCAGTCCTACGGCTTCCAGCCTACAAATGGCGTGGGCTACCAACCGT |
| | | ACAGGGTCGTCGTTCTCAGCTTCGAGCTGCTCCATGCTCCA |
| 24 | TB antigen | ATGACTGAGCAGCAGTGGAATTTTGCTGGTATTGAGGCTGCTGCTTCTGCTATTCA |
| | | GGGTAATGTTACTTCTATTCATTCTCTTCTTGATGAGGGTAAACAGTCTCTTACTAA |
| | | GTTGGCTGCTGCTTGGGGTGGTTCTGGTTCTGAGGCTTACCAGGGTGTTCAGCAGA |
| | | AGTGGGATGCTACTGCTACTGAGCTTAACAATGCTCTTCAGAATCTTGCTAGGACT |
| | | ATTTCTGAGGCTGGTCAGGCTATGGCTTCTACTGAGGGTAATGTTACTGGTATGTT |
| | | TGCTGGTGGTGGTGGTGGTATGGCTGAGATGAAGACTGATGCTGCTACTCTTGCTC |
| | | AGGAGGCTGGTAATTTTGAGAGGATTTCTGGAGATTTGAAGACTCAGATTGATCAG |
| | | GTTGAGTCTACTGCTGGTTCTCTTCAGGGTCAGTGGAGGGGTGCTGCTGGTACTGC |
| | | TGCTCAGGCTGCTGTTGTTAGGTTTCAGGAGGCTGCTAATAAGCAGAAGCAGGAGC |
| | | TTGATGAGATTTCTACTAATATTAGGCAGGCTGGTGTTCAGTACTCTAGGGCTGAT |
| | | GAGGAGCAGCAGCAGGCTCTTTCTTCTCAGATGGGTTTT |
| 26 | ER targeting | ATGGAACTCGGCCTTTCTTGGATTTTCTTGCTCGCTATCCTCAAGGGCGTGCAATGC |
| | signal from a | |
| | human IgG1 | |
| 28 | ER targeting | ATGAAGAATACCTCCAGCCTCTGCCTCCTCCTCCTGGTGGTTCTCTGCTCCCTCACA |
| | signal from a | TGCAATTCCGGCCAGGCC |
| | rice amylase 3D | |
| 30 | ER retention | TCCGAGAAGGATGAGCTT |
| | signal | |
| 32 | GP linker | GGCCCAGGCCCGGGATCC |
| 34 | EK linker | GCCGAGGCCGCTGCTAAAGAGGCTGCCGCCAAAGAAGCTGCTGCCAAGGCT |
| 46 | IgM tail piece | CCAACTCTCTACAACGTGTCCCTCGTGATGTCTGATACTGCTGGCACTTGCTAC |
| | (human) | |
| 39 | Dengue fusion | ATGGAACTCGGCCTTTCTTGGATTTTCTTGCTCGCTATCCTCAAGGGCGTGCAATGC |
| | polypeptide | ACACCTCAAAATATTACTGATTTGTGTGCAGAATACCACAACACACAAATACATACG |
| | | CTAAATGATAAGATATTTTCGTATACAGAATCTCTAGCTGGAAAAAGAGAGATGGC |
| | | TATCATTACTTTTAAGAATGGTGCGACTTTTCAAGTAGAAGTACCAGGTAGTCAAC |
| | | ATATAGATTCACAAAAAAAAGCGATTGAAAGGATGAAGGATACCCTGAGGATTGCA |
| | | TATCTTACTGAAGCTAAAGTCGAAAAGTTATGTGTATGGAATAATAAAACGCCTCA |
| | | TGCGATTGCCGCAATTAGTATGGCAAATGGCCCAGGCCCGGGATCCAAGGGCATGT |
| | | CCTACGCTATGTGCACCGGCAAGTTCAAGTTGGAGAAGGAGGTGGCTGAGACCCA |
| | | GCACGGCACCATCTTGATCAAGGTGAAGTACGAGGGCGATGGCGCTCCTTGCAAGA |
| | | TCCCTTTCGAGATCCAGGATGTGGAGAAGAAGCACGTGAACGGAAGGTTGATCACC |
| | | GCTAACCCTATCGTGACCGATAAGGAGTCCCCTGTGAACATCGAGGCTGAGCCTCC |
| | | TTTCGGCGATTCCTACATCGTGATCGGCGTGGGCGATAAGGCTTTGAAGTTGAACT |
| | | GGTTCAAGAAGGGTTCATCTactagtGCTTCCAACACAAAAGTTGATAAGAAGGTTGA |
| | | GCCAAAGTCCTCCGATAAGACTCACACTTGTCCACCATGTCCAGCTCCAGAACTTCT |
| | | TGGAGGACCATCCGTTTTCTTGTTCCCACCAAAGCCAAAGGATACTCTCATGATCTC |
| | | CAGGACTCCAGAGGTTACATGCGTTGTGGTTGATGTGTCTCACGAGGATCCTGAGG |
| | | TGAAGTTCAACTGGTATGTGGATGGTGTTGAGGTGCACAACGCTAAGACTAAGCCA |
| | | CGTGAGGAACAGTACAACTCCACTTACAGGGTGGTGTCTGTGCTTACTGTGCTTCA |
| | | CCAGGATTGGCTTAACGGCAAAGAGTACAAGTGCAAGGTGTCCAACAAGGCTCTCC |
| | | CAGCTCCAATCGAAAAGACTATCTCCAAGGCTAAGGGCCAGCCAAGAGAGCCACAA |
| | | GTTTACACTCTTCCACCATCCAGGGATGAGCTTACTAAGAACCAGGTGTCCCTTACT |
| | | TGCCTCGTGAAGGGATTCTACCCATCCGATATTGCTGTTGAGTGGGAGTCTAATGG |
| | | CCAGCCTGAGAACAACTACAAGACTACTCCACCAGTGCTCGATTCCGATGGCTCATT |
| | | CTTCTTGTACTCCAAGCTCACTGTGGATAAGTCCAGGTGGCAGCAGGGAAACGTTT |
| | | TCTCTTGCTCTGTTATGCACGAGGCTCTCCACAACCACTACACTCAGAAGTCCCTCT |
| | | CACTCTCTACTGGCAAGTCCGAAAAGGATGAACTTTAA |
| 40 | SARS CoV-2 | ATGGAACTCGGCCTTTCTTGGATTTTCTTGCTCGCTATCCTCAAGGGCGTGCAATGC |
| | fusion | ACACCTCAAAATATTACTGATTTGTGTGCAGAATACCACAACACACAAATACATACG |
| | polypeptide | CTAAATGATAAGATATTTTCGTATACAGAATCTCTAGCTGGAAAAAGAGAGATGGC |
| | (mouse IgG2a | TATCATTACTTTTAAGAATGGTGCGACTTTTCAAGTAGAAGTACCAGGTAGTCAAC |
| | based) | ATATAGATTCACAAAAAAAAGCGATTGAAAGGATGAAGGATACCCTGAGGATTGCA |
| | | TATCTTACTGAAGCTAAAGTCGAAAAGTTATGTGTATGGAATAATAAAACGCCTCA |
| | | TGCGATTGCCGCAATTAGTATGGCAAATGGCCCAGGCCCGGGATCCCAGCCAACCG |
| | | AATCCATTGTGCGCTTCCCGAACATCACCAATCTGTGCCCATTCGGCGAGGTGTTCA |
| | | ACGCCACAAGATTCGCCTCTGTGTACGCCTGGAACCGCAAGCGCATCTCCAATTGC |
| | | GTGGCCGACTACTCCGTGCTCTACAACTCTGCCAGCTTCTCCACGTTCAAGTGCTAC |
| | | GGCGTGTCCCCGACCAAGCTGAACGATCTCTGCTTCACCAACGTGTACGCCGACTC |
| | | CTTCGTGATCAGAGGCGACGAGGTGAGGCAAATTGCCCCAGGCCAGACAGGCAAG |
| | | ATCGCCGACTACAACTACAAGCTCCCGGACGACTTCACCGGCTGCGTGATCGCTTG |
| | | GAACTCCAACAACCTCGACTCCAAGGTCGGCGGCAACTACAATTACCTCTACCGCCT |
| | | CTTCCGCAAGTCCAACCTCAAGCCGTTCGAGCGCGATATCTCCACCGAGATCTATCA |
| | | GGCCGGAAGCACCCCATGCAATGGCGTCGAAGGCTTCAACTGCTACTTCCCGCTCC |
| | | AGTCCTACGGCTTCCAGCCTACAAATGGCGTGGGCTACCAACCGTACAGGGTCGTC |
| | | GTTCTCAGCTTCGAGCTGCTCCATGCTCCAGCCAGCTCTACCAAGGTGGACAAGAA |
| | | GATCGAGCCAAGGGGCCCGACAAACAAGCCTTCTCCACCATGCAAGTGCCCAGCGC |
| | | CAAATCTTCTTGGCGGCCCATCCGTGTTCATCTTCCCGCCGAAGATCAAGGACGTG |
| | | CTCATGATCTCCCTCTCGCCGATCGTGACATGCGTGGTGGTGGATGTGTCCGAGGA |
| | | CGATCCGGATGTCCAGATCTCCTGGTTCGTGAACAACGTCGAGGTGCACACCGCGC |
| | | AGACACAAACACACCGCGAGGACTACAATAGCACCCTCAGAGTGGTGAGCGCCCTG |
| | | CCAATCCAGCACCAGGATTGGATGTCCGGGAAAGAATTCAAGTGCAAGGTCAACAA |
| | | CAAGGACCTGCCAGCGCCGATCGAGAGGACCATCTCTAAGCCAAAGGGCTCCGTGA |
| | | GAGCCCCGCAAGTGTATGTTCTTCCGCCGCCAGAGGAAGAGATGACCAAGAAGCAA |
| | | GTCACCCTGACCTGCATGGTGACCGACTTCATGCCAGAGGATATCTACGTCGAATG |
| | | GACCAACAACGGCAAGACCGAGCTGAACTACAAGAACACCGAGCCGGTGCTCGACT |
| | | CCGACGGCTCCTACTTCATGTACTCCAAGCTCCGCGTCGAGAAGAAGAACTGGGTC |
| | | GAGCGCAACTCCTACTCCTGCTCCGTTGTTCATGAGGGCCTCCACAACCACCACACC |
| | | ACCAAGTCTTTCTCCCGGCCAACTGGCAAGTCCGAGAAGGATGAGCTTTGA |
| 41 | SARS CoV-2 | ATGAAGAATACCTCCAGCCTCTGCCTCCTCCTCCTGGTGGTTCTCTGCTCCCTCACA |
| | fusion | TGCAATTCCGGCCAGGCCACACCACAGAACATCACAGATCTCTGCGCCGAGTACCA |
| | polypeptide | CAACACCCAGATCCACACGCTCAACGACAAGATCTTCAGCTACACCGAGAGCCTCG |
| | (human IgG1 | CTGGCAAGCGCGAGATGGCCATTATCACCTTCAAGAACGGCGCCACCTTCCAGGTT |
| | based) | GAGGTGCCAGGCTCTCAGCACATCGACTCCCAGAAGAAAGCCATCGAGCGCATGAA |
| | | GGACACCCTCAGGATCGCCTACCTCACCGAGGCCAAGGTTGAGAAGCTCTGCGTGT |
| | | GGAACAACAAGACCCCGCATGCCATTGCCGCCATCTCTATGGCCAATGCCGAGGCC |
| | | GCTGCTAAAGAGGCTGCCGCCAAAGAAGCTGCTGCCAAGGCTCAGCCAACCGAATC |
| | | CATTGTGCGCTTCCCGAACATCACCAATCTGTGCCCATTCGGCGAGGTGTTCAACG |
| | | CCACAAGATTCGCCTCTGTGTACGCCTGGAACCGCAAGCGCATCTCCAATTGCGTG |
| | | GCCGACTACTCCGTGCTCTACAACTCTGCCAGCTTCTCCACGTTCAAGTGCTACGGC |
| | | GTGTCCCCGACCAAGCTGAACGATCTCTGCTTCACCAACGTGTACGCCGACTCCTTC |
| | | GTGATCAGAGGCGACGAGGTGAGGCAAATTGCCCCAGGCCAGACAGGCAAGATCG |
| | | CCGACTACAACTACAAGCTCCCGGACGACTTCACCGGCTGCGTGATCGCTTGGAAC |
| | | TCCAACAACCTCGACTCCAAGGTCGGCGGCAACTACAATTACCTCTACCGCCTCTTC |
| | | CGCAAGTCCAACCTCAAGCCGTTCGAGCGCGATATCTCCACCGAGATCTATCAGGC |
| | | CGGAAGCACCCCATGCAATGGCGTCGAAGGCTTCAACTGCTACTTCCCGCTCCAGT |
| | | CCTACGGCTTCCAGCCTACAAATGGCGTGGGCTACCAACCGTACAGGGTCGTCGTT |
| | | CTCAGCTTCGAGCTGCTCCATGCGCCAGCCTCCAATACCAAGGTGGACAAGAAGGT |
| | | CGAGCCGAAGTCCTCCGACAAGACCCATACTTGCCCACCGTGTCCAGCTCCAGAAC |
| | | TTCTTGGCGGCCCATCCGTGTTTCTGTTCCCGCCGAAGCCAAAGGACACGCTGATG |
| | | ATCTCTCGCACCCCAGAGGTGACATGCGTGGTGGTGGATGTGTCCCACGAGGACCC |
| | | TGAGGTGAAGTTCAACTGGTATGTGGACGGCGTCGAGGTGCACAACGCCAAGACA |
| | | AAGCCGCGCGAGGAACAGTACAACTCCACCTACAGAGTGGTGTCCGTGCTCACCGT |
| | | GCTCCACCAGGATTGGCTGAACGGGAAAGAATACAAATGCAAGGTGTCCAACAAG |
| | | GCGCTCCCAGCGCCGATCGAAAAGACCATCTCTAAGGCGAAGGGCCAGCCAAGAG |
| | | AGCCACAGGTTTACACACTTCCGCCGTCCAGGGATGAGCTGACCAAGAACCAGGTG |
| | | TCCCTGACCTGCCTCGTGAAGGGCTTCTACCCATCCGATATCGCCGTCGAGTGGGA |
| | | GTCCAATGGCCAGCCTGAGAACAATTACAAGACCACACCGCCGGTGCTCGACTCCG |
| | | ATGGCTCATTCTTCCTGTACTCCAAGCTGACCGTCGACAAATCCAGATGGCAACAG |
| | | GGCAACGTGTTCTCCTGCTCCGTTATGCACGAGGCCCTCCACAACCACTACACCCAG |
| | | AAGTCTCTCAGCCTCTCGCCAGGCAAGTCCGAGAAGGATGAGCTTTGA |
| 42 | TB fusion | ATGGAACTCGGCCTTTCTTGGATTTTCTTGCTCGCTATCCTCAAGGGCGTGCAATGC |
| | polypeptide | ACACCTCAAAATATTACTGATTTGTGTGCAGAATACCACAACACACAAATACATACG |
| | | CTAAATGATAAGATATTTTCGTATACAGAATCTCTAGCTGGAAAAAGAGAGATGGC |
| | | TATCATTACTTTTAAGAATGGTGCGACTTTTCAAGTAGAAGTACCAGGTAGTCAAC |
| | | ATATAGATTCACAAAAAAAAGCGATTGAAAGGATGAAGGATACCCTGAGGATTGCA |
| | | TATCTTACTGAAGCTAAAGTCGAAAAGTTATGTGTATGGAATAATAAAACGCCTCA |
| | | TGCGATTGCCGCAATTAGTATGGCAAATGGCCCAGGCCCGggatccATGACTGAGCA |
| | | GCAGTGGAATTTTGCTGGTATTGAGGCTGCTGCTTCTGCTATTCAGGGTAATGTTA |
| | | CTTCTATTCATTCTCTTCTTGATGAGGGTAAACAGTCTCTTACTAAGTTGGCTGCTG |
| | | CTTGGGGTGGTTCTGGTTCTGAGGCTTACCAGGGTGTTCAGCAGAAGTGGGATGCT |
| | | ACTGCTACTGAGCTTAACAATGCTCTTCAGAATCTTGCTAGGACTATTTCTGAGGCT |
| | | GGTCAGGCTATGGCTTCTACTGAGGGTAATGTTACTGGTATGTTTGCTGGTGGTGG |
| | | TGGTGGTATGGCTGAGATGAAGACTGATGCTGCTACTCTTGCTCAGGAGGCTGGTA |
| | | ATTTTGAGAGGATTTCTGGAGATTTGAAGACTCAGATTGATCAGGTTGAGTCTACT |
| | | GCTGGTTCTCTTCAGGGTCAGTGGAGGGGTGCTGCTGGTACTGCTGCTCAGGCTGC |
| | | TGTTGTTAGGTTTCAGGAGGCTGCTAATAAGCAGAAGCAGGAGCTTGATGAGATTT |
| | | CTACTAATATTAGGCAGGCTGGTGTTCAGTACTCTAGGGCTGATGAGGAGCAGCAG |
| | | CAGGCTCTTTCTTCTCAGATGGGTTTTactagtGCTTCCAACACAAAAGTTGATAAGAA |
| | | GGTTGAGCCAAAGTCCTCCGATAAGACTCACACTTGTCCACCATGTCCAGCTCCAG |
| | | AACTTCTTGGAGGACCATCCGTTTTCTTGTTCCCACCAAAGCCAAAGGATACTCTCA |
| | | TGATCTCCAGGACTCCAGAGGTTACATGCGTTGTGGTTGATGTGTCTCACGAGGAT |
| | | CCTGAGGTGAAGTTCAACTGGTATGTGGATGGTGTTGAGGTGCACAACGCTAAGAC |
| | | TAAGCCACGTGAGGAACAGTACAACTCCACTTACAGGGTGGTGTCTGTGCTTACTG |
| | | TGCTTCACCAGGATTGGCTTAACGGCAAAGAGTACAAGTGCAAGGTGTCCAACAAG |
| | | GCTCTCCCAGCTCCAATCGAAAAGACTATCTCCAAGGCTAAGGGCCAGCCAAGAGA |
| | | GCCACAAGTTTACACTCTTCCACCATCCAGGGATGAGCTTACTAAGAACCAGGTGT |
| | | CCCTTACTTGCCTCGTGAAGGGATTCTACCCATCCGATATTGCTGTTGAGTGGGAG |
| | | TCTAATGGCCAGCCTGAGAACAACTACAAGACTACTCCACCAGTGCTCGATTCCGA |
| | | TGGCTCATTCTTCTTGTACTCCAAGCTCACTGTGGATAAGTCCAGGTGGCAGCAGG |
| | | GAAACGTTTTCTCTTGCTCTGTTATGCACGAGGCTCTCCACAACCACTACACTCAGA |
| | | AGTCCCTCTCACTCTCTACTGGCAAGTCCGAAAAGGATGAACTTTAA |
| 52 | Human CoV2- | ATGAAGAATACCTCCAGCCTCTGCCTCCTCCTCCTGGTGGTTCTCTGCTCCCTCACA |
| | PCF including | TGCAATTCCGGCCAGGCCACACCACAGAACATCACAGATCTCTGCGCCGAGTACCA |
| | IgM tail piece | CAACACCCAGATCCACACGCTCAACGACAAGATCTTCAGCTACACCGAGAGCCTCG |
| | | CTGGCAAGCGCGAGATGGCCATTATCACCTTCAAGAACGGCGCCACCTTCCAGGTT |
| | | GAGGTGCCAGGCTCTCAGCACATCGACTCCCAGAAGAAAGCCATCGAGCGCATGAA |
| | | GGACACCCTCAGGATCGCCTACCTCACCGAGGCCAAGGTTGAGAAGCTCTGCGTGT |
| | | GGAACAACAAGACCCCGCATGCCATTGCCGCCATCTCTATGGCCAATGCCGAGGCC |
| | | GCTGCTAAAGAGGCTGCCGCCAAAGAAGCTGCTGCCAAGGCTCAGCCAACCGAATC |
| | | CATTGTGCGCTTCCCGAACATCACCAATCTGTGCCCATTCGGCGAGGTGTTCAACG |
| | | CCACAAGATTCGCCTCTGTGTACGCCTGGAACCGCAAGCGCATCTCCAATTGCGTG |
| | | GCCGACTACTCCGTGCTCTACAACTCTGCCAGCTTCTCCACGTTCAAGTGCTACGGC |
| | | GTGTCCCCGACCAAGCTGAACGATCTCTGCTTCACCAACGTGTACGCCGACTCCTTC |
| | | GTGATCAGAGGCGACGAGGTGAGGCAAATTGCCCCAGGCCAGACAGGCAAGATCG |
| | | CCGACTACAACTACAAGCTCCCGGACGACTTCACCGGCTGCGTGATCGCTTGGAAC |
| | | TCCAACAACCTCGACTCCAAGGTCGGCGGCAACTACAATTACCTCTACCGCCTCTTC |
| | | CGCAAGTCCAACCTCAAGCCGTTCGAGCGCGATATCTCCACCGAGATCTATCAGGC |
| | | CGGAAGCACCCCATGCAATGGCGTCGAAGGCTTCAACTGCTACTTCCCGCTCCAGT |
| | | CCTACGGCTTCCAGCCTACAAATGGCGTGGGCTACCAACCGTACAGGGTCGTCGTT |
| | | CTCAGCTTCGAGCTGCTCCATGCGCCAGCCTCCAATACCAAGGTGGACAAGAAGGT |
| | | CGAGCCGAAGTCCTCCGACAAGACCCATACTTGCCCACCGTGTCCAGCTCCAGAAC |
| | | TTCTTGGCGGCCCATCCGTGTTTCTGTTCCCGCCGAAGCCAAAGGACACGCTGATG |
| | | ATCTCTCGCACCCCAGAGGTGACATGCGTGGTGGTGGATGTGTCCCACGAGGACCC |
| | | TGAGGTGAAGTTCAACTGGTATGTGGACGGCGTCGAGGTGCACAACGCCAAGACA |
| | | AAGCCGCGCGAGGAACAGTACAACTCCACCTACAGAGTGGTGTCCGTGCTCACCGT |
| | | GCTCCACCAGGATTGGCTGAACGGGAAAGAATACAAATGCAAGGTGTCCAACAAG |
| | | GCGCTCCCAGCGCCGATCGAAAAGACCATCTCTAAGGCGAAGGGCCAGCCAAGAG |
| | | AGCCACAGGTTTACACACTTCCGCCGTCCAGGGATGAGCTGACCAAGAACCAGGTG |
| | | TCCCTGACCTGCCTCGTGAAGGGCTTCTACCCATCCGATATCGCCGTCGAGTGGGA |
| | | GTCCAATGGCCAGCCTGAGAACAATTACAAGACCACACCGCCGGTGCTCGACTCCG |
| | | ATGGCTCATTCTTCCTGTACTCCAAGCTGACCGTCGACAAGTCCAGGTGGCAGCAG |
| | | GGAAACGTTTTCTCTTGCTCTGTTATGCACGAGGCTCTCCACAACCACTACACTCAG |
| | | AAGTCCCTCTCACTCTCTACTGGCAAGCCAACTCTCTACAACGTGTCCCTCGTGATG |
| | | TCTGATACTGCTGGCACTTGCTACTCCGAAAAGGATGAACTTTAA |

The related amino acid sequences are shown in Table 2.

TABLE 2
Summary of amino acid sequences used

| SEQ ID | | |
|---|---|---|
| No | Component | Sequence |
| 1 | CTB | TPQNITDLCAEYHNTQIHTLNDKIFSYTESLAGKREMAIITFKNGATFQVEVPGSQHI |
| | | DSQKKAIERMKDTLRIAYLTEAKVEKLCVWNNKTPHAIAAISMAN |
| 3 | CH3 domain | GSVRAPQVYVLPPPEEEMTKKQVTLTCMVTDFMPEDIYVEWTNNGKTELNYKNTE |
| | from a mouse | PVLDSDGSYFMYSKLRVEKKNWVERNSYSCSVVHEGLHNHHTTKSFSRPTGK |
| | IgG2a | |
| 5 | CH 3 domain | GQPREPQVYTLPPSRDELTKNQVSLTCLVKGFYPSDIAVEWESNGQPENNYKTTPPV |
| | from a human | LDSDGSFFLYSKLTVDKSRWQQGNVFSCSVMHEALHNHYTQKSLSLSPGK |
| | IgG1 | |
| 7 | CH2 domain | APNLLGGPSVFIFPPKIKDVLMISLSPIVTCVVVDVSEDDPDVQISWFVNNVEVHTAQ |
| | from a mouse | TQTHREDYNSTLRVVSALPIQHQDWMSGKEFKCKVNNKDLPAPIERTISKPK |
| | IgG2a | |
| 9 | CH2 domain | APELLGGPSVFLFPPKPKDTLMISRTPEVTCVVVDVSHEDPEVKFNWYVDGVEVHN |
| | from a human | AKTKPREEQYNSTYRVVSVLTVLHQDWLNGKEYKCKVSNKALPAPIEKTISKAK |
| | IgG1 | |
| 11 | Hinge region | EPRGPTNKPSPPCKCP |
| | from a mouse | |
| | IgG2a (with | |
| | point mutation) | |
| 13 | Hinge region | EPKSSDKTHTCPPCP |
| | from a human | |
| | IgG1 (with point | |
| | mutation) | |
| 15 | Truncated CH1 | ASSTKVDKKI |
| | domain from a | |
| | mouse IgG2a | |
| 17 | Truncated CH1 | ASNTKVDKKV |
| | domain from a | |
| | human IgG1 | |
| 19 | Dengue antigen | KGMSYAMCTGKFKLEKEVAETQHGTILIKVKYEGDGAPCKIPFEIQDVEKKHVNGR |
| | | LITANPIVTDKESPVNIEAEPPFGDSYIVIGVGDKALKLNWFKKGSS |
| 21 | SARS CoV-2 | QPTESIVRFPNITNLCPFGEVFNATRFASVYAWNRKRISNCVADYSVLYNSASFSTFK |
| | antigen | CYGVSPTKLNDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGVEGFNCYFPL |
| | | QSYGFQPTNGVGYQPYRVVVLSFELLHAP |
| 23 | TB antigen | MTEQQWNFAGIEAAASAIQGNVTSIHSLLDEGKQSLTKLAAAWGGSGSEAYQGVQ |
| | | QKWDATATELNNALQNLARTISEAGQAMASTEGNVTGMFAGGGGGMAEMKTDA |
| | | ATLAQEAGNFERISGDLKTQIDQVESTAGSLQGQWRGAAGTAAQAAVVRFQEAAN |
| | | KQKQELDEISTNIRQAGVQYSRADEEQQQALSSQMGF |
| 25 | ER targeting | MELGLSWIFLLAILKGVQC |
| | signal from a | |
| | human IgG1 | |
| 27 | ER targeting | MKNTSSLCLLLLVVLCSLTCNSGQA |
| | signal from a | |
| | rice amylase 3D | |
| 29 | ER retention | SEKDEL |
| | signal | |
| 31 | GP linker | GPGPGS |
| 33 | EK linker | AEAAAKEAAAKEAAAKA |
| 45 | IgM tail piece | PTLYNVSLVMSDTAGTCY |
| | (human) | |
| 35 | Dengue fusion | MELGLSWIFLLAILKGVQCTPQNITDLCAEYHNTQIHTLNDKIFSYTESLAGKREMA |
| | polypeptide | IITFKNGATFQVEVPGSQHIDSQKKAIERMKDTLRIAYLTEAKVEKLCVWNNKTPHA |
| | | IAAISMANGPGPGSKGMSYAMCTGKFKLEKEVAETQHGTILIKVKYEGDGAPCKIPF |
| | | EIQDVEKKHVNGRLITANPIVTDKESPVNIEAEPPFGDSYIVIGVGDKALKLNWFKK |
| | | GSSTSASNTKVDKKVEPKSSDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQQGNVFSC |
| | | SVMHEALHNHYTQKSLSLSTGKSEKDEL |
| 36 | SARS CoV-2 | MELGLSWIFLLAILKGVQCTPQNITDLCAEYHNTQIHTLNDKIFSYTESLAGKREMA |
| | fusion | IITFKNGATFQVEVPGSQHIDSQKKAIERMKDTLRIAYLTEAKVEKLCVWNNKTPHA |
| | polypeptide | LAAISMANGPGPGSQPTESIVRFPNITNLCPFGEVFNATRFASVYAWNRKRISNCVA |
| | (mouse IgG2a | DYSVLYNSASFSTFKCYGVSPTKLNDLCFTNVYADSFVIRGDEVRQIAPGQTGKIADY |
| | based) | NYKLPDDFTGCVIAWNSNNLDSKVGGNYNYLYRLFRKSNLKPFERDISTEIYQAGST |
| | | PCNGVEGFNCYFPLQSYGFQPTNGVGYQPYRVVVLSFELLHAPASSTKVDKKIEPRG |
| | | PTNKPSPPCKCPAPNLLGGPSVFIFPPKIKDVLMISLSPIVTCVVVDVSEDDPDVQISW |
| | | FVNNVEVHTAQTQTHREDYNSTLRVVSALPIQHQDWMSGKEFKCKVNNKDLPAPI |
| | | ERTISKPKGSVRAPQVYVLPPPEEEMTKKQVTLTCMVTDFMPEDIYVEWTNNGKTE |
| | | LNYKNTEPVLDSDGSYFMYSKLRVEKKNWVERNSYSCSVVHEGLHNHHTTKSFSR |
| | | PTGKSEKDEL |
| 37 | SARS CoV-2 | MKNTSSLCLLLLVVLCSLTCNSGQATPQNITDLCAEYHNTQIHTLNDKIFSYTESLAG |
| | fusion | KREMAIITFKNGATFQVEVPGSQHIDSQKKAIERMKDTLRIAYLTEAKVEKLCVWN |
| | polypeptide | NKTPHAIAAISMANAEAAAKEAAAKEAAAKAQPTESIVRFPNITNLCPFGEVFNATR |
| | (human IgG1 | FASVYAWNRKRISNCVADYSVLYNSASFSTFKCYGVSPTKLNDLCFTNVYADSFVIRG |
| | based) | DEVRQIAPGQTGKIADYNYKLPDDFTGCVIAWNSNNLDSKVGGNYNYLYRLFRKSN |
| | | LKPFERDISTEIYQAGSTPCNGVEGFNCYFPLQSYGFQPTNGVGYQPYRVVVLSFELL |
| | | HAPASNTKVDKKVEPKSSDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFSCSV |
| | | MHEALHNHYTQKSLSLSPGKSEKDEL |
| 38 | TB fusion | MELGLSWIFLLAILKGVQCTPQNITDLCAEYHNTQIHTLNDKIFSYTESLAGKREMA |
| | polypeptide | IITFKNGATFQVEVPGSQHIDSQKKAIERMKDTLRIAYLTEAKVEKLCVWNNKTPHA |
| | | IAAISMANGPGPGSMTEQQWNFAGIEAAASAIQGNVTSIHSLLDEGKQSLTKLAAA |
| | | WGGSGSEAYQGVQQKWDATATELNNALQNLARTISEAGQAMASTEGNVTGMFAG |
| | | GGGGMAEMKTDAATLAQEAGNFERISGDLKTQIDQVESTAGSLQGQWRGAAGTAA |
| | | QAAVVRFQEAANKQKQELDEISTNIRQAGVQYSRADEEQQQALSSQMGFTSASNTK |
| | | VDKKVEPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEALHN |
| | | HYTQKSLSLSTGKSEKDEL |
| 43 | Hinge region | EPRGPTIKPSPPCKCP |
| | from a mouse | |
| | IgG2a (wild | |
| | type) | |
| 44 | Hinge region | EPKSCDKTHTCPPCP |
| | from a human | |
| | IgG1 (wild type) | |
| 48 | Cov2-PCF | MKNTSSLCLLLLVVLCSLTCNSGQATPQNITDLCAEYHNTQIHTLNDKIFSYTESLAG |
| | including the | KREMAIITFKNGATFQVEVPGSQHIDSQKKAIERMKDTLRIAYLTEAKVEKLCVWN |
| | IgM tail piece | NKTPHAIAAISMANAEAAAKEAAAKEAAAKAQPTESIVRFPNITNLCPFGEVFNATR |
| | (mu-tp) | FASVYAWNRKRISNCVADYSVLYNSASFSTFKCYGVSPTKLNDLCFTNVYADSFVIRG |
| | | DEVRQIAPGQTGKIADYNYKLPDDFTGCVIAWNSNNLDSKVGGNYNYLYRLFRKSN |
| | | LKPFERDISTEIYQAGSTPCNGVEGFNCYFPLQSYGFQPTNGVGYQPYRVVVLSFELL |
| | | HAPASNTKVDKKVEPKSSDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFSCSV |
| | | MHEALHNHYTQKSLSLSTGKPTLYNVSLVMSDTAGTCYSEKDEL |

### Vector Construct and Expression in Plants

To express the PCF fusion protein in tobacco, the plant expression vector, pTRAk.2 (Sack et al., 2007), which contains two cassettes for foreign gene expression and antibiotics resistance, was used. It contains the following basic components: Scaffold Attachment Region (SAR) of the tobacco Rb7 gene (GenBank U67919); Cauliflower mosaic virus (CaMV) 35S promoter with duplicated transcriptional enhancer; Tobacco Etch Virus (TEV) 5′UTR (GenBank M15239); Polyadenylation signal for CaMV 35S transcript.

The synthesized gene encoding antigen fused with the signal and ER retention peptide gene sequences, was subcloned into pTRAK.2 using NcoI and XbaI restriction enzyme sites, by molecular cloning technology. For expression in tobacco, the pTRAK.2 construct containing PCF fusion gene was transformed into Agrobacterium strain GV3101 containing pMP90RK helper plasmid, by electroporation and selected on YENB medium (7.5 g of Bacto-yeast Extract, 8 g of Nutrient broth, pH 7.5), containing 50 μg/mL each of carbenicillin and rifampicin.

For transient expression in plant cells, vacuum infiltration method with Agrobacterium was performed. Briefly, the covered pots with about 6 to 8 week-old Nicotiana benthamiana plants were submerged in the liquid suspension of Agrobacterium, which was diluted to OD₆₀₀=0.1-0.2 in infiltration media (10 mM MES, 10 mM MgCl₂) and subjected to decreased pressure followed by rapid re-pressurization (Bechtold et al. 1993; Bechtold and Pelletier 1998; Tague and Mantis 2006). Then, the plants were kept in a short-day light cycle (8 h light/16 h dark; light intensity 80-100 μmol m-2 s-1) at 21° C. during day and 16° C. during night until harvesting the leaves at 5 to 7 days after infiltration.

### Purification of the PCF Fusion Protein

To extract plant-derived PCF fusion proteins, 5 to 7 days post-infiltrated leaves were frozen at −70° C., then homogenized in a blender with 2˜3 volumes Tris-HCL buffer (pH 8.0) containing 0.05% of sodium cholate hydrate (Sigma). The crude extracts were filtered through 3-layers of Miracloth (Calbiochem) and centrifuged at 13000 rpm for 50 min in a ROTINA 48R centrifuge (Hettich Zentrifugen). The supernatant was sterilized through a 0.22 μm filter before applying to a protein A agarose affinity column (Sigma). After extensive washing with Tris extraction buffer, the bound protein was eluted in 0.1 M glycine-HCl, pH 2.7, and the fractions neutralized by addition of 1 M Tris base (pH unadjusted). The eluted protein-containing fractions were combined and concentrated by ultrafiltration (Amicon® Ultra-15 100K device) followed by dialysis against PBS. The protein content was determined by measuring optical density at 280 nm using NanoDrop™ 2000/2000c Spectrophotometers (ThermoScience).

### Protein Analysis Using Gel Electrophoresis and Immunoblot Analysis

To confirm the expression of antigen-PCF in plant extracts, samples were run on 4-12% Bis-Tris gels (Life Technologies) using NuPAGE® MOPS SDS Running Buffers (Life Technologies). Following electrophoresis, gels were stained with InstantBlue (Expedeon) or subjected to Western blot analysis. The blotted membrane was blocked for 30 min with 5% (w/v) non-fat dried milk in PBS and incubated overnight with peroxidase-conjugated anti-human IgG antiserum (1:2500 dilution; The Binding Site) for detection of the IgG Fc portion, or with mouse anti-dengue virus monoclonal antibody (1:2500 dilution; Bio-Rad AbD Serotec) followed by anti-mouse IgG (light chain specific) peroxidase-conjugated antiserum (1:2500, Jackson ImmunoResearch), or with rabbit anti-CT polyclonal antibody (1:2500 dilution; Sigma) followed by anti-rabbit IgG peroxidase-conjugated antiserum (1:2500, Sigma). The blots were washed with PBS/0.01% Tween-20 (PBST) and developed using the ECL Plus Western blotting detection system (GE Healthcare).

To visualise high molecular weight structures in conventional SDS-PAGE or native gels, the purified recombinant proteins were separated on 3-8% Tris-Acetate gels using NuPAGE® Tris-Acetate SDS Running Buffer or 3-8% NativePAGE™ Bis-Tris gels (Life Technologies), followed by InstantBlue staining.

### Binding of the PCF Fusion Polypeptide to GM1 Ganglioside or Complement C1q

To confirm the binding of antigen PCF to GM1 (3.0 μg/mL of monosialoganglioside GM1, Sigma) or Complement C1q protein (10 μg/mL of human C1q (Calbiochem) was coated onto ELISA plates in PBS buffer (pH 7.4) and incubated overnight at 4° C. After blocking in 5% non-fat dry milk protein solution in PBS, 2-fold serial dilutions of samples in duplicate were added and incubated at 37° C. for 2 h. The PBS buffer alone or commercial human IgG antibody (Sigma) were used as the negative controls in the experiments. For the CTB detection of binding to GM1, peroxidase-conjugated anti-rabbit IgG antiserum (Sigma) followed by anti-CTB polyserum (Sigma) were used, at 1/2500 dilution. For the polymeric Fc binding to Ciq protein, peroxidase-conjugated anti-human IgG antiserum (The Binding Site) was used at 1/25000. The peroxidase reaction was developed by adding 50-100 μL of TMB substrate solution (Bethyl Laboratories, Inc) to each well. The reaction was stopped by addition of 50 μL/well of 2 M H₂SO₄ and the absorbance was determined at 450 nm using a Sunrise plate reader (Tecan, UK).

### Cell Surface Binding

To test the capacity of PCF fusion protein to bind to Fc-receptor bearing cells, U937 or THP1 monocytes (ATCC) grown in RPMI medium supplemented with 10% Foetal Bovine Serum (FBS) were used. 1 million cells were suspended in 100 μL 3% BSA in PBS buffer and incubated on ice for 2 h with 5 or 20 μg/mL of PCF. Unbound protein was removed by washing 2 times in binding buffer and 7.5 μL of secondary antibody [anti-human IgG-FITC antiserum, Fab2 fragment only (Sigma)] added, followed by incubation for a further 1 h on ice. After washing as before, cells were resuspended in 500 μL of binding buffer and analysed for green fluorescence in a Becton-Dickinson flow cytometer. Secondary antibody alone was used for background staining. The data was analyzed with FlowJo v10 software program.

### IgG FcgRIII Binding

The avidity of the D-PCF for human FcgRIII (CD16a) was measured using a Biacore X100 instrument (GE Life Sciences, Little Chalfont, UK). Briefly, 12000 RU of an anti-His antibody was immobilized on both flow channels of a CM5 sensor chip using amine coupling chemistry (His capture kit, GE Life Sciences). For the sample, recombinant human CD16a containing a His tag (1960-FC, R&D systems) diluted to 2 μg/ml in HBS-EP+ running buffer was captured on flow channel 2, to a level of 750 RU. 500 nM, 166.7 nM, 55.6 nM and 18.5 nM of D-PCF or hIgG (as an intermediate control) were injected over both flow channels with a contact time of 80 s and a flow rate of 30 μl/min, and dissociation monitored for at least 500 s. Regeneration of the surface was achieved by a 30 s pulse of 10 mM glycine pH 1.5.

### Immunization of CD64 Transgenic Mice

For immunisation with D-PCF, 12-20 weeks old inbred male and female FcgRI/CD64 mice kept under defined environmental conditions, were used. In a pilot experiment, three mice per group were immunized subcutaneously (prime) with 30 μg of D-PCF in 100 μL without an external adjuvant, at the base of tail. Negative control mice were immunised with saline solution. Mice were immunised two times subsequently (boost) with 20 μg PCF in 30 μL PBS, via intranasal route, at weeks 4 and 6, and were bled after each immunisation to monitor the antibody titres. At week 8, mice were sacrificed for bleeding by cardiac puncture and collection of bronchoalveolar lavage (BAL), collected in 0.5 ml PBS. The spleens were collected for analysis of T cell responses.

### Humoral Response

For dengue antigen-specific IgG or IgA antibody responses induced by D-PCF, sera and BAL were tested by ELISA. ELISA plates were coated with cEDIII antigen (10 μg/ml) and probed with either 10-fold diluted mouse pooled sera (end point of the experiment and after each immunisation) or by 3-fold serial dilutions in individual mice. Antigen-specific IgG, IgG1, IgG2a, and IgA responses were detected by peroxidase-conjugated sheep secondary antibodies (The Binding Site) following the protocol for ELISA as described above. The data was analyzed by GraphPad Prism 6 software.

### Cellular Responses

To obtain splenocytes, spleens were extracted aseptically from immunized mice, pooled and homogenised in 10 mL of complete RPMI medium (Sigma) using a 5-ml syringe plunger. The tissue was squeezed through a 70 μm cell strainer (BD Falcon™). The released cells were spun and the pellet resuspended in RPMI medium. To eliminate red blood cells, the pelleted cells were incubated with ACK lysing buffer (Gibco) for 3 min at 37° C. and washed two times with 25 ml complete medium. Triplicate cultures were seeded into 96 well U-bottom plates at a density of 3×10⁵ cells/well, in 200 μL medium and stimulated with 10 μg/mL of dengue antigen or PBS as a control. After incubation of cells for 48 h at 37° C., 100 μL of supernatant was removed for Th1/Th2 and IL-17 cytokine ELISA assay. The experimental procedure described by the manufacturer (Mouse Th1/The ELISA Ready-SET-Go kit; affymetrix eBioscience, USA) was then followed.

### Measurement of PCF Particle Size by Zetasizer

To investigate the size of particles, 10 μg/mL of each sample (three vaccine candidates against Dengue, SARS CoV-2 and TB) were applied to Zeta-sizer (Malvern Panalytical) which performs size measurements using a process called Dynamic Light Scattering (DLS) and the data analyzed. (Dynamic Light Scattering measures Brownian motion and relates this to the size of the particles. It does this by illuminating the particles with a laser and analysing the intensity fluctuations in the scattered light.)

### Statistical Analysis

The ELISA for GM1, C1q binding and humoral antibody detection were performed in duplicates and the values are shown as the mean+/−standard deviation. To measure T-cell cytokine production, the assays were performed in triplicates and the values are shown as the mean+/−standard deviation. The software of GraphPad Prism 6 and FlowJo v10 were used for graphs and statistical analyses, based on One Way Anova and Tukey's post-hoc test. Significant differences were considered when p was less than 0.05.

Reactivity of CoV2-PCF with Covid Immune Sera by ELISA

Two previously immunized donors (using the Pfizer COVID vaccine) were tested for serological reactivity to either CoV2-PCF or RBD, or RBD-Fc. ELISA plates were coated with different forms of RBD at 5 micrograms/ml, blocked and then incubated with sera from donors in 3-fold serial dilutions, starting from 1:20. Bound antibodies were detected with goat anti-human IgG secondary antibodies conjugated to peroxidase, following addition of peroxidase substrates (SigmaFast OPD).

### SARS CoV-2-PCF Mice Immunization and Tissue Collection

6-8 week old female wild-type BALB/c mice were used for immunisations, with nine animals in each group. Five animals from each group were used for assessing mucosal responses, while the remaining animals were used to assess systemic immunity (this was necessary due to incompatibility of different protocols for harvesting tissues). Groups included mock immunisation (Phosphate-buffer saline, PBS), CoV2-PCF, with or without adjuvant (Quil-A), systemic (subcutaneous, s.c.) priming followed by intranasal (i.n.) or s.c. boosting, and antigen alone (RBD). The amount of RBD antigen given was normalised to 10 μg/animal for all animals receiving SARS-CoV2-PCF or RBD. QuilA adjuvant was administered at 1 μg/animal for subcutaneous and 0.1 μg/animal for intranasal routes. All animals received three immunisations via the subcutaneous or intranasal route at two-week intervals followed by a cull two-weeks after the final immunisation. Serum samples were obtained throughout the experiment and at the cull timepoint for serological analysis while only the final bleeds were used for pseudovirus neutralisation assays. Lung-lavage was obtained at the termination of the experiment by intratracheally flashing the lung lumen with 1 ml of sterile PBS, followed by removal of cell debris and 5-fold concentration using Centricon spin columns. Lung and spleens were harvested and homogenised to generate single cell suspensions for cellular immunogenicity assays.

### Virus Neutralisation Assay

Pseudovirus stocks were generated using HEK293T cells using the X-tremeGENE™ 360 Transfection Reagent (Roche) with the following plasmids: p8.91, pLuc (Luciferase) and pCAGGGS-SARS-CoV-2 Spike. Cell culture supernatants were harvested and filtered through a 0.45 μm filter and stored at −20° C. The titre of pseudovirus stocks was determined by titration with ACE2/TMPRSS2 transfected HEK293T cells. Serum (heat-treated at 56° C. for 30 minutes) and bronchoalveolar lavage samples were tested for pseudovirus neutralising capability by incubating serial dilutions of samples with pseudovirus for 1 hour at 37° C., followed by incubation with HEK293T ACE2 TMPRSS2 cells for 48 hours at 37° C. in a humidified 5% CO2 incubator. Pseudovirus titre was assessed by adding Bright-glo (Promega) reagent and reading on the GloMax (Promega) microplate reader.

### Results

The innovative vaccine approach described herein is specifically designed to induce a fast-acting robust mucosal immune response in the lungs or gut, which are the target organs for infections caused via either the respiratory or the oral route. While mucosal application is preferred, systemic (i.e. injection) application is also considered, either alone, or in combination with subsequent mucosal application (in a prime-boost manner). The protein-only based vaccine platform derives its own adjuvanticity (i.e. is self-adjuvanting) from fusing the antigen to the IgG antibody heavy chain Fc fragment and to the non-toxic subunit of CTB. This results in the antigen being delivered directly to the Fc-receptor bearing antigen-presenting cells (APC) in the context of a strong CTB-mediated mucosal immune response. CTB receptor GM1 is broadly distributed in a variety of cell types, including mucosal lining epithelial cells, which ensures optimal access to the immune system. The inventors have shown that polymeric Fc-fusion protein (PIGS, polymeric IgG scaffold) alone is highly immunogenic against dengue and the Porcine Epidemic Diarrhoea (PED) coronavirus, and that addition of the CTB component results in surprisingly superior, long-lasting IgA responses to a dengue antigen in broncho-alveolar lavage in mice.

### Example 1—Expression of PCF Polypeptide in Plants

The inventors had previously developed a Poly-IgG Fc fusion protein for efficient systemic delivery of a dengue antigen to antigen presenting cells (APC), that was shown to be highly immunogenic (Kim, 2017, 2018). To enhance mucosal immune responses, the inventors have now further modified this concept by the addition of CTB so that the resulting fusion construct (see FIGS. 1A and 1B) includes CTB, IgG-Fc and the dengue antigen, cEDIII, to generate dengue-PCF (D-PCF). The expression of PCF fusion protein consisting of all three components was verified by immunoblotting with each component specific antibodies (see FIG. 2). The polymeric forms (see FIGS. 1A(c) and 1B(c)) with higher molecular weights (Mw) than monomer of PCF (approximately 104 kDa) and its single chain (approximately 52 kDa), were detected under non-reducing and reducing conditions, respectively (see FIG. 2a). Then, Cov2-PCF was next constructed by replacing the dengue antigen with SARS-CoV-2 antigen, RBD, and simultaneously tested the effect of two different types of linkers (between CTB and the antigen), on expression and polymerization in Nicotiana benthamiana plants. The results showed no significant difference between the two linkers (FIG. 2b).

### Example 2—Functional Characterisation of D-PCF In Vitro and In Vivo

The bio-functionality of the new fusion protein was demonstrated by its binding to mucosal epithelial cells gangliosides through CTB (FIG. 3a), and human Fc-receptor bearing antigen presenting cells, APCs (FIG. 3b). The increased avidity (compared to monomeric IgG) of binding to the low affinity IgG receptor (Fc gamma RIIIa, CD16a) was demonstrated by surface-plasmon resonance measurements (FIG. 3c), suggesting that the PCF fusion protein can enhance antigen uptake and presentation by APC by targeting low affinity receptors, thus boosting cellular and humoral immune responses. This was confirmed by the detection of high titer antigen-specific antibodies and induction of cellular immune responses in immunized mice (see FIGS. 4 and 5). Furthermore, systemic prime and intranasal boost with D-PCF led to robust systemic and mucosal immune responses in the absence of any additional adjuvants. The titer of antigen-specific serum IgG was >103 after one systemic injection and >10⁵ after one or two nasal boosts (FIG. 4a). Both IgG1 and IgG2a antibodies were induced, indicating a balanced Th1/Th2 type immune response.

Importantly, D-PCF induced a strong mucosal IgA antibody response detected in bronco-alveolar lavage of immunized mice (FIG. 4b). The differences between individual mice might be explained by the different level of expression of CD64 (human IgG-Receptor I) in transgenic mice, needed for testing of the human version of D-PCF. As for IgG antibody responses, the cytokine profile in antigen-stimulated cultures of splenocytes from immunized mice, indicated a balanced Th1/Th2 cellular immune response, with evidence of a significant induction of IFN-8, IL-17 (Th1/Th17), IL-4 and IL-10 (Th2) (see FIG. 5).

### Example 3—Example of Antigens Used on the PCF Platform and Potential for Broad Application

Thus far, three versions of the PCF fusion polypeptide platform technology have been made, using Dengue, SARS CoV-2 and TB antigens (FIG. 6). To compare the expression pattern of the proteins, the SDS-PAGE-separated proteins were analyzed by immuno-detection with anti-CTB specific antibody, and the results showed that the expressed fusion proteins were denatured under the reducing conditions into the single chains of correct sizes of 51 kDa, 65 kDa and 61 kDa, for D-PCF, Cov2-PCF and TB-PCF, respectively (FIG. 6A). This takes into account the presence of N-glycosylation sites within the Fc fragment.

Immunoblotting analysis of the PCF fusion protein under non-reducing denaturing conditions (FIG. 6a, NR) showed the presence of a monomeric form (paired single chains) and higher polymers >250 KDa. Direct staining of purified proteins on a non-reducing, denaturing or native 3-8% gels, indicated the presence of monomers and polymers, with the latter ranging in size from 250-720 KDa (FIG. 6b). This result was consistent with the size-distribution of PCF forms measured by Zeta-sizer through dynamic light scattering (DLS) technology (FIG. 6c).

The particle size of obtained peaks and expected molecular weights are summarized in the table in FIG. 6c: D-PCF (24.36 and 122.4 nm for peak 1 and peak 2), SARS CoV-2 (32.67 and 220 nm for peak 1 and peak 2) and TB (50.57 and 255.0 nm in peak 1 and peak 2), with peak 1 corresponding to monomer and peak 2 to polymers. The PCF fusion protein embedding multiple Fc and CTB in polymeric structures, was capable of binding to C1q component of the Complement (via Fc) and GM1 (via CTB) ganglioside (FIG. 6d). TB-PCF showed less polymerization and binding to C1q and GM1 gangliosides compared to other two vaccine candidates, indicating antigen dependence during assembly, possibly due to structural constraints. Finally, all three versions of PCF fusion protein bound efficiently to the surface of U937 cells expressing Fc gamma receptors, by flow cytometry (FIG. 6e).

Currently, the presence of polymeric forms (as shown in FIG. 1c) is evidenced through SDS-PAGE and biological functionality (i.e. monomeric IgG does not bind to C1q, only multimeric forms or immune complexes do). However, the precise distribution of molecular forms and their relative proportions cannot be established by SDS-PAGE, as denaturing conditions lead to their dissociation. Further work on defining the size of polymers (that can range from dimer to pentamer, or greater), as well as their purification to a high-level homogeneity required for application, will be done by HPLC (4).

### Example 4—Optimization of a Protocol for Testing with Human Cells and Ex Vivo Tonsil Tissue

Referring to FIG. 7, there is shown that CoV2-PCF, when applied to growing cultures of human U937 cells at a range of concentrations of 5-50 μg/ml, did not exert any toxicity above the vehicle only (negative control, NC) over a period of 48 h. Diminished cell viability at 48 h is due to nutrient exhaustion in the culture, being equal for untreated and treated cells.

Referring to FIG. 8, there is shown that CoV2-PCF can bind to human tonsil culture subpopulations of immune cells. The binding could be detected with whole tonsil cell suspension, and when further analysed, it could be shown that majority of binding occurs through B cells and a to a low extent, through monocytes. Both B cells and monocytes are known to express Ig-Fc-gamma receptors and to be capable of immune complex binding, internalization and antigen presentation.

The inventors have demonstrated the feasibility of aerosolization of PCF using the classical Omron micro-nebulizer, suitable for human application. They demonstrated that aerosolization of CoV2-PCF resulted in a partial loss of the protein in the condensate, but this could be fully reversed by addition of 0.05% Tween-80 (suitable for human application), so that the protein is fully recovered in the aerosol condensate (FIG. 9A). They further demonstrated that the aerosolized PCF was capable of binding to GM1 (FIG. 9B) and Fc gamma receptors on U937 cells (FIG. 9C), in the same manner as before aerosolization, thus confirming full functionality.

### Example 5—Comparison of the Immunogenicity of the Polymeric Fusion Polypeptide to that of Monomeric Polypeptides

The inventors have demonstrated that antibodies from two different immunized donors (using the Pfizer vaccine) bound effectively to RBD moiety within the CoV2-PCF polypeptide, by ELISA. This shows that B cell epitopes on the RBD antigen within the construct are not blocked or masked, which is important for inducing antibody responses in vivo. Moreover, they have demonstrated superior binding of pre-existing antibodies in immunized hosts to polymeric CoV2-PCF compared to monomeric RBD or RBD-Fc (FIG. 10). This is likely due to the conformational characteristics of RBD within the polymeric polypeptide more closely resembling the natural structure of RBD within the viral spike protein, than the monomeric forms of recombinant RBD.

### Example 6—Assessment of the Systemic Immunogenicity of the SARS CoV-2-PCF Vaccine in Mice

The inventors have demonstrated potent immunogenicity of the SARS CoV—PCF vaccine in mice following different immunisations regimens. Thus, they showed that systemic priming of mice by injection, followed by boosting by either injection or intranasal administration, induced comparable IgG antibody responses in sera (FIG. 11). They also showed that vaccine formulation with an adjuvant (Quil-A) further enhanced systemic antibody responses.

### Example 7—Efficacy of the SARS CoV-2 Vaccine in Intranasal/Mucosal Administration

The inventors have also shown in mice that only vaccination regimens that included intranasal boosting with CoV—PCF induced mucosal antibody responses as represented by IgA in the broncho-alveolar lavage (BAL). Of the various immunisation groups and regimens, only animals that received two intranasal doses of CoV2-PCF, whether alone or with the Quil-A adjuvant, displayed high levels of IgA in BAL, whereas animals that were immunised only systemically (by subcutaneous injection) did not perform as well (FIG. 12).

Furthermore, they also showed that addition of adjuvant further enhanced vaccine-induced IgA response in BAL. Likewise, when assessing for presence of resident memory T cells (Trm) in the lung tissue, the mucosally boosted animals displayed higher frequency than the systemically only immunised animals (FIG. 13). The Trm cells are thought to be important for mounting a rapid immune response to viral infection. Collectively, these data support the conclusion that the mucosal route of administration is the most effective in inducing local cellular and humoral immunity in the lungs.

### Example 8—Virus Neutralising Potential of Vaccine-Induced Systemic and Mucosal Antibodies

The inventors have shown that SARS CoV—PCF vaccine induced antibodies in mice can neutralise the virus in a dose dependent manner in an in vitro cell infection model. They showed that intranasally boosted mice displayed superior virus neutralising potency in both sera and BAL, compared to only systemically immunised mice (FIG. 14). This is represented by 50% viral neutralisation achieved with lower sera (1:200) and BAL (1:100) than for systemic immunisation with the same vaccine. Furthermore, vaccine formulation with the Quil-A adjuvant induced still higher neutralising antibody responses in both sera (1:1000) and BAL (1:400). These data show that mucosally immunised mice with CoV2-PCF, display virus neutralising potential with both their sera and BAL, and that this potential is further increased by addition of an adjuvant.

### DISCUSSION AND CONCLUSIONS

As demonstrated in the Examples, the inventors have developed a highly innovative and surprisingly effective vaccine approach that is designed to induce a fast-acting robust mucosal immune response in the lungs and gut, the target organs for infection. The rationale in this innovative approach is to incorporate, into the same single polypeptide, (i) a non-toxic CTB, a highly efficient mucosal adjuvant currently licensed as a component of the mucosal Dukoral cholera vaccine, with (ii) a target antigen, as well as (iii) the IgG-Fc fragment, within the structure of the fusion protein vaccine. This way, both the intrinsic molecular adjuvant (CTB) and the antigen (Ag) are delivered within the same fusion protein. In addition, the same fusion protein comprises the Ig-Fc (preferably, the truncated CH1), which serves to mediate enhanced uptake of the fusion protein by the antigen presenting cells, resulting in a robust immune response.

The inventors have demonstrated the feasibility of construction and expression of the proposed PCF vaccine platform in plants, in the context of three quite different antigens, i.e. dengue, SARS-Cov2 and TB. They have demonstrated the presence of polymeric forms (i.e. pentameric structures) through bio-physical analysis and biological assays of functionality, including binding to C1q, GM1 and IgG-Fc receptors on cells. For D-PCF, the inventors demonstrated immunogenicity in mice following systemic priming and mucosal boost. Thus, strong antibody responses were detected in both sera and mucosal fluids, as well as cellular responses in the spleen. As described above, it is therefore, clear that the PCF vaccine platform has a significant potential to be developed as a method of choice for rapid vaccine development against certain infectious diseases, and in particular those that affect mucosal tissues.

Furthermore, the inventors have shown that B cell epitopes on the antigens within the constructs of the invention are not blocked or masked, which is essential for inducing antibody responses in vivo. They have also demonstrated superior binding of pre-existing antibodies in immunised hosts to polymeric constructs of the invention compared to monomeric constructs available in the art. The improved binding or targeting of APCs via polymeric Fc observed with the fusion polypeptide of the invention results from the intelligent engineering of the fusion polypeptide in which the synergistic activities of the different linkers present in the construct (such as CH1, GP, EK, and μ-tp) significantly increase the flexibility of the construct and subsequently improve its polymerisation.

Furthermore, as shown in the examples, the inventors have successfully demonstrated that the fusion polypeptides of the invention produce potent immunogenicity in vivo, regardless of the immunisation routes or regiments. Indeed, boosting mice by intravenous injection or intranasal administration induced comparable antibody responses in sera after systemic priming. Surprisingly, however, the inventors have also demonstrated that vaccination regimens that included intranasal boosting with a fusion polypeptide of the invention induced mucosal antibody responses, and mice cohorts which received two intranasal doses of a fusion polypeptide vaccine of the invention either alone or with an adjuvant displayed higher mucosal antibody responses compared to those that only received a systemic immunisation.

Similarly, a higher frequency of resident memory T cells in the lung tissue was observed with mucosally boosted animals compared to animals immunised via systemic injections only. The Examples also demonstrate that the addition of exogenous adjuvants further enhances the immunogenicity of the fusion polypeptides of the invention.

The inventors have shown that SARS CoV—PCF vaccine induced antibodies in mice can neutralise the virus in a dose dependent manner in an in vitro cell infection model. They showed that intranasally boosted mice displayed superior virus neutralising potency in both sera and BAL, compared to only systemically immunised mice.

These combined data strongly support the improved effectiveness of the fusion polypeptides of the invention in mucosal applications by aerosolisation without loss of material or functionality. In addition to inducing a mucosal immune response, aerosol formulations for mucosal applications present several advantages, including increased vaccine stability and shelf-life and possible application through disposable inhalers, which decreases the need for trained healthcare personnel and, therefore, facilitates mass-vaccination campaigns.

### REFERENCES

1. Van Riel, D. A. J & de Wit, E. Next-generation vaccine platforms for COVID-19. Nature Materials 19, 810-812 (2020). DOI https://doi.org/10.1038/s41563-020-0746-0
2. Kim, M. Y., et al., Plant-expressed Fc-fusion protein tetravalent dengue vaccine with inherent adjuvant properties. Plant Biotechnology Journal, 2018.
3. Kim, M. Y., et al., Novel vaccination approach for dengue infection based on recombinant immune complex universal platform. Vaccine, 2015.
4. Kim, M. Y., et al., Molecular engineering and plant expression of an immunoglobulin heavy chain scaffold for delivery of a dengue vaccine candidate. Plant Biotechnol J, 2017. 15 (12): p. 1590-1601.
5. Kim, M. Y., et al., Oral immunisation of mice with transgenic rice calli expressing cholera toxin B subunit fused to consensus dengue cEDIII antigen induces antibodies to all four dengue serotypes. Plant Mol Biol, 2016.
6. Huy, N. X. and M. Y. Kim, Overexpression and oral immunogenicity of a dengue antigen transiently expressed in Nicotiana benthamiana. Plant Cell Tissue and Organ Culture, 2017.
7. Kim, M. Y., et al., Expression of a cholera toxin B subunit and consensus dengue virus envelope protein domain III fusion gene in transgenic rice callus. Plant Cell Tissue and Organ Culture, 2013.
8. Kurokawa, S., et al., MucoRice-cholera Toxin B-subunit, a Rice-based Oral Cholera Vaccine, Down-regulates the Expression of α-Amylase/trypsin Inhibitor-like Protein Family as Major Rice Allergens. Journal of Proteome Research, 2013.
9. Lyon, G. M., et al., Clinical care of two patients with Ebola virus disease in the United States. N Engl J Med, 2014.
10. Pillet, S., et al., Immunogenicity and safety of a quadrivalent plant-derived virus like particle influenza vaccine candidate-Two randomized Phase II clinical trials in 18 to 49 and >50 years old adults. PLOS ONE, 2019.
11. Ward, B J et al Phase 1 randomized trial of a plant-derived virus-like particle vaccine for COVID-19, Nat. Med. 2021.
12. DeWolf M. J. S., et al., Tryptophan residues of cholera toxin and its A and B protomers. J. Biol. Chem, 1981.
13. Hilbush B. S., and Levine J. M., Modulation of a Ca2+ signaling pathway by GM1 ganglioside in PC12 cells, J Biol Chem, 1992.
14. Morrison W. J., et al., Ganglioside (GM1)-treated T cells shed CD4. Immunopharmacology, 1991.
15. Weis F. M., and Davis R. J., Regulation of epidermal growth factor receptor signal transduction. Role of gangliosides, J Biol Chem, 1990.

1. A fusion polypeptide comprising:
(i) a first amino acid sequence comprising a non-toxic B subunit of an AB₅ Toxin, or a fragment or variant thereof;
(ii) a second amino acid sequence comprising an immunoglobulin Fc region (Ig-Fc), or a fragment or variant thereof; and
(iii) a third amino acid sequence comprising an antigen, or a fragment or variant thereof.

2. The fusion polypeptide according to claim 1, wherein the first amino acid sequence comprising the AB₅ toxin B subunit, or a fragment or variant thereof, is disposed at or towards the N-terminus of the fusion polypeptide, and the second amino acid sequence comprising the Ig-Fc region, or a fragment or variant thereof is disposed at or towards the C-terminus of the polypeptide, and the third amino acid sequence comprising the antigen, or a fragment or variant thereof, is disposed in between the first and second amino acid sequences.

3. The fusion polypeptide according to either claim 1 or claim 2, wherein the AB₅ toxin B subunit is a haemolytic-uraemic B subunit, optionally wherein the haemolytic-uraemic B subunit is represented by Escherichia coli's heat labile enterotoxin (LT) subunit B.

4. The fusion polypeptide according to either claim 1 or claim 2, wherein the AB₅ toxin B subunit is a dysenteric B subunit, optionally wherein the dysenteric B subunit is represented by Shigella dysenteriae's shiga toxin (Stx) subunit B.

5. The fusion polypeptide according to either claim 1 or claim 2, wherein the AB₅ toxin B subunit is a cholera toxin B subunit (CTB), optionally wherein the cholera toxin B subunit is represented by Vibrio cholerae's cholera toxin subunit B.

6. The fusion polypeptide according to any one of claim 1, 2, or 5, wherein the first amino acid sequence comprises or consists of an amino acid sequence substantially as set out in SEQ ID No:1, or a fragment or variant thereof and/or is encoded by a nucleotide sequence substantially as set out in SEQ ID No:2, or a fragment or variant thereof.

7. The fusion polypeptide according to any preceding claim, wherein the Ig-Fc region, or a fragment or variant thereof is selected from IgA, IgD, IgE, IgG and/or IgM.

8. The fusion polypeptide according to any preceding claim, wherein the Ig-Fc region, or a fragment or variant thereof is an IgG, preferably (i) human IgG, more preferably a human IgG1, or (ii) mouse IgG, more preferably a mouse IgG2a.

9. The fusion polypeptide according to any preceding claim, wherein the Ig-Fc region, or a fragment or variant thereof comprises a CH domain or CH₃ domain of an immunoglobulin.

10. The fusion polypeptide according to any preceding claim, wherein the second amino acid sequence comprises or consists of an amino acid sequence substantially as set out in SEQ ID No:3, or a fragment or variant thereof and/or is encoded by a nucleotide sequence substantially as set out in SEQ ID No:4, or a fragment or variant thereof.

11. The fusion polypeptide according to any preceding claim, wherein the second amino acid sequence comprises or consists of an amino acid sequence substantially as set out in SEQ ID No:5, or a fragment or variant thereof and/or is encoded by a nucleotide sequence substantially as set out in SEQ ID No:6, or a fragment or variant thereof.

12. The fusion polypeptide according to any preceding claim, wherein the Ig-Fc region, or a fragment or variant thereof comprises a CH domain or CH₂ domain of an immunoglobulin.

13. The fusion polypeptide according to any preceding claim, wherein the second amino acid sequence comprises or consists of an amino acid sequence substantially as set out in SEQ ID No:7, or a fragment or variant thereof and/or is encoded by a nucleotide sequence substantially as set out in SEQ ID No:8, or a fragment or variant thereof.

14. The fusion polypeptide according to any preceding claim, wherein the second amino acid sequence comprises or consists of an amino acid sequence substantially as set out in SEQ ID No:9, or a fragment or variant thereof and/or is encoded by a nucleotide sequence substantially as set out in SEQ ID No: 10, or a fragment or variant thereof.

15. The fusion polypeptide according to any one of claims 12-14, wherein the CH₂ domain is disposed N-terminal of the CH₃ domain in the Ig-Fc region, or a fragment or variant thereof, and preferably wherein the Ig-Fc region, or a fragment or variant thereof comprises a CH₂—CH₃ domain of an immunoglobulin.

16. The fusion polypeptide according to any preceding claim, wherein the fusion polypeptide comprises a fourth amino acid sequence comprising a hinge region of an immunoglobulin, optionally wherein the hinge region is disposed N-terminal of the second amino acid comprising the Ig-Fc region, or a fragment or variant thereof.

17. The fusion polypeptide according to claim 16, wherein the hinge region is selected from the hinge region of IgA, IgD, IgG, and/or the C domain of IgE and IgM.

18. The fusion polypeptide according to either claim 16 or claim 17, wherein the fourth amino acid sequence comprises or consists of an amino acid sequence substantially as set out in SEQ ID No:11, or a fragment or variant thereof and/or is encoded by a nucleotide sequence substantially as set out in SEQ ID No: 12, or a fragment or variant thereof.

19. The fusion polypeptide according to any one of claims 16-18, wherein the hinge region comprises a point mutation in SEQ ID No:11 at position Ile234, which is most preferably Ile234Asn.

20. The fusion polypeptide according to either claim 16 or claim 17, wherein the fourth amino acid sequence comprises or consists of an amino acid sequence substantially as set out in SEQ ID No:13, or a fragment or variant thereof and/or is encoded by a nucleotide sequence substantially as set out in SEQ ID No:14, or a fragment or variant thereof.

21. The fusion polypeptide according to claim 20, wherein the hinge region comprises a point mutation in SEQ ID No:13 at position Cys230, which is most preferably Cys230Ser.

22. The fusion polypeptide according to any one of claims 16-21, wherein the hinge region is disposed N-terminal of the CH₂ and/or CH₃ domain in the Ig-Fc region, or a fragment or variant thereof, preferably wherein the fusion polypeptide comprises a hinge-CH₂—CH₃ domain of an immunoglobulin.

23. The fusion polypeptide according to any preceding claim, wherein the fusion polypeptide comprises a fifth amino acid sequence comprising a CH₁ domain of an immunoglobulin, or a truncation thereof, optionally wherein the CH₁ domain is the full-length CH₁ domain of IgA, IgD, IgE, IgG and/or IgM.

24. The fusion polypeptide according to claim 23, wherein the fusion polypeptide thereof comprises a truncated CH₁ domain of an immunoglobulin, wherein at least the last 5, 6, 7, 8, 9 or 10 amino acid residues from the C-terminus, which correspond to the final β-strand of the CH₁ domain of an immunoglobulin are absent, deleted or removed, optionally wherein the fusion polypeptide comprises a ΔCH₁-hinge-CH₂—CH₃ domain of an immunoglobulin.

25. The fusion polypeptide according to either claim 23 or claim 24, wherein the CH₁ domain of an immunoglobulin, or a truncation thereof is selected from IgA, IgD, IgE, IgG and/or IgM.

26. The fusion polypeptide according to any one of claims 23-25, wherein the fifth amino acid sequence comprises or consists of an amino acid sequence substantially as set out in SEQ ID No:15, or a fragment or variant thereof and/or is encoded by a nucleotide sequence substantially as set out in SEQ ID No: 16, or a fragment or variant thereof.

27. The fusion polypeptide according to any one of claims 23-25, wherein the fifth amino acid sequence comprises or consists of an amino acid sequence substantially as set out in SEQ ID No:17, or a fragment or variant thereof and/or is encoded by a nucleotide sequence substantially as set out in SEQ ID No:18, or a fragment or variant thereof.

28. The fusion polypeptide according to any preceding claim, wherein the fusion polypeptide of the invention comprises a sixth amino acid sequence comprising a tailpiece domain of an immunoglobulin, or a fragment thereof, preferably wherein the immunoglobulin tailpiece domain or fragment thereof is the tailpiece domain of IgA, IgD, IgE, IgG and/or IgM, more preferably IgM.

29. The fusion polypeptide according to claim 28, wherein the sixth amino acid sequence comprises or consists of an amino acid sequence substantially as set out in SEQ ID No:45, or a fragment or variant thereof and/or is encoded by a nucleotide sequence substantially as set out in SEQ ID No:46, or a fragment or variant thereof.

30. The fusion polypeptide according to any preceding claim, wherein the third amino acid sequence comprising the antigen, or a fragment or variant thereof, is disposed between the first and second amino acid sequences, and most preferably it is disposed N-terminal of the fourth and fifth amino acid sequences.

31. The fusion polypeptide according to any preceding claim, wherein the antigen, or a fragment or variant thereof is derived or isolated from a bacterium, virus, fungus or protozoan.

32. The fusion polypeptide according to any preceding claim, wherein the antigen, or fragment or variant thereof, is a tumour-associated antigen.

33. The fusion polypeptide according to any preceding claim, wherein the third amino acid sequence comprises or consists of an amino acid sequence substantially as set out in SEQ ID No:19, or a fragment or variant thereof and/or is encoded by a nucleotide sequence substantially as set out in SEQ ID No:20, or a fragment or variant thereof.

34. The fusion polypeptide according to any preceding claim, wherein the third amino acid sequence comprises or consists of an amino acid sequence substantially as set out in SEQ ID No:20, or a fragment or variant thereof and/or is encoded by a nucleotide sequence substantially as set out in SEQ ID No: 22, or a fragment or variant thereof.

35. The fusion polypeptide according to any preceding claim, wherein the third amino acid sequence comprises or consists of an amino acid sequence substantially as set out in SEQ ID No: 23, or a fragment or variant thereof and/or is encoded by a nucleotide sequence substantially as set out in SEQ ID No: 24, or a fragment or variant thereof.

36. The fusion polypeptide according to any preceding claim, wherein the fusion polypeptide is a single polypeptide monomer or polymerises, whereby a plurality of polypeptide monomers aggregate, combine or fuse together.

37. The fusion polypeptide according to any preceding claim, wherein the fusion polypeptide comprises a signal peptide, which improves the level of expression and/or polymerisation of the fusion polypeptide in a host cell, preferably wherein the signal peptide is disposed at or towards the N-terminus of the fusion polypeptide.

38. The fusion polypeptide according to claim 35, wherein the signal peptide comprises an ER to Golgi trafficking signal peptide, optionally wherein the signal peptide comprises or consists of an amino acid sequence substantially as set out in SEQ ID No: 25, or a fragment or variant thereof and/or is encoded by a nucleotide sequence substantially as set out in SEQ ID No: 26, or a fragment or variant thereof.

39. The fusion polypeptide according to claim 35, wherein the signal peptide comprises or consists of an amino acid sequence substantially as set out in SEQ ID No: 27, or a fragment or variant thereof and/or is encoded by a nucleotide sequence substantially as set out in SEQ ID No: 28, or a fragment or variant thereof.

40. The fusion polypeptide according to any preceding claim, wherein the fusion polypeptide comprises a retrieval signal peptide, which improves the level of expression and/or polymerisation of the fusion polypeptide in a host cell, preferably wherein the retrieval signal peptide is disposed at or towards the C-terminus of the fusion polypeptide.

41. The fusion polypeptide according to claim 38, wherein the retrieval signal peptide comprises an ER retention signal peptide, optionally wherein the ER retention signal peptide comprises or consists of an amino acid sequence substantially as set out in SEQ ID No: 29, or a fragment or variant thereof and/or is encoded by a nucleotide sequence substantially as set out in SEQ ID No: 30, or a fragment or variant thereof.

42. The fusion polypeptide according to any preceding claim, wherein the fusion polypeptide comprises one or more linker peptide, which is disposed between first amino acid sequence, the second amino acid sequence and/or the third amino acid sequence, preferably wherein the polypeptide comprises a linker peptide disposed between the first amino acid sequence comprising the AB₅ toxin B subunit, or a fragment or a variant thereof, and the third amino acid sequence comprising the antigen, or a fragment of variant thereof.

43. The fusion polypeptide according to claim 40, wherein the linker peptide comprises or consists of an amino acid sequence substantially as set out in SEQ ID No: 31, or a fragment or variant thereof and/or is encoded by a nucleotide sequence substantially as set out in SEQ ID No: 32, or a fragment or variant thereof.

44. The fusion polypeptide according to claim 40, wherein the linker peptide comprises or consists of an amino acid sequence substantially as set out in SEQ ID No: 33, or a fragment or variant thereof and/or is encoded by a nucleotide sequence substantially as set out in SEQ ID No: 34, or a fragment or variant thereof.

45. The fusion polypeptide according to any preceding claim, wherein the fusion polypeptide comprises or consists of an amino acid sequence substantially as set out in SEQ ID No: 35, 36, 37, 38, 47, 48, 49 or 50, or a fragment or variant thereof.

46. A nucleic acid encoding the fusion polypeptide according to any one of claims 1-45.

47. The nucleic acid according to claim 46, wherein the nucleic acid comprises or consists of a nucleotide sequence substantially as set out in SEQ ID No: 39, 40, 41, 42, 51, 52, 53 or 54, or a fragment or variant thereof.

48. An expression cassette comprising the nucleic acid according to either claim 46 or 47.

49. A recombinant vector comprising the nucleic acid according to either claim 46 or 47 or the expression cassette according to claim 48.

50. A host cell comprising the recombinant vector according to claim 49, optionally wherein the host cell is a bacterial, yeast, viral, fungal, plant, mammalian or insect cell.

51. A method for producing the fusion polypeptide according to any one of claims 1-45, the method comprising the steps of:
(a) (i) introducing, into a host cell, the recombinant vector according to claim 49; and
(ii) culturing the host cell under conditions to result in the production of the fusion polypeptide according to any one of claims 1-45; or
(b) translating the polypeptide from the vector according to claim 49.

52. The method according to claim 51, wherein the host cell is a plant cell.

53. A fusion polypeptide obtained, or obtainable, by the method according to either claim 51 or claim 52.

54. A pharmaceutical composition comprising the fusion polypeptide according to any one of claims 1-45 or claim 53, the nucleic acid sequence according to either claim 46 or 47, the expression cassette according to claim 48 or the recombinant vector according to claim 49, and a pharmaceutically acceptable vehicle.

55. A process for making the pharmaceutical composition according to claim 54, the method comprising contacting the fusion polypeptide according to any one of claims 1-45 or claim 53, the nucleic acid sequence according to either claim 46 or 47, the expression cassette according to claim 48 or the recombinant vector according to claim 49 with a pharmaceutically acceptable vehicle.

56. The fusion polypeptide according to any one of claims 1-45 or claim 53, the nucleic acid sequence according to either claim 46 or 47, the expression cassette according to claim 48 or the recombinant vector according to claim 49 or the pharmaceutical composition according to claim 54, for use as a medicament, or in therapy or prophylaxis.

57. A vaccine comprising the fusion polypeptide according to any one of claims 1-45 or claim 53, the nucleic acid sequence according to either claim 46 or 47, the expression cassette according to claim 48 or the recombinant vector according to claim 49 or the pharmaceutical composition according to claim 54.

58. The fusion polypeptide according to any one of claims 1-45 or claim 53, the nucleic acid sequence according to either claim 46 or 47, the expression cassette according to claim 48 or the recombinant vector according to claim 49, the pharmaceutical composition according to claim 54, or the vaccine according to claim 57, for use in treating, preventing or ameliorating an infection or cancer, optionally wherein the infection is caused by a micro-organism, such as a bacterium, virus, fungus or protozoan.

59. The fusion polypeptide according to any one of claims 1-45 or claim 53, the nucleic acid sequence according to either claim 46 or 47, the expression cassette according to claim 46 or the recombinant vector according to claim 49, the pharmaceutical composition according to claim 54, or the vaccine according to claim 57, for use in stimulating an immune response in a subject, optionally wherein the polypeptide, nucleic acid, cassette, vector, composition or vaccine is mucosally administered, preferably intranasally administered.
